SEQUENCE LISTING (114 sequences)

SEQ ID NO: 1 - protein, 115 aa, synthetic construct
EVKLVESGGGLVQPGGSLRLSCATSGFTFTDYYMSWVRQPPGKALEWLGFIRNKANGYRTEYSASVKGRFTISRDNSQSILYLQMNTLRAEDSATYYCASLSAYWGQGTLVTVSA

SEQ ID NO: 2 - DNA, 345 bp, synthetic construct
  source 1..345: mol_type = other DNA
gaggtgaagctggtggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacagaacagagtacagtgcatctgtgaagggtcggttcaccatctccagagataattcccaaagcatcctctatcttcaaatgaacaccctgagagctgaggacagtgccacttattactgtgcaagcttatctgcttactggggccaagggactctggtcactgtctctgca

SEQ ID NO: 3 - protein, 8 aa, synthetic construct
GFTFTDYY

SEQ ID NO: 4 - protein, 10 aa, synthetic construct
IRNKANGYRT

SEQ ID NO: 5 - protein, 6 aa, synthetic construct
ASLSAY

SEQ ID NO: 6 - protein, 111 aa, synthetic construct
DIVMTQAAFSNPVTLGTSASISCRSSKSLLHSNGITYLYWFLQKPGQSPQLLIYQMSNLASGVPDRFSSSGSGTDFTLRISRVEADDVGVYYCAQNLELWTFGGGTKLEIK

SEQ ID NO: 7 - DNA, 333 bp, synthetic construct
  source 1..333: mol_type = other DNA
gatattgtgatgacgcaggctgcattctccaatccagtcactcttggaacatcagcttccatctcctgcaggtctagtaagagtctcctacatagtaatggcatcacttatttgtattggtttctgcagaagccaggccagtctcctcagctcctgatttatcagatgtccaaccttgcctcaggagtcccagacaggttcagtagcagtgggtcaggaactgatttcacactgagaatcagcagagtggaggctgacgatgtgggtgtttattactgtgctcaaaatctagaactgtggacgttcggtggaggcaccaagctggaaatcaaa

SEQ ID NO: 8 - protein, 11 aa, synthetic construct
KSLLHSNGITY

SEQ ID NO: 9 - skipped

SEQ ID NO: 10 - protein, 8 aa, synthetic construct
AQNLELWT

SEQ ID NO: 11 - protein, 113 aa, synthetic construct
EVQLQESGPSLVKPSQTLSLTCSVTGASITSGHWNWIRKFPGNKLEYMGYIRDSGTTYYNPSLKGRLSITRDTSKNQYYLQLSSVTAEDTATYYCVTTPFVYWGQGTLVTVSA

SEQ ID NO: 12 - DNA, 339 bp, synthetic construct
  source 1..339: mol_type = other DNA
gaggtgcagcttcaggagtcaggacctagcctcgtgaaaccttctcagactctgtccctcacctgttctgtcactggcgcctccatcaccagtggtcactggaactggatccggaaattccctggaaataaacttgaatatatggggtacataagggacagtggtaccacttactacaacccatctctcaaaggtcgactctccatcactcgagacacatccaagaaccagtactacctgcagttgagttctgtgactgctgaggacacagccacatattattgtgtgactacgccgtttgtttactggggccaagggactctggtcactgtctctgca

SEQ ID NO: 13 - protein, 8 aa, synthetic construct
GASITSGH

SEQ ID NO: 14 - protein, 7 aa, synthetic construct
IRDSGTT

SEQ ID NO: 15 - protein, 7 aa, synthetic construct
VTTPFVY

SEQ ID NO: 16 - protein, 107 aa, synthetic construct
DIKMTQSPSSMFASLGERVTITCKASQDINSYLSWFQQKPGKSPKTLFYRANRLIDGVPSRFSGSGSGQDYSLTISSLENEDMGIYYCLQYDEFPLTFGAGTKLELK

SEQ ID NO: 17 - DNA, 321 bp, synthetic construct
  source 1..321: mol_type = other DNA
gacatcaagatgacccagtctccatcttccatgtttgcatctctaggagagagagtcactatcacttgcaaggcgagtcaggacattaatagttatttaagttggttccagcagaaaccagggaaatctcctaagaccctattctatcgtgcaaatagattaatagatggggtcccatcaaggttcagtggcagtggatctgggcaagattattctctcaccatcagcagcctggagaatgaagatatgggaatttattactgtctacagtatgatgagtttcccctcacgttcggtgctgggaccaagctggagctgaaa

SEQ ID NO: 18 - protein, 6 aa, synthetic construct
QDINSY

SEQ ID NO: 19 - skipped

SEQ ID NO: 20 - protein, 9 aa, synthetic construct
LQYDEFPLT

SEQ ID NO: 21 - protein, 112 aa, synthetic construct
QVHLKESGPGLVAPSQSLSITCTVSGFSLFNYGVHWVRQPPGKGLEWLGVIWADGTINYNSALMSRLTITKDNIKSQVFLKMDSLKTDDTAIYYCARHFDYWGQGTTLTVSS

SEQ ID NO: 22 - DNA, 336 bp, synthetic construct
  source 1..336: mol_type = other DNA
caggtgcacctgaaggagtcaggacctggcctggtggcgccctcacagagcctgtccatcacttgcactgtctctggcttttcattattcaactatggtgtacactgggttcgccagcctccaggaaagggtctggagtggctgggagttatttgggctgacggaaccataaattataattcggctctcatgtccagactgaccatcaccaaagacaacatcaagagccaggttttcttaaaaatggacagtctgaaaactgacgacacagccatatactactgtgccagacactttgactactggggccaaggcaccactctcacagtctcctca

SEQ ID NO: 23 - protein, 8 aa, synthetic construct
GFSLFNYG

SEQ ID NO: 24 - protein, 7 aa, synthetic construct
IWADGTI

SEQ ID NO: 25 - protein, 6 aa, synthetic construct
ARHFDY

SEQ ID NO: 26 - protein, 112 aa, synthetic construct
DVLLTQIPLSLPVSLGDQASISCRASQNIVHSNGNTYLDWYLQKPGQSPKLLIYKVSNRFSGVPDRFSGSGSGTDFTLKISRVEAEDLGVYYCFQGSRLPLTFGVGTKLELK

SEQ ID NO: 27 - DNA, 336 bp, synthetic construct
  source 1..336: mol_type = other DNA
gatgttttgctgacccaaattccgctctccctgcctgtcagtcttggagatcaagcctccatctcctgcagagctagtcagaacattgtacatagtaatggaaacacttatttagattggtacctgcagaaaccaggccagtctccaaagctcctgatctacaaagtttccaatcgattttctggggtcccagacaggttcagtggcagtggatcagggacagatttcacactcaagatcagcagagtggaggctgaggatctgggagtttattattgttttcaaggttcacgtcttccgctcacgttcggtgttgggaccaagctggagctgaaa

SEQ ID NO: 28 - protein, 11 aa, synthetic construct
QNIVHSNGNTY

SEQ ID NO: 29 - skipped

SEQ ID NO: 30 - protein, 9 aa, synthetic construct
FQGSRLPLT

SEQ ID NO: 31 - protein, 119 aa, synthetic construct
DVKLVESGGGLVKLGGSLKLSCAASGFIFSSYYMSWVRQTPEKKLELVAAININGGSTYYPDTVKGRFTISRDNAKNTLYLQMSSLKSEDTALYYCGRHDHGYGWFAYWGQGTLVTVSA

SEQ ID NO: 32 - DNA, 357 bp, synthetic construct
  source 1..357: mol_type = other DNA
gacgtgaagctcgtggagtcggggggaggcttagtgaaacttggagggtccctgaaactctcctgtgcagcctctggattcattttcagtagctattacatgtcttgggttcgccagactccggagaagaagctggagttggtcgcagccattaatattaatggtggtagcacctactatccagacactgtgaagggccgattcaccatctccagagacaatgccaagaacaccctgtacctgcaaatgagcagtctgaagtctgaggacacagccttatattactgtggaagacatgaccatgggtacggctggtttgcttactggggccaagggactctggtcactgtctctgca

SEQ ID NO: 33 - protein, 8 aa, synthetic construct
GFIFSSYY

SEQ ID NO: 34 - protein, 8 aa, synthetic construct
ININGGST

SEQ ID NO: 35 - protein, 12 aa, synthetic construct
GRHDHGYGWFAY

SEQ ID NO: 36 - protein, 107 aa, synthetic construct
DIVMTQSQKVMSTSVGDRVSVTCKASQNVGRNIAWYQQKPGQSPKTLIYSASSRYSGVPDRFTGSGFGTYFILTINNVQSEDLAAYFCQQYNSYPWTFGGGTKLEIK

SEQ ID NO: 37 - DNA, 321 bp, synthetic construct
  source 1..321: mol_type = other DNA
gacattgtgatgacccagtctcaaaaagtcatgtccacatcagtaggagacagggtcagcgtcacctgcaaggccagtcagaatgtgggtaggaatatagcctggtatcaacagaaaccagggcaatctcctaaaacactgatatattcggcatcctcccggtacagtggagtccctgatcgcttcacaggcagtggatttgggacatatttcattctcaccatcaacaacgtgcagtctgaagacttggcagcatatttctgtcagcaatataatagttatccgtggacgttcggtggaggcaccaagctggaaatcaaa

SEQ ID NO: 38 - protein, 6 aa, synthetic construct
QNVGRN

SEQ ID NO: 39 - skipped

SEQ ID NO: 40 - protein, 9 aa, synthetic construct
QQYNSYPWT

SEQ ID NO: 41 - protein, 117 aa, synthetic construct
QIQLVQSGPELKNPGETVRISCKASGYTFTTAGIQWVQKMPGKGLKWIGWINTQSGVSKCAEDFKGRFALSMETYASTAYLQMSNLKKEDTATYFCARSNSGGFPYWGQGTLVTVSA

SEQ ID NO: 42 - DNA, 351 bp, synthetic construct
  source 1..351: mol_type = other DNA
caaatccagttggtgcagtcaggacctgagctgaagaaccctggagagacagtcaggatctcctgcaaggcttctgggtataccttcacaactgctggaatccagtgggtgcaaaagatgccaggaaagggtttgaagtggattggctggataaacacccagtctggagtgtcaaaatgtgcagaagacttcaagggtcgatttgccttgtccatggaaacctacgccagcactgcatatttacagatgagcaacctcaaaaaagaggacacggctacgtatttctgtgcgagatcgaactccggcggttttccttactggggccaagggactctggtcactgtctctgca

SEQ ID NO: 43 - protein, 8 aa, synthetic construct
GYTFTTAG

SEQ ID NO: 44 - protein, 8 aa, synthetic construct
INTQSGVS

SEQ ID NO: 45 - protein, 10 aa, synthetic construct
ARSNSGGFPY

SEQ ID NO: 46 - protein, 113 aa, synthetic construct
DIVMTQSPSSLAMSIGQKVTMSCKSSQSLFNSTTHKIYLAWYQQKPGQSPKLLVYFASTRQSGVPDRFIGSGSWTDFTLTINSVQAEDLADYFCQQHYTAPWTFGGGTKLEIR

SEQ ID NO: 47 - DNA, 339 bp, synthetic construct
  source 1..339: mol_type = other DNA
gacattgtgatgacacagtctccatcctccctggctatgtcaattggacagaaggtcactatgagctgcaagtccagtcagagcctttttaatagtaccactcacaagatctatttggcctggtaccagcagaaaccaggacagtctcctaaacttctggtatactttgcatccactagacaatctggggtccctgatcgtttcatcggcagtggatcttggacagatttcactcttaccatcaacagtgtgcaggctgaagacctggcagattacttctgtcagcaacattataccgctccgtggacgttcggtggaggcaccaagctggaaatcaga

SEQ ID NO: 48 - protein, 12 aa, synthetic construct
QSLFNSTTHKIY

SEQ ID NO: 49 - skipped

SEQ ID NO: 50 - protein, 9 aa, synthetic construct
QQHYTAPWT

SEQ ID NO: 51 - protein, 118 aa, synthetic construct
QIQLVQSGPELKKPGETVRISCKASGYTFTTVGMQWVQKMPGKGLRWIGWINTHSGVSKYAEDFKGRFAFSLETSASTAYLEISNLKNEDTATYFCARGNSNYGFPYWGQGTLVTVSA

SEQ ID NO: 52 - DNA, 354 bp, synthetic construct
  source 1..354: mol_type = other DNA
cagatccagttggtgcagtctggacctgagctgaagaagcctggagagacagtcaggatctcctgcaaggcttctgggtataccttcacaactgttggaatgcagtgggtgcaaaagatgccaggaaagggtttgaggtggattggctggataaacacccactctggagtgtcaaaatatgcagaagacttcaagggacggtttgccttctctttggaaacctctgccagcactgcatatttagagataagcaacctcaaaaatgaggacacggctacgtatttctgtgcgaggggaaatagtaactacgggtttccttactggggccaagggactctggtcactgtctctgca

SEQ ID NO: 53 - protein, 8 aa, synthetic construct
GYTFTTVG

SEQ ID NO: 54 - protein, 8 aa, synthetic construct
INTHSGVS

SEQ ID NO: 55 - protein, 11 aa, synthetic construct
ARGNSNYGFPY

SEQ ID NO: 56 - protein, 107 aa, synthetic construct
DIQMTQSSSSFSVSLGDRVTITCKASEDIYNRLAWYQQRPGNAPRLLISGATTLETGVPSRFSGSGSGKDYTLSITSLQTEDVATYYCQQYWSTPWTFGGGTKLEIK

SEQ ID NO: 57 - DNA, 321 bp, synthetic construct
  source 1..321: mol_type = other DNA
gacatccagatgacacaatcttcatcctccttttctgtatctctaggagacagagtcaccattacttgcaaggcaagtgaagacatatataatcggttagcctggtatcagcagagaccaggaaatgctcctaggctcttaatatctggtgcaaccactttggaaactggggttccttcaagattcagtggcagtggatctggaaaggattacactctcagcattaccagtcttcagactgaagatgttgctacttattactgtcaacaatattggagtactccgtggacgttcggtggaggcaccaagctggaaatcaaa

SEQ ID NO: 58 - protein, 6 aa, synthetic construct
EDIYNR

SEQ ID NO: 59 - skipped

SEQ ID NO: 60 - protein, 9 aa, synthetic construct
QQYWSTPWT

SEQ ID NO: 61 - protein, 116 aa, synthetic construct
DVQLQESGPGLVKPSQSLSLTCTVTGYSITSDYAWTWIRQFPGNKLEWMGYISYRSITGYNPSLKSRISINRDTSKNQFFLQLNSVTTEDTATYYCARFGYDGNYWGQGTTLTVSS

SEQ ID NO: 62 - DNA, 348 bp, synthetic construct
  source 1..348: mol_type = other DNA
gatgtgcagcttcaggagtcgggacctggcctggtgaaaccttctcagtctctgtccctcacctgcactgtcactggctactcaatcaccagtgattatgcctggacctggatccggcagtttcccggaaacaaactggagtggatgggctacataagctacagaagtatcactggctacaacccatctctcaaaagtcgaatttctatcaatcgagacacatccaagaaccagttcttcctgcagttgaattctgtgactactgaggacacagccacatattactgtgcaagatttggttatgacgggaactactggggccaaggcaccactctcacagtctcctca

SEQ ID NO: 63 - protein, 9 aa, synthetic construct
GYSITSDYA

SEQ ID NO: 64 - protein, 7 aa, synthetic construct
ISYRSIT

SEQ ID NO: 65 - protein, 9 aa, synthetic construct
ARFGYDGNY

SEQ ID NO: 66 - protein, 112 aa, synthetic construct
DVLMTQTPLSLPVSLGDQASISCRSSQNIVHSNGDTYLEWYLQKPGQSPKLLIYRVSNRFSGVPDRFSGSGSGTDFTLKISRVEAEDLGVYYCFQGSHVPLTFGAGTKLELK

SEQ ID NO: 67 - DNA, 336 bp, synthetic construct
  source 1..336: mol_type = other DNA
gatgttttgatgacccaaactccactctccctgcctgtcagtcttggagatcaagcctccatctcttgcagatctagtcagaacattgtacatagtaatggagacacctatttagaatggtacctgcagaaaccaggccagtctccaaagctcctgatctacagagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaa

SEQ ID NO: 68 - protein, 11 aa, synthetic construct
QNIVHSNGDTY

SEQ ID NO: 69 - skipped

SEQ ID NO: 70 - protein, 9 aa, synthetic construct
FQGSHVPLT

SEQ ID NO: 71 - protein, 113 aa, synthetic construct
EVLLQQSGPELVKPGTSVKIPCKASGYTFTDYNMDWVKQSHGKSLEWIGDINPDNGATVYNQKFKGKATLTVDKSSSTAYMELRSLTSEDTAVYYCVRGFDYWGQGTSVTVSS

SEQ ID NO: 72 - DNA, 339 bp, synthetic construct
  source 1..339: mol_type = other DNA
gaggtcctgctgcaacagtctggacctgagctggtgaagcctgggacttcagtgaagataccctgcaaggcttctggatacacattcactgactacaacatggactgggtgaagcagagccatggaaagagccttgagtggattggagatattaatcctgacaatggtgctactgtctacaaccagaagttcaagggcaaggccacattgactgttgacaagtcctccagcacagcctacatggagctccgcagcctgacatctgaggacactgcagtctattactgtgtaagagggttcgactactggggtcaaggaacctcagtcaccgtctcctca

SEQ ID NO: 73 - protein, 8 aa, synthetic construct
GYTFTDYN

SEQ ID NO: 74 - protein, 8 aa, synthetic construct
INPDNGAT

SEQ ID NO: 75 - protein, 6 aa, synthetic construct
VRGFDY

SEQ ID NO: 76 - protein, 112 aa, synthetic construct
DVLMTQTPLSLPVSLGDQASISCRSSQAIVHSNGNTYLEWFLQKPGQSPKLLVYKVSNRFSGVPDRFSGSGSGTDFTLRISRVEAEDLGVYYCFQSSHIPWTFGGGTKLEIK

SEQ ID NO: 77 - DNA, 336 bp, synthetic construct
  source 1..336: mol_type = other DNA
gatgttttgatgacccagactccactctccctgcctgtcagtcttggagatcaagcctccatctcgtgcagatctagtcaggccattgtacatagtaatggaaacacctatttagaatggttcctgcagaaaccaggccagtctccaaagctcctggtctacaaagtttccaaccgattttctggggtcccagacaggttcagtggcagtggatcagggacagatttcacactcaggatcagcagagtggaggctgaggatctgggagtttattactgctttcagagttctcatattccgtggacgttcggtggaggcaccaagctggaaatcaaa

SEQ ID NO: 78 - protein, 11 aa, synthetic construct
QAIVHSNGNTY

SEQ ID NO: 79 - skipped

SEQ ID NO: 80 - protein, 9 aa, synthetic construct
FQSSHIPWT

SEQ ID NO: 81 - protein, 240 aa, synthetic construct
MDMRVPAQLLGLLLLWLRGARCDIVMTQAAFSNPVTLGTSASISCRSSKSLLHSNGITYLYWFLQKPGQSPQLLIYQMSNLASGVPDRFSSSGSGTDFTLRISRVEADDVGVYYCAQNLELWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 82 - DNA, 723 bp, synthetic construct
  source 1..723: mol_type = other DNA
atggacatgagagtgcccgcccagctgctgggactgctgctgctgtggctgagaggagcgcgctgcgatattgtgatgacgcaggctgcattctccaatccagtcactcttggaacatcagcttccatctcctgcaggtctagtaagagtctcctacatagtaatggcatcacttatttgtattggtttctgcagaagccaggccagtctcctcagctcctgatttatcagatgtccaaccttgcctcaggagtcccagacaggttcagtagcagtgggtcaggaactgatttcacactgagaatcagcagagtggaggctgacgatgtgggtgtttattactgtgctcaaaatctagaactgtggacgttcggtggaggcaccaagctggaaatcaaacgtacggtggccgccccctccgtgtttatcttccctcccagcgacgagcagctgaagagcggcacagccagcgtggtgtgtctgctgaacaacttctaccctagagaggccaaggtgcagtggaaagtggacaacgctctgcagtccggcaacagccaagagagcgtgaccgagcaagacagcaaggacagcacctactctctgagcagcacactgacactgtccaaggccgactacgagaagcacaaggtgtacgcttgcgaggtgacccaccaaggactgagcagccccgtgaccaagagcttcaataggggcgagtgctga

SEQ ID NO: 83 - protein, 236 aa, synthetic construct
MDMRVPAQLLGLLLLWLRGARCDIKMTQSPSSMFASLGERVTITCKASQDINSYLSWFQQKPGKSPKTLFYRANRLIDGVPSRFSGSGSGQDYSLTISSLENEDMGIYYCLQYDEFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 84 - DNA, 711 bp, synthetic construct
  source 1..711: mol_type = other DNA
atggacatgagagtgcccgcccagctgctgggactgctgctgctgtggctgagaggagcgcgctgcgacatcaagatgacccagtctccatcttccatgtttgcatctctaggagagagagtcactatcacttgcaaggcgagtcaggacattaatagttatttaagttggttccagcagaaaccagggaaatctcctaagaccctattctatcgtgcaaatagattaatagatggggtcccatcaaggttcagtggcagtggatctgggcaagattattctctcaccatcagcagcctggagaatgaagatatgggaatttattactgtctacagtatgatgagtttcccctcacgttcggtgctgggaccaagctggagctgaaacgtacggtggccgccccctccgtgtttatcttccctcccagcgacgagcagctgaagagcggcacagccagcgtggtgtgtctgctgaacaacttctaccctagagaggccaaggtgcagtggaaagtggacaacgctctgcagtccggcaacagccaagagagcgtgaccgagcaagacagcaaggacagcacctactctctgagcagcacactgacactgtccaaggccgactacgagaagcacaaggtgtacgcttgcgaggtgacccaccaaggactgagcagccccgtgaccaagagcttcaataggggcgagtgctga

SEQ ID NO: 85 - protein, 241 aa, synthetic construct
MDMRVPAQLLGLLLLWLRGARCDVLLTQIPLSLPVSLGDQASISCRASQNIVHSNGNTYLDWYLQKPGQSPKLLIYKVSNRFSGVPDRFSGSGSGTDFTLKISRVEAEDLGVYYCFQGSRLPLTFGVGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - DNA, 726 bp, synthetic construct
  source 1..726: mol_type = other DNA
atggacatgagagtgcccgcccagctgctgggactgctgctgctgtggctgagaggagcgcgctgcgatgttttgctgacccaaattccgctctccctgcctgtcagtcttggagatcaagcctccatctcctgcagagctagtcagaacattgtacatagtaatggaaacacttatttagattggtacctgcagaaaccaggccagtctccaaagctcctgatctacaaagtttccaatcgattttctggggtcccagacaggttcagtggcagtggatcagggacagatttcacactcaagatcagcagagtggaggctgaggatctgggagtttattattgttttcaaggttcacgtcttccgctcacgttcggtgttgggaccaagctggagctgaaacgtacggtggccgccccctccgtgtttatcttccctcccagcgacgagcagctgaagagcggcacagccagcgtggtgtgtctgctgaacaacttctaccctagagaggccaaggtgcagtggaaagtggacaacgctctgcagtccggcaacagccaagagagcgtgaccgagcaagacagcaaggacagcacctactctctgagcagcacactgacactgtccaaggccgactacgagaagcacaaggtgtacgcttgcgaggtgacccaccaaggactgagcagccccgtgaccaagagcttcaataggggcgagtgctga

SEQ ID NO: 87 - protein, 236 aa, synthetic construct
MDMRVPAQLLGLLLLWLRGARCDIVMTQSQKVMSTSVGDRVSVTCKASQNVGRNIAWYQQKPGQSPKTLIYSASSRYSGVPDRFTGSGFGTYFILTINNVQSEDLAAYFCQQYNSYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 88 - DNA, 711 bp, synthetic construct
  source 1..711: mol_type = other DNA
atggacatgagagtgcccgcccagctgctgggactgctgctgctgtggctgagaggagcgcgctgcgacattgtgatgacccagtctcaaaaagtcatgtccacatcagtaggagacagggtcagcgtcacctgcaaggccagtcagaatgtgggtaggaatatagcctggtatcaacagaaaccagggcaatctcctaaaacactgatatattcggcatcctcccggtacagtggagtccctgatcgcttcacaggcagtggatttgggacatatttcattctcaccatcaacaacgtgcagtctgaagacttggcagcatatttctgtcagcaatataatagttatccgtggacgttcggtggaggcaccaagctggaaatcaaacgtacggtggccgccccctccgtgtttatcttccctcccagcgacgagcagctgaagagcggcacagccagcgtggtgtgtctgctgaacaacttctaccctagagaggccaaggtgcagtggaaagtggacaacgctctgcagtccggcaacagccaagagagcgtgaccgagcaagacagcaaggacagcacctactctctgagcagcacactgacactgtccaaggccgactacgagaagcacaaggtgtacgcttgcgaggtgacccaccaaggactgagcagccccgtgaccaagagcttcaataggggcgagtgctga

SEQ ID NO: 89 - protein, 242 aa, synthetic construct
MDMRVPAQLLGLLLLWLRGARCDIVMTQSPSSLAMSIGQKVTMSCKSSQSLFNSTTHKIYLAWYQQKPGQSPKLLVYFASTRQSGVPDRFIGSGSWTDFTLTINSVQAEDLADYFCQQHYTAPWTFGGGTKLEIRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 90 - DNA, 729 bp, synthetic construct
  source 1..729: mol_type = other DNA
atggacatgagagtgcccgcccagctgctgggactgctgctgctgtggctgagaggagcgcgctgcgacattgtgatgacacagtctccatcctccctggctatgtcaattggacagaaggtcactatgagctgcaagtccagtcagagcctttttaatagtaccactcacaagatctatttggcctggtaccagcagaaaccaggacagtctcctaaacttctggtatactttgcatccactagacaatctggggtccctgatcgtttcatcggcagtggatcttggacagatttcactcttaccatcaacagtgtgcaggctgaagacctggcagattacttctgtcagcaacattataccgctccgtggacgttcggtggaggcaccaagctggaaatcagacgtacggtggccgccccctccgtgtttatcttccctcccagcgacgagcagctgaagagcggcacagccagcgtggtgtgtctgctgaacaacttctaccctagagaggccaaggtgcagtggaaagtggacaacgctctgcagtccggcaacagccaagagagcgtgaccgagcaagacagcaaggacagcacctactctctgagcagcacactgacactgtccaaggccgactacgagaagcacaaggtgtacgcttgcgaggtgacccaccaaggactgagcagccccgtgaccaagagcttcaataggggcgagtgctga

SEQ ID NO: 91 - protein, 236 aa, synthetic construct
MDMRVPAQLLGLLLLWLRGARCDIQMTQSSSSFSVSLGDRVTITCKASEDIYNRLAWYQQRPGNAPRLLISGATTLETGVPSRFSGSGSGKDYTLSITSLQTEDVATYYCQQYWSTPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 92 - DNA, 711 bp, synthetic construct
  source 1..711: mol_type = other DNA
atggacatgagagtgcccgcccagctgctgggactgctgctgctgtggctgagaggagcgcgctgcgacatccagatgacacaatcttcatcctccttttctgtatctctaggagacagagtcaccattacttgcaaggcaagtgaagacatatataatcggttagcctggtatcagcagagaccaggaaatgctcctaggctcttaatatctggtgcaaccactttggaaactggggttccttcaagattcagtggcagtggatctggaaaggattacactctcagcattaccagtcttcagactgaagatgttgctacttattactgtcaacaatattggagtactccgtggacgttcggtggaggcaccaagctggaaatcaaacgtacggtggccgccccctccgtgtttatcttccctcccagcgacgagcagctgaagagcggcacagccagcgtggtgtgtctgctgaacaacttctaccctagagaggccaaggtgcagtggaaagtggacaacgctctgcagtccggcaacagccaagagagcgtgaccgagcaagacagcaaggacagcacctactctctgagcagcacactgacactgtccaaggccgactacgagaagcacaaggtgtacgcttgcgaggtgacccaccaaggactgagcagccccgtgaccaagagcttcaataggggcgagtgctga

SEQ ID NO: 93 - protein, 241 aa, synthetic construct
MDMRVPAQLLGLLLLWLRGARCDVLMTQTPLSLPVSLGDQASISCRSSQNIVHSNGDTYLEWYLQKPGQSPKLLIYRVSNRFSGVPDRFSGSGSGTDFTLKISRVEAEDLGVYYCFQGSHVPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - DNA, 726 bp, synthetic construct
  source 1..726: mol_type = other DNA
atggacatgagagtgcccgcccagctgctgggactgctgctgctgtggctgagaggagcgcgctgcgatgttttgatgacccaaactccactctccctgcctgtcagtcttggagatcaagcctccatctcttgcagatctagtcagaacattgtacatagtaatggagacacctatttagaatggtacctgcagaaaccaggccagtctccaaagctcctgatctacagagtttccaaccgattttctggggtcccagacaggttcagtggcagtggatcagggacagatttcacactcaagatcagcagagtggaggctgaggatctgggagtttattactgctttcaaggttcacatgttccgctcacgttcggtgctgggaccaagctggagctgaaacgtacggtggccgccccctccgtgtttatcttccctcccagcgacgagcagctgaagagcggcacagccagcgtggtgtgtctgctgaacaacttctaccctagagaggccaaggtgcagtggaaagtggacaacgctctgcagtccggcaacagccaagagagcgtgaccgagcaagacagcaaggacagcacctactctctgagcagcacactgacactgtccaaggccgactacgagaagcacaaggtgtacgcttgcgaggtgacccaccaaggactgagcagccccgtgaccaagagcttcaataggggcgagtgctga

SEQ ID NO: 95 - protein, 241 aa, synthetic construct
MDMRVPAQLLGLLLLWLRGARCDVLMTQTPLSLPVSLGDQASISCRSSQAIVHSNGNTYLEWFLQKPGQSPKLLVYKVSNRFSGVPDRFSGSGSGTDFTLRISRVEAEDLGVYYCFQSSHIPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - DNA, 726 bp, synthetic construct
  source 1..726: mol_type = other DNA
atggacatgagagtgcccgcccagctgctgggactgctgctgctgtggctgagaggagcgcgctgcgatgttttgatgacccagactccactctccctgcctgtcagtcttggagatcaagcctccatctcgtgcagatctagtcaggccattgtacatagtaatggaaacacctatttagaatggttcctgcagaaaccaggccagtctccaaagctcctggtctacaaagtttccaaccgattttctggggtcccagacaggttcagtggcagtggatcagggacagatttcacactcaggatcagcagagtggaggctgaggatctgggagtttattactgctttcagagttctcatattccgtggacgttcggtggaggcaccaagctggaaatcaaacgtacggtggccgccccctccgtgtttatcttccctcccagcgacgagcagctgaagagcggcacagccagcgtggtgtgtctgctgaacaacttctaccctagagaggccaaggtgcagtggaaagtggacaacgctctgcagtccggcaacagccaagagagcgtgaccgagcaagacagcaaggacagcacctactctctgagcagcacactgacactgtccaaggccgactacgagaagcacaaggtgtacgcttgcgaggtgacccaccaaggactgagcagccccgtgaccaagagcttcaataggggcgagtgctga

SEQ ID NO: 97 - protein, 464 aa, synthetic construct
MEFGLSWVFLVAILKGVQCEVKLVESGGGLVQPGGSLRLSCATSGFTFTDYYMSWVRQPPGKALEWLGFIRNKANGYRTEYSASVKGRFTISRDNSQSILYLQMNTLRAEDSATYYCASLSAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - DNA, 1395 bp, synthetic construct
  source 1..1395: mol_type = other DNA
atggagttcggactgagctgggtgtttctggtggccattctgaagggtgtacagtgcgaggtgaagctggtggagtctggaggaggcttggtacagcctgggggttctctgagactctcctgtgcaacttctgggttcaccttcactgattactacatgagctgggtccgccagcctccaggaaaggcacttgagtggttgggttttattagaaacaaagctaatggttacagaacagagtacagtgcatctgtgaagggtcggttcaccatctccagagataattcccaaagcatcctctatcttcaaatgaacaccctgagagctgaggacagtgccacttattactgtgcaagcttatctgcttactggggccaagggactctggtcactgtctctgcagctagcaccaagggccctagcgtgtttcctctggccccctccagcaagagcacaagcggaggaaccgctgctctgggatgtctggtgaaggattacttccccgagcccgtgacagtgagctggaacagcggagctctgacctccggcgtgcatacatttcccgccgtgctgcagagcagcggcctctactctctgtccagcgtggtgaccgtgcctagctcctctctgggaacacagacctacatctgcaacgtgaaccacaagccctccaacaccaaggtcgataagaaggtggagcccaaaagctgcgacaagacacacacatgtcccccttgccccgctcccgagctgctgggaggaccctccgtgtttctgtttccccccaagcccaaggacacactgatgatcagcagaacacccgaggtgacatgcgtggtggtggatgtcagccacgaggaccccgaggtcaagttcaattggtacgtggacggcgtggaggtgcacaatgccaagaccaagcccagagaggagcagtacaactccacatatagagtggtgagcgtgctgaccgtgctgcaccaagactggctgaacggcaaggaatacaagtgcaaggtctccaacaaggctctgcccgcccccatcgagaagacaatctccaaggccaaaggccagcccagagagccccaagtctacacactgcctccctctagggatgagctgaccaagaaccaagtgagcctcacatgtctggtgaagggcttctatcccagcgacatcgctgtggagtgggagagcaacggccagcccgagaacaactacaagaccacaccccccgtgctggacagcgatggctccttctttctgtactccaagctgaccgtggacaaatctaggtggcaacaaggcaacgtgtttagctgctccgtgatgcacgaggccctccacaaccactatacccagaagtctctgtctctgtcccccggcaagtga

SEQ ID NO: 99 - protein, 462 aa, synthetic construct
MEFGLSWVFLVAILKGVQCEVQLQESGPSLVKPSQTLSLTCSVTGASITSGHWNWIRKFPGNKLEYMGYIRDSGTTYYNPSLKGRLSITRDTSKNQYYLQLSSVTAEDTATYYCVTTPFVYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - DNA, 1389 bp, synthetic construct
  source 1..1389: mol_type = other DNA
atggagttcggactgagctgggtgtttctggtggccattctgaagggtgtacagtgcgaggtgcagcttcaggagtcaggacctagcctcgtgaaaccttctcagactctgtccctcacctgttctgtcactggcgcctccatcaccagtggtcactggaactggatccggaaattccctggaaataaacttgaatatatggggtacataagggacagtggtaccacttactacaacccatctctcaaaggtcgactctccatcactcgagacacatccaagaaccagtactacctgcagttgagttctgtgactgctgaggacacagccacatattattgtgtgactacgccgtttgtttactggggccaagggactctggtcactgtctctgcagctagcaccaagggccctagcgtgtttcctctggccccctccagcaagagcacaagcggaggaaccgctgctctgggatgtctggtgaaggattacttccccgagcccgtgacagtgagctggaacagcggagctctgacctccggcgtgcatacatttcccgccgtgctgcagagcagcggcctctactctctgtccagcgtggtgaccgtgcctagctcctctctgggaacacagacctacatctgcaacgtgaaccacaagccctccaacaccaaggtcgataagaaggtggagcccaaaagctgcgacaagacacacacatgtcccccttgccccgctcccgagctgctgggaggaccctccgtgtttctgtttccccccaagcccaaggacacactgatgatcagcagaacacccgaggtgacatgcgtggtggtggatgtcagccacgaggaccccgaggtcaagttcaattggtacgtggacggcgtggaggtgcacaatgccaagaccaagcccagagaggagcagtacaactccacatatagagtggtgagcgtgctgaccgtgctgcaccaagactggctgaacggcaaggaatacaagtgcaaggtctccaacaaggctctgcccgcccccatcgagaagacaatctccaaggccaaaggccagcccagagagccccaagtctacacactgcctccctctagggatgagctgaccaagaaccaagtgagcctcacatgtctggtgaagggcttctatcccagcgacatcgctgtggagtgggagagcaacggccagcccgagaacaactacaagaccacaccccccgtgctggacagcgatggctccttctttctgtactccaagctgaccgtggacaaatctaggtggcaacaaggcaacgtgtttagctgctccgtgatgcacgaggccctccacaaccactatacccagaagtctctgtctctgtcccccggcaagtga

SEQ ID NO: 101 - protein, 461 aa, synthetic construct
MEFGLSWVFLVAILKGVQCQVHLKESGPGLVAPSQSLSITCTVSGFSLFNYGVHWVRQPPGKGLEWLGVIWADGTINYNSALMSRLTITKDNIKSQVFLKMDSLKTDDTAIYYCARHFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - DNA, 1386 bp, synthetic construct
  source 1..1386: mol_type = other DNA
atggagttcggactgagctgggtgtttctggtggccattctgaagggtgtacagtgccaggtgcacctgaaggagtcaggacctggcctggtggcgccctcacagagcctgtccatcacttgcactgtctctggcttttcattattcaactatggtgtacactgggttcgccagcctccaggaaagggtctggagtggctgggagttatttgggctgacggaaccataaattataattcggctctcatgtccagactgaccatcaccaaagacaacatcaagagccaggttttcttaaaaatggacagtctgaaaactgacgacacagccatatactactgtgccagacactttgactactggggccaaggcaccactctcacagtctcctcagctagcaccaagggccctagcgtgtttcctctggccccctccagcaagagcacaagcggaggaaccgctgctctgggatgtctggtgaaggattacttccccgagcccgtgacagtgagctggaacagcggagctctgacctccggcgtgcatacatttcccgccgtgctgcagagcagcggcctctactctctgtccagcgtggtgaccgtgcctagctcctctctgggaacacagacctacatctgcaacgtgaaccacaagccctccaacaccaaggtcgataagaaggtggagcccaaaagctgcgacaagacacacacatgtcccccttgccccgctcccgagctgctgggaggaccctccgtgtttctgtttccccccaagcccaaggacacactgatgatcagcagaacacccgaggtgacatgcgtggtggtggatgtcagccacgaggaccccgaggtcaagttcaattggtacgtggacggcgtggaggtgcacaatgccaagaccaagcccagagaggagcagtacaactccacatatagagtggtgagcgtgctgaccgtgctgcaccaagactggctgaacggcaaggaatacaagtgcaaggtctccaacaaggctctgcccgcccccatcgagaagacaatctccaaggccaaaggccagcccagagagccccaagtctacacactgcctccctctagggatgagctgaccaagaaccaagtgagcctcacatgtctggtgaagggcttctatcccagcgacatcgctgtggagtgggagagcaacggccagcccgagaacaactacaagaccacaccccccgtgctggacagcgatggctccttctttctgtactccaagctgaccgtggacaaatctaggtggcaacaaggcaacgtgtttagctgctccgtgatgcacgaggccctccacaaccactatacccagaagtctctgtctctgtcccccggcaagtga

SEQ ID NO: 103 - protein, 468 aa, synthetic construct
MEFGLSWVFLVAILKGVQCDVKLVESGGGLVKLGGSLKLSCAASGFIFSSYYMSWVRQTPEKKLELVAAININGGSTYYPDTVKGRFTISRDNAKNTLYLQMSSLKSEDTALYYCGRHDHGYGWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - DNA, 1407 bp, synthetic construct
  source 1..1407: mol_type = other DNA
atggagttcggactgagctgggtgtttctggtggccattctgaagggtgtacagtgcgacgtgaagctcgtggagtcggggggaggcttagtgaaacttggagggtccctgaaactctcctgtgcagcctctggattcattttcagtagctattacatgtcttgggttcgccagactccggagaagaagctggagttggtcgcagccattaatattaatggtggtagcacctactatccagacactgtgaagggccgattcaccatctccagagacaatgccaagaacaccctgtacctgcaaatgagcagtctgaagtctgaggacacagccttatattactgtggaagacatgaccatgggtacggctggtttgcttactggggccaagggactctggtcactgtctctgcagctagcaccaagggccctagcgtgtttcctctggccccctccagcaagagcacaagcggaggaaccgctgctctgggatgtctggtgaaggattacttccccgagcccgtgacagtgagctggaacagcggagctctgacctccggcgtgcatacatttcccgccgtgctgcagagcagcggcctctactctctgtccagcgtggtgaccgtgcctagctcctctctgggaacacagacctacatctgcaacgtgaaccacaagccctccaacaccaaggtcgataagaaggtggagcccaaaagctgcgacaagacacacacatgtcccccttgccccgctcccgagctgctgggaggaccctccgtgtttctgtttccccccaagcccaaggacacactgatgatcagcagaacacccgaggtgacatgcgtggtggtggatgtcagccacgaggaccccgaggtcaagttcaattggtacgtggacggcgtggaggtgcacaatgccaagaccaagcccagagaggagcagtacaactccacatatagagtggtgagcgtgctgaccgtgctgcaccaagactggctgaacggcaaggaatacaagtgcaaggtctccaacaaggctctgcccgcccccatcgagaagacaatctccaaggccaaaggccagcccagagagccccaagtctacacactgcctccctctagggatgagctgaccaagaaccaagtgagcctcacatgtctggtgaagggcttctatcccagcgacatcgctgtggagtgggagagcaacggccagcccgagaacaactacaagaccacaccccccgtgctggacagcgatggctccttctttctgtactccaagctgaccgtggacaaatctaggtggcaacaaggcaacgtgtttagctgctccgtgatgcacgaggccctccacaaccactatacccagaagtctctgtctctgtcccccggcaagtga

SEQ ID NO: 105 - protein, 466 aa, synthetic construct
MEFGLSWVFLVAILKGVQCQIQLVQSGPELKNPGETVRISCKASGYTFTTAGIQWVQKMPGKGLKWIGWINTQSGVSKCAEDFKGRFALSMETYASTAYLQMSNLKKEDTATYFCARSNSGGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - DNA, 1401 bp, synthetic construct
  source 1..1401: mol_type = other DNA
atggagttcggactgagctgggtgtttctggtggccattctgaagggtgtacagtgccaaatccagttggtgcagtcaggacctgagctgaagaaccctggagagacagtcaggatctcctgcaaggcttctgggtataccttcacaactgctggaatccagtgggtgcaaaagatgccaggaaagggtttgaagtggattggctggataaacacccagtctggagtgtcaaaatgtgcagaagacttcaagggtcgatttgccttgtccatggaaacctacgccagcactgcatatttacagatgagcaacctcaaaaaagaggacacggctacgtatttctgtgcgagatcgaactccggcggttttccttactggggccaagggactctggtcactgtctctgcagctagcaccaagggccctagcgtgtttcctctggccccctccagcaagagcacaagcggaggaaccgctgctctgggatgtctggtgaaggattacttccccgagcccgtgacagtgagctggaacagcggagctctgacctccggcgtgcatacatttcccgccgtgctgcagagcagcggcctctactctctgtccagcgtggtgaccgtgcctagctcctctctgggaacacagacctacatctgcaacgtgaaccacaagccctccaacaccaaggtcgataagaaggtggagcccaaaagctgcgacaagacacacacatgtcccccttgccccgctcccgagctgctgggaggaccctccgtgtttctgtttccccccaagcccaaggacacactgatgatcagcagaacacccgaggtgacatgcgtggtggtggatgtcagccacgaggaccccgaggtcaagttcaattggtacgtggacggcgtggaggtgcacaatgccaagaccaagcccagagaggagcagtacaactccacatatagagtggtgagcgtgctgaccgtgctgcaccaagactggctgaacggcaaggaatacaagtgcaaggtctccaacaaggctctgcccgcccccatcgagaagacaatctccaaggccaaaggccagcccagagagccccaagtctacacactgcctccctctagggatgagctgaccaagaaccaagtgagcctcacatgtctggtgaagggcttctatcccagcgacatcgctgtggagtgggagagcaacggccagcccgagaacaactacaagaccacaccccccgtgctggacagcgatggctccttctttctgtactccaagctgaccgtggacaaatctaggtggcaacaaggcaacgtgtttagctgctccgtgatgcacgaggccctccacaaccactatacccagaagtctctgtctctgtcccccggcaagtga

SEQ ID NO: 107 - protein, 467 aa, synthetic construct
MEFGLSWVFLVAILKGVQCQIQLVQSGPELKKPGETVRISCKASGYTFTTVGMQWVQKMPGKGLRWIGWINTHSGVSKYAEDFKGRFAFSLETSASTAYLEISNLKNEDTATYFCARGNSNYGFPYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - DNA, 1404 bp, synthetic construct
  source 1..1404: mol_type = other DNA
atggagttcggactgagctgggtgtttctggtggccattctgaagggtgtacagtgccagatccagttggtgcagtctggacctgagctgaagaagcctggagagacagtcaggatctcctgcaaggcttctgggtataccttcacaactgttggaatgcagtgggtgcaaaagatgccaggaaagggtttgaggtggattggctggataaacacccactctggagtgtcaaaatatgcagaagacttcaagggacggtttgccttctctttggaaacctctgccagcactgcatatttagagataagcaacctcaaaaatgaggacacggctacgtatttctgtgcgaggggaaatagtaactacgggtttccttactggggccaagggactctggtcactgtctctgcagctagcaccaagggccctagcgtgtttcctctggccccctccagcaagagcacaagcggaggaaccgctgctctgggatgtctggtgaaggattacttccccgagcccgtgacagtgagctggaacagcggagctctgacctccggcgtgcatacatttcccgccgtgctgcagagcagcggcctctactctctgtccagcgtggtgaccgtgcctagctcctctctgggaacacagacctacatctgcaacgtgaaccacaagccctccaacaccaaggtcgataagaaggtggagcccaaaagctgcgacaagacacacacatgtcccccttgccccgctcccgagctgctgggaggaccctccgtgtttctgtttccccccaagcccaaggacacactgatgatcagcagaacacccgaggtgacatgcgtggtggtggatgtcagccacgaggaccccgaggtcaagttcaattggtacgtggacggcgtggaggtgcacaatgccaagaccaagcccagagaggagcagtacaactccacatatagagtggtgagcgtgctgaccgtgctgcaccaagactggctgaacggcaaggaatacaagtgcaaggtctccaacaaggctctgcccgcccccatcgagaagacaatctccaaggccaaaggccagcccagagagccccaagtctacacactgcctccctctagggatgagctgaccaagaaccaagtgagcctcacatgtctggtgaagggcttctatcccagcgacatcgctgtggagtgggagagcaacggccagcccgagaacaactacaagaccacaccccccgtgctggacagcgatggctccttctttctgtactccaagctgaccgtggacaaatctaggtggcaacaaggcaacgtgtttagctgctccgtgatgcacgaggccctccacaaccactatacccagaagtctctgtctctgtcccccggcaagtga

SEQ ID NO: 109 - protein, 465 aa, synthetic construct
MEFGLSWVFLVAILKGVQCDVQLQESGPGLVKPSQSLSLTCTVTGYSITSDYAWTWIRQFPGNKLEWMGYISYRSITGYNPSLKSRISINRDTSKNQFFLQLNSVTTEDTATYYCARFGYDGNYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - DNA, 1398 bp, synthetic construct
  source 1..1398: mol_type = other DNA
atggagttcggactgagctgggtgtttctggtggccattctgaagggtgtacagtgcgatgtgcagcttcaggagtcgggacctggcctggtgaaaccttctcagtctctgtccctcacctgcactgtcactggctactcaatcaccagtgattatgcctggacctggatccggcagtttcccggaaacaaactggagtggatgggctacataagctacagaagtatcactggctacaacccatctctcaaaagtcgaatttctatcaatcgagacacatccaagaaccagttcttcctgcagttgaattctgtgactactgaggacacagccacatattactgtgcaagatttggttatgacgggaactactggggccaaggcaccactctcacagtctcctcagctagcaccaagggccctagcgtgtttcctctggccccctccagcaagagcacaagcggaggaaccgctgctctgggatgtctggtgaaggattacttccccgagcccgtgacagtgagctggaacagcggagctctgacctccggcgtgcatacatttcccgccgtgctgcagagcagcggcctctactctctgtccagcgtggtgaccgtgcctagctcctctctgggaacacagacctacatctgcaacgtgaaccacaagccctccaacaccaaggtcgataagaaggtggagcccaaaagctgcgacaagacacacacatgtcccccttgccccgctcccgagctgctgggaggaccctccgtgtttctgtttccccccaagcccaaggacacactgatgatcagcagaacacccgaggtgacatgcgtggtggtggatgtcagccacgaggaccccgaggtcaagttcaattggtacgtggacggcgtggaggtgcacaatgccaagaccaagcccagagaggagcagtacaactccacatatagagtggtgagcgtgctgaccgtgctgcaccaagactggctgaacggcaaggaatacaagtgcaaggtctccaacaaggctctgcccgcccccatcgagaagacaatctccaaggccaaaggccagcccagagagccccaagtctacacactgcctccctctagggatgagctgaccaagaaccaagtgagcctcacatgtctggtgaagggcttctatcccagcgacatcgctgtggagtgggagagcaacggccagcccgagaacaactacaagaccacaccccccgtgctggacagcgatggctccttctttctgtactccaagctgaccgtggacaaatctaggtggcaacaaggcaacgtgtttagctgctccgtgatgcacgaggccctccacaaccactatacccagaagtctctgtctctgtcccccggcaagtga

SEQ ID NO: 111 - protein, 462 aa, synthetic construct
MEFGLSWVFLVAILKGVQCEVLLQQSGPELVKPGTSVKIPCKASGYTFTDYNMDWVKQSHGKSLEWIGDINPDNGATVYNQKFKGKATLTVDKSSSTAYMELRSLTSEDTAVYYCVRGFDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - DNA, 1389 bp, synthetic construct
  source 1..1389: mol_type = other DNA
atggagttcggactgagctgggtgtttctggtggccattctgaagggtgtacagtgcgaggtcctgctgcaacagtctggacctgagctggtgaagcctgggacttcagtgaagataccctgcaaggcttctggatacacattcactgactacaacatggactgggtgaagcagagccatggaaagagccttgagtggattggagatattaatcctgacaatggtgctactgtctacaaccagaagttcaagggcaaggccacattgactgttgacaagtcctccagcacagcctacatggagctccgcagcctgacatctgaggacactgcagtctattactgtgtaagagggttcgactactggggtcaaggaacctcagtcaccgtctcctcagctagcaccaagggccctagcgtgtttcctctggccccctccagcaagagcacaagcggaggaaccgctgctctgggatgtctggtgaaggattacttccccgagcccgtgacagtgagctggaacagcggagctctgacctccggcgtgcatacatttcccgccgtgctgcagagcagcggcctctactctctgtccagcgtggtgaccgtgcctagctcctctctgggaacacagacctacatctgcaacgtgaaccacaagccctccaacaccaaggtcgataagaaggtggagcccaaaagctgcgacaagacacacacatgtcccccttgccccgctcccgagctgctgggaggaccctccgtgtttctgtttccccccaagcccaaggacacactgatgatcagcagaacacccgaggtgacatgcgtggtggtggatgtcagccacgaggaccccgaggtcaagttcaattggtacgtggacggcgtggaggtgcacaatgccaagaccaagcccagagaggagcagtacaactccacatatagagtggtgagcgtgctgaccgtgctgcaccaagactggctgaacggcaaggaatacaagtgcaaggtctccaacaaggctctgcccgcccccatcgagaagacaatctccaaggccaaaggccagcccagagagccccaagtctacacactgcctccctctagggatgagctgaccaagaaccaagtgagcctcacatgtctggtgaagggcttctatcccagcgacatcgctgtggagtgggagagcaacggccagcccgagaacaactacaagaccacaccccccgtgctggacagcgatggctccttctttctgtactccaagctgaccgtggacaaatctaggtggcaacaaggcaacgtgtttagctgctccgtgatgcacgaggccctccacaaccactatacccagaagtctctgtctctgtcccccggcaagtga

SEQ ID NO: 113 - protein, 601 aa, synthetic construct
ETFSGGCLFDEPYSTCGYSQSEGDDFNWEQVNTLTKPTSDPWMPSGSFMLVNASGRPEGQRAHLLLPQLKENDTHCIDFHYFVSSKSNSPPGLLNVYVKVNNGPLGNPIWNISGDPTRTWNRAELAISTFWPNFYQVIFEVITSGHQGYLAIDEVKVLGHPCTRTPHFLRIQNVEVNAGQFATFQCSAIGRTVAGDRLWLQGIDVRDAPLKEIKVTSSRRFIASFNVVNTTKRDAGKYRCMIRTEGGVGISNYAELVVKEPPVPIAPPQLASVGATYLWIQLNANSINGDGPIVAREVEYCTASGSWNDRQPVDSTSYKIGHLDPDTEYEISVLLTRPGEGGTGSPGPALRTRTKCADPMRGPRKLEVVEVKSRQITIRWEPFGYNVTRCHSYNLTVHYCYQVGGQEQVREEVSWDTENSHPQHTITNLSPYTNVSVKLILMNPEGRKESQELIVQTDEDLPGAVPTESIQGSTFEEKIFLQWREPTQTYGVITLYEITYKAVSSFDPEIDLSNQSGRVSKLGNETHFLFFGLYPGTTYSFTIRASTAKGFGPPATNQFTTKISAPSMPAYELETPLNQTDNTVTVMLKPAHSRGAPVSVY

SEQ ID NO: 114 - DNA, 1803 bp, synthetic construct
  source 1..1803: mol_type = other DNA
gagacgttctcaggtggctgcctctttgatgagccgtatagcacatgtggatatagtcaatctgaaggtgatgacttcaattgggagcaagtgaacaccttgactaaaccgacttctgatccatggatgccatcaggttctttcatgctggtgaatgcctctgggagacctgaggggcagagagcccacctgctcttaccccaacttaaagaaaatgacacccactgcatcgattttcactattttgtgtccagcaagagtaattctcctccggggttactcaatgtctacgtgaaggtcaataacgggccactggggaatcctatctggaatatatctggagacccaacacgtacatggaacagggcagaactggccattagtactttctggcctaacttttatcaggtgatttttgaagtgataacttctggacatcaaggctatctcgctatcgatgaggtgaaggtgttaggacatccatgtaccaggactcctcacttcctgcggattcagaatgtggaagttaatgctggccagtttgctaccttccagtgcagtgccatcggcaggaccgtggcaggagacaggctctggttacagggcattgatgtgcgagatgctcctctgaaggaaatcaaggtgaccagctcccgacgcttcattgcttcatttaatgttgtgaataccaccaaacgagatgctggaaagtaccgctgcatgattcgcactgaaggaggtgttggaatatcaaactatgcagagttggtagttaaagaaccacccgttcctattgccccacctcagctcgcctctgtaggagccacctacctgtggatacagctcaacgccaactccatcaatggggatgggcccattgtggcccgagaggtggagtactgcacggccagtgggagctggaatgaccggcagccagtcgattccacgagctataaaattggacaccttgacccagatacagaatatgagattagtgtgctcctgaccaggccaggggagggtggcactggctctcctggtccagctctcaggacaagaacaaagtgtgctgatcccatgcgaggcccaagaaaactagaagtagtggaggtcaaatctcggcaaatcactatccgctgggagccatttggatataatgtaactcgttgccacagttataatctcactgtccactactgttaccaagttggaggacaagaacaagtgcgagaagaagtaagctgggatacagaaaattcacaccctcaacacacgatcactaacctgtcaccatacaccaatgtcagtgtgaaactgatcctcatgaacccagagggccggaaggaaagccaagaactcatagtgcagacagatgaagacctcccaggtgctgttcccactgaatccatacaaggaagtacctttgaagagaagatatttcttcagtggagagaaccaactcaaacatatggtgtaatcactttatatgagatcacctacaaagcagtcagttcctttgacccagaaatagatttatccaatcagagtggaagagtttcaaagctgggaaatgaaacccattttctgttttttggactgtatccggggaccacatactcctttaccatccgagctagcacagctaagggttttgggcctccagcaacaaaccagttcaccaccaaaatatcagcaccctctatgccagcttatgaacttgagacacctttgaatcaaactgacaataccgtgacagtcatgctgaaacctgcccacagcagaggagcacctgtcagtgtctat

(19) United States
(12) Patent Application Publication
(10) Pub. No.: US 2026/0242514 A1
(43) Pub. Date: Aug. 20, 2026

(54) CELL ADHESION MOLECULE TARGETED IMMUNOTHERAPIES

(71) Applicant: CASE WESTERN RESERVE UNIVERSITY, Cleveland, OH (US)

(72) Inventors: Susann Brady-Kalnay, Wellington, OH (US); Cynthia Hale, Cleveland, OH (US)

(21) Appl. No.: 19/161,896

(22) PCT Filed: Mar. 4, 2024
(86) PCT No.: PCT/US2024/018356

§ 371 Date: Sep. 3, 2025

Related U.S. Application Data
(60) Provisional application No. 63/488,221, filed on Mar. 3, 2023.

(51) Int. Cl. C07K 16/40; A61K 38/00; C07K 14/705

(52) U.S. Cl. CPC C07K 16/40; C07K 14/7051; A61K 38/00; C07K 2317/24; C07K 2317/622

(57) ABSTRACT

A peptide, protein, antibody, antibody fragment, or antigen binding fragment thereof to a cell adhesion molecule for use as an immunotherapy.

### RELATED APPLICATION

This application claims priority from U.S. Provisional Application No. 63/488,221, filed Mar. 3, 2023, the subject matter of which is incorporated herein by reference in its entirety.

### SEQUENCE LISTING

The instant application contains a Sequence Listing which has been submitted electronically in XML format and is hereby incorporated by reference in its entirety. Said XML copy, created on Feb. 23, 2024, is named CWR-032502WO ORD.st.26 and is 126,180 bytes in size.

### FIELD OF THE INVENTION

This disclosure relates to cell adhesion molecule (CAM) targeted immunotherapies and, particularly, peptides, proteins, antibodies, antibody fragments, and antigen-binding fragments thereof that bind to CAMs and their use in immunotherapies.

### BACKGROUND

Brain tumors are one of the deadliest forms of cancers. This includes both primary brain cancer and tumors that metastasize to the brain. The highest-grade brain cancer, grade 4 glioma or glioblastoma (GBM), has a median survival of one year. The dispersive and invasive cells characteristic of GBM contribute substantially to poor survival associated with this disease. Unfortunately, there are no effective treatments, and novel therapies that target these invasive tumors are required. Furthermore, tumors that metastasize to the brain also have very poor outcomes.

### SUMMARY

Embodiments described herein relate to cell adhesion molecule (CAM) targeted immunotherapies and, particularly, peptides, proteins, antibodies, antibody fragments, and antigen-binding fragments thereof that bind to CAMs and their use in immunotherapies. Proteolysis of CAMs is a common event in cancer and cleaved fragments of CAMs can function as tumor biomarkers. Full length CAMs including the Ig superfamily receptor protein tyrosine phosphatase, PTPμ, function as tumor suppressors and normally contribute to cell-cell adhesion by binding homophilically to PTPμ on adjacent cells to link cells together. In the microenvironment of multiple tumor types, PTPμ proteolysis creates an extracellular domain (ECD) fragment that leads to the formation of unique epitopes (neoantigens) expressed exclusively on the surface of tumor cells.

We developed immunotherapies based on peptides, proteins, antibodies, antibody fragments thereof, and antigen binding fragments thereof that bind to CAMs, such as PTPμ. The peptides, proteins, antibodies, antibody fragments, and antigen binding fragments thereof can include peptides and proteins that bind to CAMs, such as PTPμ, humanized and chimeric versions of antibodies, antibody fragments thereof, and antigen binding fragments that bind to CAMs, such as PTPμ, scFv antibody fragments that bind to CAMs, such as PTPμ, as well as Fc domain chimeric proteins, in which the Fc domain of the human immunoglobulin heavy chain IgG1 is conjugated to a tumor specific protein that binds to CAMs, such as PTPμ.

The Fc domain binds to Fcγ receptors (FcγR) on innate immune cells triggering antibody-dependent cellular cytotoxicity (ADCC) by natural killer (NK) cells and phagocytosis of tumor cells by macrophages in the periphery or microglia in the brain. Additionally, the FcγR on dendritic cells (DC) present tumor antigens to T cells.

In some embodiments, the peptide, protein, antibody, antibody fragment, or antigen binding fragment thereof can include a PTPμ-Fc chimeric agent that stimulates the immune system to target and kill tumor cells. By combining the tumor targeting potential of PTPμ with the body's own immune recognition, the PTPμ-Fc chimeric agent can trigger ADCC.

In other embodiments, the peptide, protein, antibody, antibody fragment, or antigen binding fragment thereof can include humanized and chimeric antibodies, scFvs and peptide sequences targeting PTPμ that can be used as immunotherapies.

In some embodiments, an antibody, antibody fragment or antigen binding fragment thereof can include at least one of:
a)
a CDR-H1 comprising the amino acid sequence of GFTFTDYY (SEQ ID NO: 3),
a CDR-H2 comprising the amino acid sequence of IRNKANGYRT (SEQ ID NO: 4), and
a CDR-H3 comprising the amino acid sequence of ASLSAY (SEQ ID NO: 5); b)
a CDR-L1 comprising the amino acid sequence of KSLLHSNGITY (SEQ ID NO: 8),
a CDR-L2 comprising the amino acid sequence of QMS (SEQ ID NO: 9), and
a CDR-L3 comprising the amino acid sequence of AQNLELWT (SEQ ID NO: 10);
c)
a CDR-H1 comprising the amino acid sequence of GASITSGH (SEQ ID NO: 13),
a CDR-H2 comprising the amino acid sequence of IRDSGTT (SEQ ID NO: 14), and
a CDR-H3 comprising the amino acid sequence of VTTPFVY (SEQ ID NO: 15);
d)
a CDR-L1 comprising the amino acid sequence of QDINSY (SEQ ID NO: 18),
a CDR-L2 comprising the amino acid sequence of RAN (SEQ ID NO: 19), and
a CDR-L3 comprising the amino acid sequence of LQYDEFPLT (SEQ ID NO: 20);
e)
a CDR-H1 comprising the amino acid sequence of GFSLFNYG (SEQ ID NO: 23),
a CDR-H2 comprising the amino acid sequence of IWADGTI (SEQ ID NO: 24), and
a CDR-H3 comprising the amino acid sequence of ARHFDY (SEQ ID NO: 25);
f)
a CDR-L1 comprising the amino acid sequence of QNIVHSNGNTY (SEQ ID NO: 28),
a CDR-L2 comprising the amino acid sequence of KVS (SEQ ID NO: 29), and
a CDR-L3 comprising FQGSRLPLT (SEQ ID NO: 30);
g)
a CDR-H1 comprising the amino acid sequence of GFIFSSYY (SEQ ID NO: 33),
a CDR-H2 comprising the amino acid sequence of ININGGST (SEQ ID NO: 34), and
a CDR-H3 comprising the amino acid sequence of GRHDHGYGWFAY (SEQ ID NO: 35);
h)
a CDR-L1 comprising the amino acid sequence of QNVGRN (SEQ ID NO: 38),
a CDR-L2 comprising the amino acid sequence of SAS (SEQ ID NO: 39), and
a CDR-L3 comprising the amino acid sequence of QQYNSYPWT (SEQ ID NO: 40);
i)
a CDR-H1 comprising the amino acid sequence of GYTFTTAG (SEQ ID NO: 43),
a CDR-H2 comprising the amino acid sequence of INTQSGVS (SEQ ID NO: 44), and
a CDR-H3 comprising the amino acid sequence of ARSNSGGFPY (SEQ ID NO: 45);
j)
a CDR-L1 comprising the amino acid sequence QSLFNSTTHKIY (SEQ ID NO: 48),
a CDR-L2 comprising the amino acid sequence FAS (SEQ ID NO: 49), and
a CDR-L3 comprising the amino acid sequence of QQHYTAPWT (SEQ ID NO: 50);
k)
a CDR-H1 comprising the amino acid sequence of GYTFTTVG (SEQ ID NO: 53),
a CDR-H2 comprising the amino acid sequence of INTHSGVS (SEQ ID NO: 54), and
a CDR-H3 comprising the amino acid sequence of ARGNSNYGFPY (SEQ ID NO: 55);
l)
a CDR-L1 comprising the amino acid sequence of EDIYNR (SEQ ID NO: 58),
a CDR-L2 comprising the amino acid sequence of GAT (SEQ ID NO: 59), and
a CDR-L3 comprising the amino acid sequence of QQYWSTPWT (SEQ ID NO: 60);
m)
a CDR-H1 comprising the amino acid sequence of GYSITSDYA (SEQ ID NO: 63),
a CDR-H2 comprising the amino acid sequence of ISYRSIT (SEQ ID NO: 64), and
a CDR-H3 comprising the amino acid sequence of ARFGYDGNY (SEQ ID NO: 65);
n)
a CDR-L1 comprising the amino acid sequence of QNIVHSNGDTY (SEQ ID NO: 68),
a CDR-L2 comprising the amino acid sequence of RVS (SEQ ID NO: 69), and
a CDR-L3 comprising the amino acid sequence of FQGSHVPLT (SEQ ID NO: 70);
o)
a CDR-H1 comprising the amino acid sequence of GYTFTDYN (SEQ ID NO: 73),
a CDR-H2 comprising the amino acid sequence of INPDNGAT (SEQ ID NO: 74), and
a CDR-H3 comprising the amino acid sequence of VRGFDY (SEQ ID NO: 75);
p)
a CDR-L1 comprising the amino acid sequence of QAIVHSNGNTY (SEQ ID NO: 78), a CDR-L2 comprising the amino acid sequence of KVS (SEQ ID NO: 79), and
a CDR-L3 comprising the amino acid sequence of FQSSHIPWT (SEQ ID NO: 80); or
a heavy chain variable region and/or light chain variable region that competitively inhibits binding of a peptide, protein, isolated antibody, antibody fragment, or antigen binding fragment thereof comprising at least one of a), b), c), d), e), f), g), h), i), j), k), l), m), n), o), or p) to a CAM, such as PTPμ.

In some embodiments, the antibody, antibody fragment, or antigen binding fragment thereof can include a heavy chain variable region that includes the 3 CDRs of one of SEQ ID NO: 1, SEQ ID NO: 11, SEQ ID NO: 21, SEQ ID NO: 31, SEQ ID NO: 41, SEQ ID NO: 51, SEQ ID NO: 61, or SEQ ID NO: 71; or a heavy chain variable region that competitively inhibits binding of an isolated antibody, antibody fragment or antigen binding fragment comprising at least one of a heavy chain variable region that includes the 3 CDRs of one of SEQ ID NO: 1, SEQ ID NO: 11, SEQ ID NO: 21, SEQ ID NO: 31, SEQ ID NO: 41, SEQ ID NO: 51, SEQ ID NO: 61, or SEQ ID NO: 71.

In other embodiments, the antibody, antibody fragment or antigen binding fragment thereof can include a heavy chain variable region that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of one of SEQ ID NO: 1, SEQ ID NO: 11, SEQ ID NO: 21, SEQ ID NO: 31, SEQ ID NO: 41, SEQ ID NO: 51, SEQ ID NO: 61, or SEQ ID NO: 71.

In still other embodiments, the antibody, antibody fragment, or antigen binding fragment thereof can include a light chain variable region that includes the 3 CDRs of one of SEQ ID NO: 6, SEQ ID NO: 16, SEQ ID NO: 26, SEQ ID NO: 36, SEQ ID NO: 46, SEQ ID NO: 56, SEQ ID NO: 66, or SEQ ID NO: 76; or a light chain variable region that competitively inhibits binding of a peptide, protein, isolated antibody, antibody fragment, or antigen binding fragment comprising at least one of a light chain variable region that includes the 3 CDRs of one of SEQ ID NO: 6, SEQ ID NO: 16, SEQ ID NO: 26, SEQ ID NO: 36, SEQ ID NO: 46, SEQ ID NO: 56, SEQ ID NO: 66, or SEQ ID NO: 76.

In some embodiments, the antibody, antibody fragment or antigen binding fragment thereof can include a light chain variable region that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of one of SEQ ID NO: 6, SEQ ID NO: 16, SEQ ID NO: 26, SEQ ID NO: 36, SEQ ID NO: 46, SEQ ID NO: 56, SEQ ID NO: 66, or SEQ ID NO: 76.

In some embodiments, the antibody, antibody fragment or antigen binding fragment thereof can include at least one of the following:
a) a heavy chain variable region that includes the 3 CDRs of SEQ ID NO: 1 and a light chain variable region that includes the 3 CDRs of SEQ ID NO: 6;
b) a heavy chain variable region that includes the 3 CDRs of SEQ ID NO: 11 and a light chain variable region that includes the 3 CDRs of SEQ ID NO: 16;
c) a heavy chain variable region that includes the 3 CDRs of SEQ ID NO: 21 and a light chain variable region that includes the 3 CDRs of SEQ ID NO: 26;
d) a heavy chain variable region that includes the 3 CDRs of SEQ ID NO: 31 and a light chain variable region that includes the 3 CDRs of SEQ ID NO: 36;
e) a heavy chain variable region that includes the 3 CDRs of SEQ ID NO: 41 and a light chain variable region that includes the 3 CDRs of SEQ ID NO: 46;
f) a heavy chain variable region that includes the 3 CDRs of SEQ ID NO: 51 and a light chain variable region that includes the 3 CDRs of SEQ ID NO: 56;
g) a heavy chain variable region that includes the 3 CDRs of SEQ ID NO: 61 and a light chain variable region that includes the 3 CDRs of SEQ ID NO: 66; or
h) a heavy chain variable region that includes the 3 CDRs of SEQ ID NO: 71 and a light chain variable region that includes the 3 CDRs of SEQ ID NO: 76.

In some embodiments, the antibody, antibody fragment or antigen binding fragment thereof can include at least one of the following:
a) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 1 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 6;
b) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 11 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 16;
c) a heavy chain variable that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 21 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 26;
d) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 31 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 36;
e) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 41 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 46;
f) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 51 and a light chain that includes an amino acid at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 56;
g) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 61 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 66; or
h) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 71 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 76.

In some embodiments, the peptide protein, antibody, antibody fragment, or antigen binding fragment thereof can include a peptide linker.

In some embodiments, the peptide, protein, antibody, antibody fragment, or antigen binding fragment thereof is humanized or chimeric.

In other embodiments, the peptide, protein, antibody, antibody fragment, or antigen binding fragment thereof binds to PTPμ, PTPκ, PCP-2, PTPμ, or PTPρ.

In other embodiment, the peptide, protein, antibody, antibody fragment, or antigen binding fragment binds to amino acids 42-60 of PTPμ, amino acids 816-914 of PTPμ, amino acids, 958-1161 of PTPμ, or amino acids 915-958 of PTPμ.

In some embodiments, the antibody, antibody fragment or antigen binding fragment has an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, or SEQ ID NO: 111.

In other embodiments, the antibody, antibody fragment or antigen binding fragment has is encoded by a nucleotide sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, or SEQ ID NO: 112.

Still other embodiments relate to a chimeric antigen receptor (CAR) comprising extracellular antigen binding domain that includes an scFv having at least one of:
a)
a CDR-H1 comprising the amino acid sequence of GFTFTDYY (SEQ ID NO: 3),
a CDR-H2 comprising the amino acid sequence of IRNKANGYRT (SEQ ID NO: 4), and
a CDR-H3 comprising the amino acid sequence of ASLSAY (SEQ ID NO: 5);
b)
a CDR-L1 comprising the amino acid sequence of KSLLHSNGITY (SEQ ID NO: 8),
a CDR-L2 comprising the amino acid sequence of QMS (SEQ ID NO: 9), and
a CDR-L3 comprising the amino acid sequence of AQNLELWT (SEQ ID NO: 10);
c)
a CDR-H1 comprising the amino acid sequence of GASITSGH (SEQ ID NO: 13),
a CDR-H2 comprising the amino acid sequence of IRDSGTT (SEQ ID NO: 14), and
a CDR-H3 comprising the amino acid sequence of VTTPFVY (SEQ ID NO: 15);
d)
a CDR-L1 comprising the amino acid sequence of QDINSY (SEQ ID NO: 18),
a CDR-L2 comprising the amino acid sequence of RAN (SEQ ID NO: 19), and
a CDR-L3 comprising the amino acid sequence of LQYDEFPLT (SEQ ID NO: 20);
e)
a CDR-H1 comprising the amino acid sequence of GFSLFNYG (SEQ ID NO: 23),
a CDR-H2 comprising the amino acid sequence of IWADGTI (SEQ ID NO: 24), and
a CDR-H3 comprising the amino acid sequence of ARHFDY (SEQ ID NO: 25);
f)
a CDR-L1 comprising the amino acid sequence of QNIVHSNGNTY (SEQ ID NO: 28),
a CDR-L2 comprising the amino acid sequence of KVS (SEQ ID NO: 29), and
a CDR-L3 comprising FQGSRLPLT (SEQ ID NO: 30);
g)
a CDR-H1 comprising the amino acid sequence of GFIFSSYY (SEQ ID NO: 33),
a CDR-H2 comprising the amino acid sequence of ININGGST (SEQ ID NO: 34), and
a CDR-H3 comprising the amino acid sequence of GRHDHGYGWFAY (SEQ ID NO: 35)
h)
a CDR-L1 comprising the amino acid sequence of QNVGRN (SEQ ID NO: 38),
a CDR-L2 comprising the amino acid sequence of SAS (SEQ ID NO: 39), and
a CDR-L3 comprising the amino acid sequence of QQYNSYPWT (SEQ ID NO: 40);
i)
a CDR-H1 comprising the amino acid sequence of GYTFTTAG (SEQ ID NO: 43),
a CDR-H2 comprising the amino acid sequence of INTQSGVS (SEQ ID NO: 44), and
a CDR-H3 comprising the amino acid sequence of ARSNSGGFPY (SEQ ID NO: 45);
j)
a CDR-L1 comprising the amino acid sequence QSLFNSTTHKIY (SEQ ID NO: 48),
a CDR-L2 comprising the amino acid sequence FAS (SEQ ID NO: 49), and
a CDR-L3 comprising the amino acid sequence of QQHYTAPWT (SEQ ID NO: 50);
k)
a CDR-H1 comprising the amino acid sequence of GYTFTTVG (SEQ ID NO: 53),
a CDR-H2 comprising the amino acid sequence of INTHSGVS (SEQ ID NO: 54), and
a CDR-H3 comprising the amino acid sequence of ARGNSNYGFPY (SEQ ID NO: 55);
l)
a CDR-L1 comprising the amino acid sequence of EDIYNR (SEQ ID NO: 58),
a CDR-L2 comprising the amino acid sequence of GAT (SEQ ID NO: 59), and
a CDR-L3 comprising the amino acid sequence of QQYWSTPWT (SEQ ID NO: 60);
m)
a CDR-H1 comprising the amino acid sequence of GYSITSDYA (SEQ ID NO: 63),
a CDR-H2 comprising the amino acid sequence of ISYRSIT (SEQ ID NO: 64), and
a CDR-H3 comprising the amino acid sequence of ARFGYDGNY (SEQ ID NO: 65);
n)
a CDR-L1 comprising the amino acid sequence of QNIVHSNGDTY (SEQ ID NO: 68),
a CDR-L2 comprising the amino acid sequence of RVS (SEQ ID NO: 69), and
a CDR-L3 comprising the amino acid sequence of FQGSHVPLT (SEQ ID NO: 70);
o)
a CDR-H1 comprising the amino acid sequence of GYTFTDYN (SEQ ID NO: 73),
a CDR-H2 comprising the amino acid sequence of INPDNGAT (SEQ ID NO: 74), and
a CDR-H3 comprising the amino acid sequence of VRGFDY (SEQ ID NO: 75);
p)
a CDR-L1 comprising the amino acid sequence of QAIVHSNGNTY (SEQ ID NO: 78),
a CDR-L2 comprising the amino acid sequence of KVS (SEQ ID NO: 79), and
a CDR-L3 comprising the amino acid sequence of FQSSHIPWT (SEQ ID NO: 80); or
a heavy chain variable region and/or light chain variable region that competitively inhibits binding of a peptide, protein, isolated antibody, antibody fragment or antigen binding fragment thereof comprising at least one of a), b), c), d), e), f), g), h), i), j), k), l), m), n), o), or p) to a CAM, such as PTPμ.

Still other embodiments relate to a chimeric antigen receptor (CAR) comprising extracellular antigen binding domain that includes a PTPμ ligand. The PTPμ ligand can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 113.

### BRIEF DESCRIPTION OF THE DRAWINGS

FIG. 1 illustrates a graph showing antibody-dependent cellular toxicity (ADCC) with a PTPμ-Fc chimeric agent.

FIG. 2 illustrates a plot showing binding of mAB and derivatives to PTPμ.

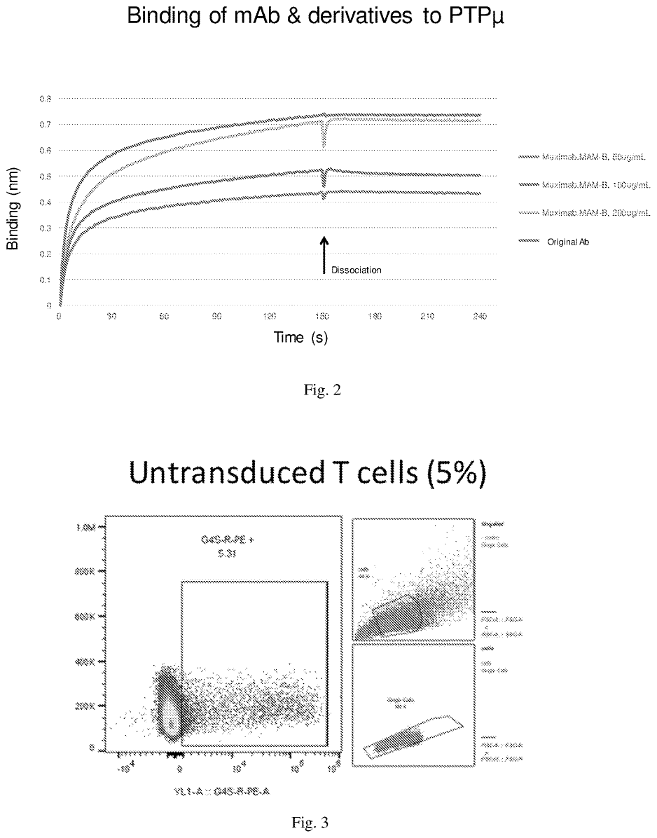

FIG. 3 illustrates images showing cells expressing the PTPμ CAR, MAM-B scFv CAR, and MAM-D scFv CAR.

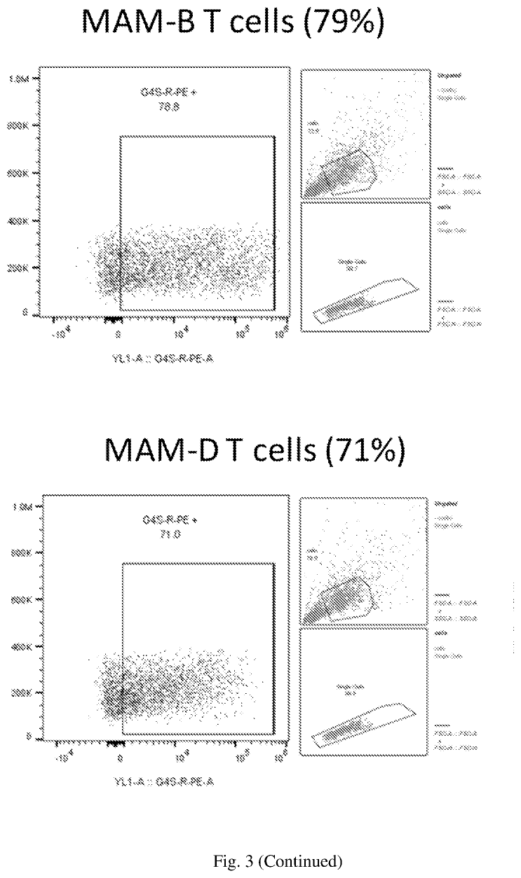

FIG. 4 illustrates images showing a first adhesion spot assay of MAM-B scFv CAR+ cells and CAR− cells.

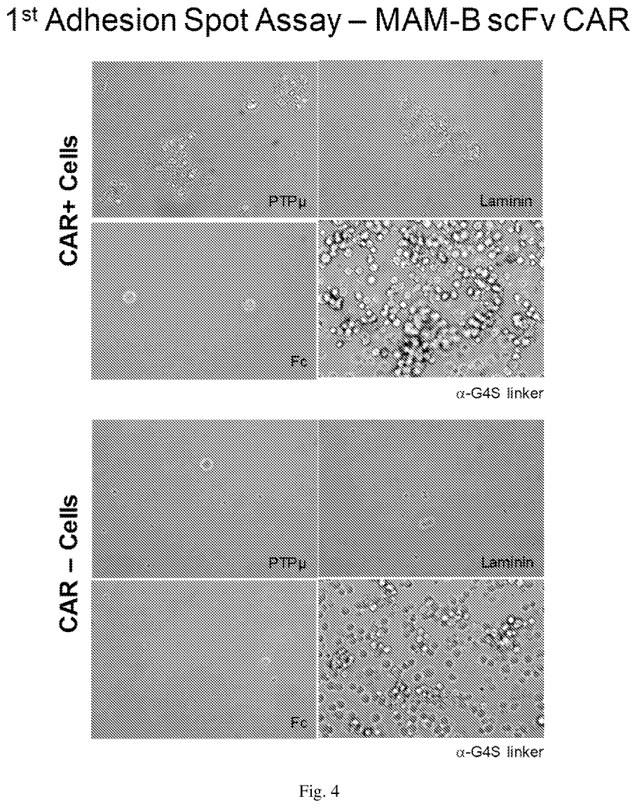

FIG. 5 illustrates images showing a second adhesion spot assay of MAM-B scFv CAR+ cells and CAR− cells.

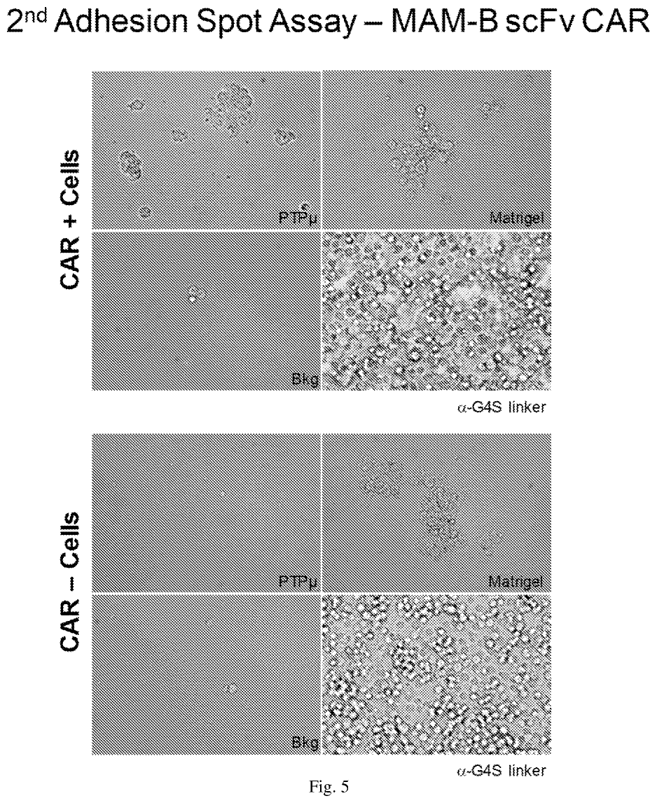

### DETAILED DESCRIPTION OF THE INVENTION

As used herein, the term “acceptor human framework” refers to a framework comprising the amino acid sequence of a VL or VH framework derived from a human immunoglobulin framework, or from a human consensus framework.

As used herein, the term “antibody” covers full length monoclonal antibodies, polyclonal antibodies, nanobodies and multi-specific antibodies. Biological antibodies are usually hetero-tetrameric glycoproteins of about 150,000 Daltons, composed of two identical light (L) chains and two identical heavy (H) chains. The two heavy chains are linked together by disulfide bonds, and each heavy chain is linked to a light chain by a disulfide bond. Each full-length IgG molecule contains at least two binding sites for a specific target or antigen. Light chains are either kappa or the lambda. Both light chains contain a domain of variable amino acid sequences, called the variable region (variously referred to as a “VL,” “Vκ,” or “Vλ-region”) and a domain of relatively conserved amino acid sequences, called the constant region (“CL-region”).

Similarly, each heavy chain contains a variable region (“VH-region”) and three constant domains (“CH1-,” “CH2-,” and “CH3-regions”) and a hinge region.

As used herein, the term “antibody fragment” refers to a segment of a full-length antibody, generally called the target binding or variable region. Other antibodies include diabodies, linear antibodies, single-chain antibody molecules and multispecific antibodies formed from antibody fragments. Examples include Fab, Fab′, F(ab′)2, Fv, or scFv fragments. An “Fv” fragment is the minimum antibody fragment which contains a complete target recognition and binding site.

As used herein, the term “antigen binding fragment” refers to a fragment or fragments of an antibody molecule that contain the antibody variable regions responsible for antigen binding.

Antigen binding fragments can be prepared from full-length antibody by protease digestion. Antigen binding fragments may be produced using standard recombinant DNA methodology by those skilled in the art. Examples of antigen binding fragments:
“Fab” fragments (monovalent fragments consisting of one constant and one variable domain of each of the heavy and light chains)
“Monovalent fragments” (antibody fragments consisting of the VL, VH, CL and CH1 domains)
“F(ab′)2” fragments (bivalent fragments comprising two Fab′ fragments linked by a disulfide bridge at the hinge region);
“Fd” fragments (which consist of the VH and CH1 domains of an antibody);
“Fv” fragment (which consist of the VL and VH domains of a single arm of an antibody); Single domain antibodies (“dAb”), which consist of a VH domain or a VL domain; and
an isolated Complementarity Determining Region (“CDR”).

As used herein, complementarity-determining region (“CDR”) are the key binding regions of the antibody. CDR refers to a specific region within variable regions of the heavy and the light chain. Generally, the variable region consists of four framework regions (FR1, FR2, FR3, FR4) and three CDRs arranged in the following manner: NH2-FR1-CDR1-FR2-CDR2-FR3-CDR3-FR4-COOH. The term “framework regions” refers to those variable domain residues other than the CDR residues herein defined.

CDRs, as antigen binding fragments, can also be incorporated into single domain antibodies, maxi bodies, mini bodies, intrabodies, diabodies, triabodies, tetra bodies, and bis-scFv. Antigen binding fragments of antibodies can be grafted into scaffolds based on polypeptides. Antigen binding fragments can be incorporated into single chain molecules comprising a pair of tandem Fd segments (VH-CH1-VH-CH1) which, together with complementary light chain polypeptides, form a pair of antigen binding regions.

As used herein, a “single chain Fv” or “scFv” antibody fragment comprises the VH and VL domains of an antibody, wherein these domains are present in a single polypeptide chain.

As used herein “effector functions” refer to those biological activities attributable to the native Fc region of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); lack of activation of platelets that express Fc receptor; and B cell activation. In order to minimize or eliminate side effects of a therapeutic antibody, it may be preferable to minimize or eliminate effector functions.

An “engineered antibody” is an antibody that is not naturally produced, and which has been altered or created to achieve a specific purpose or to have a specific characteristic. For example, antibodies which have undergone deliberate modifications to their wild type forms, to have reduced effector functions, are engineered antibodies.

As used herein, the term “Fc region” refers to the region of the antibody that provides defense to a given antigen.

As used herein, the term “first portion of the antibody” refers to a portion of a whole antibody, a portion less than the whole, which contains the antigen binding regions of the antibody. “Second portion of the antibody” refers to a portion of a whole antibody, a portion less than the whole, which consists of the portion of the antibody which is not included in the first portion.

The terms “Fc receptor” or “FcγR” describe a receptor that binds to the Fc region of an IgG. “FcγRI,” “FcγRII,” and “FcγRIII” are subclasses of FcγRs.

A “modification” to an antibody, antibody fragment, antigen binding fragment and/or Fc region of an antibody, refers to a substitution, insertion, or deletion of one or more amino acids in the protein's wild type polypeptide sequence. A modified antibody, antibody fragment, antigen binding fragment and/or Fc region is one in which a modification has been artificially made.

As used herein, “competitively inhibits” refers to competitive inhibition of binding of an isolated antibody or antigen binding portion thereof to a CAM, such as PTPμ, by any other molecule.

As used herein, the term “epitope” refers to a site on a CAM, such as PTPμ, to which antibody and fragments thereof bind and perform the functional activity. The term epitope is the same as “antigenic site”, and “antibody binding site,”. One skilled in the art can align the sequence of a CAM, such as PTPμ, of a human with the sequence of a CAM, such as PTPμ, from another animal species and determine the positions of the epitope.

As used herein, “Fab fragment” refers to the variable (VL) and constant (CL) domains of the light chain and the variable (VH) and first constant (CH1) domains of the heavy chain. Fab′ fragments differ from Fab fragments by the few extra residues at the carboxyl terminus of the heavy chain CH1 domain including one or more cysteines from the antibody hinge region. Fab′ fragments are produced by cleavage of the disulfide bond at the hinge cysteines of the F(ab′)2 pepsin digestion product.

As used herein, the term “antigen binding fragment thereof” of an antibody refers to an antibody fragment having qualitative biological activity in common with a full-length antibody. For example, an antigen binding fragment thereof of an antibody is one which can bind to a CAM, such as PTPμ.

As used herein, the term “human consensus framework” refers to a framework which represents the most commonly occurring amino acid residue in a selection of human immunoglobulin VL or VH framework sequences. Generally, the selection of human immunoglobulin VL or VH sequences is from a subgroup of variable domain sequences.

As used herein, a “humanized antibody” refers to an antibody consisting of mostly human sequences, except for CDR1, CDR2, and CDR3. All framework regions are also humanized. A chimeric antibody comprises murine CDRs, murine framework regions, and human constant regions. Collectively, chimeric antibodies contain murine both variable regions and human constant regions.

As used herein, the term “monovalent antibody or antigen binding fragment thereof” refers to an antibody or antigen binding fragment thereof comprising a single binding domain, e.g., VH or VHH, for an antigen, e.g., a single CAM, such as PTPμ molecule.

As used herein, the term “single domain antibody” defines molecules where the antigen binding site is present on, and formed by, a single immunoglobulin domain. Generally, the antigen binding site of an immunoglobulin single variable domain is formed by no more than three CDRs. The single variable domain may, for example, include a light chain variable domain sequence (a VL sequence) or a suitable fragment thereof; or a heavy chain variable domain sequence (e.g., a VH sequence or VHH sequence) or a suitable fragment thereof; as long as it is capable of forming a single antigen binding unit (i.e., a functional antigen binding unit that essentially is the single variable domain, such that the single antigen binding domain does not need to interact with another variable domain to form a functional antigen binding unit).

As used herein, the term “camelid antibody” refers to an antibody derived from a camelid species, for example, in a camel, dromedary, llama, alpaca or guanaco. Camelid antibodies differ from those of most other mammals in that they lack a light chain, and thus include only heavy chains with complete and diverse antigen binding capabilities (Hamers-Casterman, C. et al., Nature, 363:446-8, 1993).

As used herein, the term “VHH” refers to a single heavy chain variable domain antibody devoid of light chains. VHH chains, for example, can be of the type that can be found in Camelidae that are naturally devoid of light chains or to a synthetic and non-immunized VHH that can be constructed accordingly. Each heavy chain includes a variable region encoded by V-, D- and J-exons. A VHH may be a natural VHH antibody, e.g., a camelid antibody, or a recombinant protein including a heavy chain variable domain.

As used herein, the term an “isolated antibody” refers to an antibody that is substantially free of other antibodies having different antigenic specificities (e.g., an isolated antibody that binds to a CAM, such as PTPμ, is substantially free of contaminants, e.g., antibodies that do not bind to a CAM, such as PTPμ). In addition, an “isolated” antibody is one that has been identified and separated and/or recovered from a component of its natural environment. Contaminant components of its natural environment are materials that could interfere with diagnostic or therapeutic uses for the antibody, and may include enzymes, hormones, and other proteinaceous or nonproteinaceous solutes.

As used herein, the term “specific binding” of an antibody or fragment thereof, polypeptide, or peptidomimetic is binding to a target molecule that is measurably different from binding to molecules that are not target molecules. As used herein, specific binding refers to a greater than 95% preference for binding a particular antigen versus background (“non-specific”) binding. “Substantially specific” binding refers to a greater than about 80% preference for binding a particular antigen versus background. Binding can be measured using a variety of methods including, but not limited to, Western blot, immunoblot, enzyme-linked immunosorbant assay (“ELISA”), radioimmunoassay (“RIA”), immunoprecipitation, surface plasmon resonance, bio-layer interferometry, chemiluminescence, fluorescent polarization, phosphorescence, immunohistochemical analysis, matrix-assisted laser desorption/ionization time-of-flight (“MALDI-TOF”) mass spectrometry, microcytometry, microarray, microscopy, fluorescence activated cell sorting (“FACS”) and flow cytometry.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningioma, medulloblastoma, schwannoma and ependymoma.

The term “chimeric protein” or “fusion protein” is a fusion of a first amino acid sequence encoding a polypeptide with a second amino acid sequence defining a domain (e.g., polypeptide portion) foreign to and not substantially homologous with any domain of the first polypeptide. A chimeric protein may present a foreign domain, which is found (albeit in a different protein) in an organism, which also expresses the first protein, or it may be an “interspecies”, “intergenic”, etc. fusion of protein structures expressed by different kinds of organisms.

The term “epitope” includes any protein determinant capable of specific binding to an immunoglobulin or CAR. Epitope determinants usually consist of chemically active surface groupings of molecules such as amino acids or sugar side chains and usually have specific three-dimensional structural characteristics, as well as specific charge characteristics.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, melanoma, lung cancer and the like.

The term “Chimeric Antigen Receptor” or alternatively a “CAR” refers to a set of polypeptides, typically two in the simplest embodiments, which when in a T cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule. In some embodiments, the set of polypeptides are in the same polypeptide chain (e.g., comprise a chimeric fusion protein). In some embodiments, the set of polypeptides are not contiguous with each other, e.g., are in different polypeptide chains. In some embodiments, the set of polypeptides include a dimerization switch that, upon the presence of a dimerization molecule, can couple the polypeptides to one another, e.g., can couple an antigen binding domain to an intracellular signaling domain. In one embodiment, the stimulatory molecule of the CAR is the zeta chain associated with the T cell receptor complex. In one aspect, the cytoplasmic signaling domain comprises a primary signaling domain (e.g., a primary signaling domain of CD3-zeta). In one embodiment, the cytoplasmic signaling domain further comprises one or more functional signaling domains of at least one costimulatory molecule as defined below.

In one embodiment, the CAR comprises a chimeric fusion protein comprising an extracellular antigen binding domain, a transmembrane domain and an intracellular signaling domain comprising a functional signaling domain of a stimulatory molecule. In one embodiment, the CAR comprises a chimeric fusion protein comprising an extracellular antigen binding domain, a transmembrane domain and an intracellular signaling domain comprising a functional signaling domain of a co-stimulatory molecule and a functional signaling domain of a stimulatory molecule. In one embodiment, the CAR comprises a chimeric fusion protein comprising an extracellular antigen binding domain, a transmembrane domain and an intracellular signaling domain comprising two functional signaling domains of one or more co-stimulatory molecule(s) and a functional signaling domain of a stimulatory molecule. In one embodiment, the CAR comprises a chimeric fusion protein comprising an extracellular antigen binding domain, a transmembrane domain and an intracellular signaling domain comprising at least two functional signaling domains of one or more co-stimulatory molecule(s) and a functional signaling domain of a stimulatory molecule.

The terms “T lymphocyte” and “T cell” are used interchangeably and refer to a principal type of white blood cell that completes maturation in the thymus and that has various roles in the immune system, including the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupT1, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naive T cells, regulator T cells, gamma delta T cells, and the like. Additional types of helper T cells include cells such as Th3 (Treg), Th17, Th9, or Tfh cells. Additional types of memory T cells include cells such as central memory T cells (Tcm cells), effector memory T cells (Tem cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

“CD4+ T cells” refers to a subset of T cells that express CD4 on their surface and are associated with cell-mediated immune response. They are characterized by the secretion profiles following stimulation, which may include secretion of cytokines such as IFN-γ, TNF-α, IL2, IL4 and IL10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8+ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

The term “NK cell” or “Natural Killer cell” refer to a subset of peripheral blood lymphocytes defined by the expression of CD56 or CD16 and the absence of the T cell receptor (CD3). As used herein, the terms “adaptive NK cell” and “memory NK cell” are interchangeable and refer to a subset of NK cells that are phenotypically CD3− and CD56+, expressing at least one of NKG2C and CD57, and optionally, CD16, but lack expression of one or more of the following: PLZF, SYK, FcRγ, and EAT-2. In some embodiments, isolated subpopulations of CD56+ NK cells comprise expression of CD16, NKG2C, CD57, NKG2D, NCR ligands, NKp30, NKp40, NKp46, activating and inhibitory KIRs, NKG2A and/or DNAM-1. CD56+ can be dim or bright expression.

The term “NKT cells” or “natural killer T cells” refers to CD1d-restricted T cells, which express a T cell receptor (TCR). Unlike conventional T cells that detect peptide antigens presented by conventional major histocompatibility (MHC) molecules, NKT cells recognize lipid antigens presented by CD1d, a non-classical MHC molecule. Two types of NKT cells are recognized. Invariant or type I NKT cells express a very limited TCR repertoire—a canonical α-chain (Vα24-Jα18 in humans) associated with a limited spectrum of β chains (β11 in humans). The second population of NKT cells, called non-classical or non-invariant type II NKT cells, display a more heterogeneous TCRαβ usage. Type I NKT cells are considered suitable for immunotherapy. Adaptive or invariant (type I) NKT cells can be identified with the expression of at least one or more of the following markers, TCR Va24-Ja18, Vb11, CD1d, CD3, CD4, CD8, aGalCer, CD161 and CD56.

As used herein, the term “identical” or “substantially identical” with respect to an antibody chain polypeptide sequence may be construed as an antibody chain exhibiting at least 65%, 70%, 80%, 90% or 95% sequence identity to the reference polypeptide sequence present in the variable region of the antigen binding fragment. The term with respect to a nucleic acid sequence may be construed as a sequence of nucleotides exhibiting at least about 65%, 75%, 85%, 90%, 95% or 97% sequence identity to the reference nucleic acid sequence.

As used herein, the term “individual” refers to a vertebrate, preferably a mammal and more preferably a human. Individuals amenable to treatment include those who are presently asymptomatic, but who are at risk of developing a symptomatic disorder in which the alternative complement pathway plays a role, or in which activation of the alternative complement pathway plays a role.

As used herein, the term “mammal” refers to any animal classified as a mammal includes humans, higher primates, domestic and farm animals, horses, pigs, cattle, dogs, cats and ferrets, etc. In one embodiment of the invention, the mammal is a human.

As used herein, “monoclonal antibody” refers to a homogeneous population of antibodies. Such antibodies are highly specific and are directed against a single target antigen. These monoclonal antibodies are homogeneously produced by the hybridoma culture, uncontaminated by other immunoglobulins. Monoclonal antibodies can also be produced by other procedures such as phase display by well known methods.

As used herein, the term “treatment” refers to both therapeutic treatment and prophylactic or preventative measures.

The term “gene” or “recombinant gene” refers to a nucleic acid comprising an open reading frame encoding a polypeptide, including both exon and (optionally) intron sequences.

The term “homology” and “identity” are used synonymously throughout and refer to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous or identical at that position. A degree of homology or identity between sequences is a function of the number of matching or homologous positions shared by the sequences.

The term “mutant” refers to any change in the genetic material of an organism, in particular a change (i.e., deletion, substitution, addition, or alteration) in a wild type polynucleotide sequence or any change in a wild type protein. The term “variant” is used interchangeably with “mutant”. Although it is often assumed that a change in the genetic material results in a change of the function of the protein, the terms “mutant” and “variant” refer to a change in the sequence of a wild type protein regardless of whether that change alters the function of the protein (e.g., increases, decreases, imparts a new function), or whether that change has no effect on the function of the protein (e.g., the mutation or variation is silent).

The term “nucleic acid” refers to polynucleotides, such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, and intraspinal injection and infusion.

The phrases “systemic administration,” “administered systemically,” “peripheral administration” and “administered peripherally” as used herein mean the administration of a compound, agent or other material other than directly into a specific tissue, organ, or region of the subject being treated (e.g., brain), such that it enters the animal's system and, thus, is subject to metabolism and other like processes, for example, subcutaneous administration.

The terms “patient”, “subject”, “mammalian host,” and the like are used interchangeably herein, and refer to mammals, including human and veterinary subjects.

The terms “peptide(s)”, “protein(s)” and “polypeptide(s)” are used interchangeably herein. As used herein, “polypeptide” refers to any peptide or protein comprising two or more amino acids joined to each other by peptide bonds or modified peptide bonds (i.e., peptide isomers). “Polypeptide(s)” refers to both short chains, commonly referred as peptides, oligopeptides or oligomers, and to longer chains generally referred to as proteins.

The terms “polynucleotide sequence” and “nucleotide sequence” are also used interchangeably herein.

“Recombinant,” as used herein, means that a protein is derived from a prokaryotic or eukaryotic expression system.

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

The phrase “therapeutically effective amount” or “pharmaceutically effective amount” is an art-recognized term. In certain embodiments, the term refers to an amount of a therapeutic agent that produces some desired effect at a reasonable benefit/risk ratio applicable to any medical treatment. In certain embodiments, the term refers to that amount necessary or sufficient to eliminate, reduce or maintain a target of a particular therapeutic regimen. The effective amount may vary depending on such factors as the disease or condition being treated, the particular targeted constructs being administered, the size of the subject or the severity of the disease or condition. One of ordinary skill in the art may empirically determine the effective amount of a particular compound without necessitating undue experimentation. In certain embodiments, a therapeutically effective amount of a therapeutic agent for in vivo use will likely depend on a number of factors, including: the rate of release of an agent from a polymer matrix, which will depend in part on the chemical and physical characteristics of the polymer; the identity of the agent; the mode and method of administration; and any other materials incorporated in the polymer matrix in addition to the agent.

The term “wild type” refers to the naturally-occurring polynucleotide sequence encoding a protein, or a portion thereof, or protein sequence, or portion thereof, respectively, as it normally exists in vivo.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to cell adhesion molecule (CAM) targeted immunotherapies and, particularly, peptides, proteins, antibodies, antibody fragments, and antigen-binding fragments thereof that bind to CAMs and their use in immunotherapies. Proteolysis of CAMs is a common event in cancer and cleaved fragments of CAMs can function as tumor biomarkers. Full length CAMs including the Ig superfamily receptor protein tyrosine phosphatase, PTPμ, function as tumor suppressors and normally contribute to cell-cell adhesion by binding homophilically to PTPμ on adjacent cells to link cells together. In the microenvironment of multiple tumor types, PTPμ proteolysis creates an extracellular domain (ECD) fragment that leads to the formation of unique epitopes (neoantigens) expressed exclusively on the surface of tumor cells.

We developed immunotherapies based on peptides, proteins, antibodies, antibody fragments thereof, and antigen binding fragments thereof that bind to CAMs, such as PTPμ. The peptides, proteins, antibodies, antibody fragments, and antigen binding fragments thereof can include peptides and proteins that bind to CAMs, such as PTPμ, humanized and chimeric versions of antibodies, antibody fragments thereof, and antigen binding fragments that bind to CAMs, such as PTPμ, scFv antibody fragments that bind to CAMs, such as PTPμ, as well as Fc domain chimeric proteins, in which the Fc domain of the human immunoglobulin heavy chain IgG1 is conjugated to a tumor specific protein or a ligand of that protein that binds to CAMs, such as PTPμ.

The Fc domain binds to Fcγ receptors (FcγR) on innate immune cells triggering antibody-dependent cellular cytotoxicity (ADCC) by natural killer (NK) cells and phagocytosis of tumor cells by macrophages in the periphery or microglia in the brain. Additionally, the FcγR on dendritic cells (DC) present tumor antigens to T cells.

In some embodiments, the peptide, protein, antibody, antibody fragment, or antigen binding fragment thereof can include a PTPμ-Fc chimeric agent that stimulates the immune system to target and kill tumor cells. By combining the tumor targeting potential of PTPμ with the body's own immune recognition, the PTPμ-Fc chimeric agent can trigger ADCC.

The peptides, proteins, antibodies, antibody fragments, and antigen binding fragments thereof can include humanized and chimeric antibodies, antibody fragments or antigen binding fragments thereof that selectively bind to a CAM, such as the proteolytically cleaved extracellular fragment of PTPμ. The antibodies, antibody fragments and antigen binding fragments thereof can be used in an immunotherapy to treat cancer in humans or other mammals.

Methods for making humanized antibodies from non-human versions are well known in the art. Humanization is essentially performed by substituting rodent CDRs or CDR sequences for the corresponding sequences of a human antibody. The choice of human variable domains, both light and heavy, to be used in making the humanized antibodies can, in some instances, be important to reduce antigenicity and/or human anti-mouse antibody (HAMA) response.

The variable domain of the antibodies refers to certain portions of the variable domains that differ in sequence among antibodies. The variability in the antibodies and antigen binding fragments thereof can be concentrated in three CDR segments, located in both the light chain and the heavy chain variable domains. The highly conserved portions of variable domains are called framework (FR) regions. In the antibodies described herein, there are four FR regions, connected by three CDRs, that can comprise a variable chain. The CDRs in each of the light and heavy chains are held together in close proximity by the FR regions and, with the CDRs from the other chain, can contribute to the formation of the target binding site of antibodies.

Antibody humanization is a process that can generate engineered human antibodies with variable region (“V-region”) sequences that are substantially similar to actual human germline sequences, while retaining the binding specificity and affinity of a reference antibody. This process can graft, for example, the CDR1, CDR2, and CDR3 regions of the heavy and the light chain sequences into humanized human framework that is both optimized and previously identified prior to the start of the grafting process. The variable region containing the humanized framework can be produced into Fab, Fab′, F(ab′)2, or single chain antigen binding fragments thereof. The resulting engineered humanized antibody fragments can retain the binding specificity of the parent murine antibody for the CAM, and can have an equivalent or higher binding affinity for a specific antigen than the parent antibody. The engineered antigen binding fragments can have heavy and light chain V-regions with a high degree of amino acid sequence identity compared to the closest human germline antibody genes. For example, additional maturational changes can be introduced in the CDR3 regions of each chain during construction in order to identify antibodies with optimal binding kinetics.

In some embodiments, the antibody may be a single-domain antibody, such as a VHH or V-NAR. Such antibodies exist naturally in camelids and sharks (Saerens, D. et al., Curr. Opin. Pharmacol., 8:600-8, 2008). Camelid antibodies are described in, for example. U.S. Pat. Nos. 5,759,808; 5,800,988; 5,840,526; 5,874,541; 6,005,079; and 6,015,695, the entire contents of each of which are incorporated herein by reference. The cloned and isolated VHH domain is a stable polypeptide that features the full antigen-binding capacity of the original heavy-chain antibody. VHH domains, with their unique structural and functional properties, combine the advantages of conventional antibodies (high target specificity, high target affinity and low inherent toxicity) with important features of small molecule drugs (the ability to inhibit enzymes and access receptor clefts). Furthermore, they are stable, have the potential to be administered by means other than injection, are easier to manufacture.

In some embodiments, the humanized and chimeric antibodies, antibody fragments, and antigen binding fragments thereof can bind to the same epitope on CAM as the antibodies recited in this application. Such humanized and chimeric antibodies, antibody fragments, and antigen binding fragments thereof can be identified based on their ability to cross-compete with or competitively inhibit the antibodies and antigen binding fragments thereof in standard CAM binding assays. Thus, all humanized and chimeric antibodies, antibody fragments, and antigen binding fragments thereof that competitively inhibit the binding of antibodies and the antigen binding fragments thereof are encompassed by this disclosure.

The antigen binding fragments of antibodies can be identified following protease digestion. These include, for example, the “Fab fragment”, “Fab′ fragment” (a Fab with the heavy chain hinge region), and “F(ab′)2 fragment” (a dimer of Fab′ fragments joined at the heavy chain hinge region). Recombinant methods have been used to generate such fragments and to generate even smaller antibody fragments, e.g., those referred to as “single chain Fv” (variable fragment) or “scFv,” consisting of VL and VH joined by a synthetic peptide linker (VL-linker-VH or VH-linker-VL). Fab fragments, Fab′ fragments and scFv fragments are monovalent or monospecific for antigen binding, as they each include only one antigen binding domain including one VH/VL dimer.

Even smaller monovalent antibody fragments are the dAbs, which include only a single immunoglobulin variable domain, e.g., VH or VL, that alone specifically binds antigen, i.e., without the need for a complementary VL or VH domain, respectively. A dAb binds antigen independently of other V domains; however, a dAb can be present in a homo- or hetero-multimer with other VH or VL domains where the other domains are not required for antigen binding by the dAb, i.e., where the dAb binds antigen independently of the additional VH or VL domains.

In some embodiments, an antibody, antibody fragment or antigen binding fragment thereof can include at least one of:
a)
a CDR-H1 comprising the amino acid sequence of GFTFTDYY (SEQ ID NO: 3),
a CDR-H2 comprising the amino acid sequence of IRNKANGYRT (SEQ ID NO: 4), and
a CDR-H3 comprising the amino acid sequence of ASLSAY (SEQ ID NO: 5);
b)
a CDR-L1 comprising the amino acid sequence of KSLLHSNGITY (SEQ ID NO: 8),
a CDR-L2 comprising the amino acid sequence of QMS (SEQ ID NO: 9), and
a CDR-L3 comprising the amino acid sequence of AQNLELWT (SEQ ID NO: 10);
c)
a CDR-H1 comprising the amino acid sequence of GASITSGH (SEQ ID NO: 13),
a CDR-H2 comprising the amino acid sequence of IRDSGTT (SEQ ID NO: 14), and
a CDR-H3 comprising the amino acid sequence of VTTPFVY (SEQ ID NO: 15);
d)
a CDR-L1 comprising the amino acid sequence of QDINSY (SEQ ID NO: 18),
a CDR-L2 comprising the amino acid sequence of RAN (SEQ ID NO: 19), and
a CDR-L3 comprising the amino acid sequence of LQYDEFPLT (SEQ ID NO: 20);
e)
a CDR-H1 comprising the amino acid sequence of GFSLFNYG (SEQ ID NO: 23),
a CDR-H2 comprising the amino acid sequence of IWADGTI (SEQ ID NO: 24), and
a CDR-H3 comprising the amino acid sequence of ARHFDY (SEQ ID NO: 25);
f)
a CDR-L1 comprising the amino acid sequence of QNIVHSNGNTY (SEQ ID NO: 28),
a CDR-L2 comprising the amino acid sequence of KVS (SEQ ID NO: 29), and
a CDR-L3 comprising FQGSRLPLT (SEQ ID NO: 30);
g)
a CDR-H1 comprising the amino acid sequence of GFIFSSYY (SEQ ID NO: 33),
a CDR-H2 comprising the amino acid sequence of ININGGST (SEQ ID NO: 34), and
a CDR-H3 comprising the amino acid sequence of GRHDHGYGWFAY (SEQ ID NO: 35)
h)
a CDR-L1 comprising the amino acid sequence of QNVGRN (SEQ ID NO: 38),
a CDR-L2 comprising the amino acid sequence of SAS (SEQ ID NO: 39), and
a CDR-L3 comprising the amino acid sequence of QQYNSYPWT (SEQ ID NO: 40);
i)
a CDR-H1 comprising the amino acid sequence of GYTFTTAG (SEQ ID NO: 43),
a CDR-H2 comprising the amino acid sequence of INTQSGVS (SEQ ID NO: 44), and
a CDR-H3 comprising the amino acid sequence of ARSNSGGFPY (SEQ ID NO: 45);
j)
a CDR-L1 comprising the amino acid sequence QSLFNSTTHKIY (SEQ ID NO: 48),
a CDR-L2 comprising the amino acid sequence FAS (SEQ ID NO: 49), and
a CDR-L3 comprising the amino acid sequence of QQHYTAPWT (SEQ ID NO: 50);
k)
a CDR-H1 comprising the amino acid sequence of GYTFTTVG (SEQ ID NO: 53),
a CDR-H2 comprising the amino acid sequence of INTHSGVS (SEQ ID NO: 54), and
a CDR-H3 comprising the amino acid sequence of ARGNSNYGFPY (SEQ ID NO: 55);
l)
a CDR-L1 comprising the amino acid sequence of EDIYNR (SEQ ID NO: 58),
a CDR-L2 comprising the amino acid sequence of GAT (SEQ ID NO: 59), and
a CDR-L3 comprising the amino acid sequence of QQYWSTPWT (SEQ ID NO: 60);
m)
a CDR-H1 comprising the amino acid sequence of GYSITSDYA (SEQ ID NO: 63),
a CDR-H2 comprising the amino acid sequence of ISYRSIT (SEQ ID NO: 64), and
a CDR-H3 comprising the amino acid sequence of ARFGYDGNY (SEQ ID NO: 65);
n)
a CDR-L1 comprising the amino acid sequence of QNIVHSNGDTY (SEQ ID NO: 68),
a CDR-L2 comprising the amino acid sequence of RVS (SEQ ID NO: 69), and
a CDR-L3 comprising the amino acid sequence of FQGSHVPLT (SEQ ID NO: 70);
o)
a CDR-H1 comprising the amino acid sequence of GYTFTDYN (SEQ ID NO: 73),
a CDR-H2 comprising the amino acid sequence of INPDNGAT (SEQ ID NO: 74), and
a CDR-H3 comprising the amino acid sequence of VRGFDY (SEQ ID NO: 75);
p)
a CDR-L1 comprising the amino acid sequence of QAIVHSNGNTY (SEQ ID NO: 78),
a CDR-L2 comprising the amino acid sequence of KVS (SEQ ID NO: 79), and
a CDR-L3 comprising the amino acid sequence of FQSSHIPWT (SEQ ID NO: 80); or
a heavy chain variable region and/or light chain variable region that competitively inhibits binding of a peptide, protein, isolated antibody, antibody fragment or antigen binding fragment thereof comprising at least one of a), b), c), d), e), f), g), h), i), j), k), l), m), n), o), or p) to a CAM, such as PTPμ.

In some embodiments, the antibody, antibody fragment or antigen binding fragment thereof can include a heavy chain variable region that includes the 3 CDRs of one of SEQ ID NO: 1, SEQ ID NO: 11, SEQ ID NO: 21, SEQ ID NO: 31, SEQ ID NO: 41, SEQ ID NO: 51, SEQ ID NO: 61, or SEQ ID NO: 71; or a heavy chain variable region that competitively inhibits binding of an antibody or antigen binding fragment comprising at least one of a heavy chain variable region that includes the 3 CDRs of one of SEQ ID NO: 1, SEQ ID NO: 11, SEQ ID NO: 21, SEQ ID NO: 31, SEQ ID NO: 41, SEQ ID NO: 51, SEQ ID NO: 61, or SEQ ID NO: 71.

In other embodiments, the antibody, antibody fragment or antigen binding fragment thereof can include a heavy chain variable region that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of one of SEQ ID NO: 1, SEQ ID NO: 11, SEQ ID NO: 21, SEQ ID NO: 31, SEQ ID NO: 41, SEQ ID NO: 51, SEQ ID NO: 61, or SEQ ID NO: 71.

In still other embodiments, the antibody, antibody fragment or antigen binding fragment thereof can include a light chain variable region that includes the 3 CDRs of one of SEQ ID NO: 6, SEQ ID NO: 16, SEQ ID NO: 26, SEQ ID NO: 36, SEQ ID NO: 46, SEQ ID NO: 56, SEQ ID NO: 66, or SEQ ID NO: 76; or a light chain variable region that competitively inhibits binding of a peptide, protein, isolated antibody, antibody fragment or antigen binding fragment comprising at least one of a light chain variable region that includes the 3 CDRs of one of SEQ ID NO: 6, SEQ ID NO: 16, SEQ ID NO: 26, SEQ ID NO: 36, SEQ ID NO: 46, SEQ ID NO: 56, SEQ ID NO: 66, or SEQ ID NO: 76.

In some embodiments, the antibody, antibody fragment or antigen binding fragment thereof can include a light chain variable region that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of one of SEQ ID NO: 6, SEQ ID NO: 16, SEQ ID NO: 26, SEQ ID NO: 36, SEQ ID NO: 46, SEQ ID NO: 56, SEQ ID NO: 66, or SEQ ID NO: 76.

In some embodiments, the antibody, antibody fragment or antigen binding fragment thereof can include at least one of the following:
a) a heavy chain variable region that includes the 3 CDRs of SEQ ID NO: 1 and a light chain variable region that includes the 3 CDRs of SEQ ID NO: 6;
b) a heavy chain variable region that includes the 3 CDRs of SEQ ID NO: 11 and a light chain variable region that includes the 3 CDRs of SEQ ID NO: 16;
c) a heavy chain variable region that includes the 3 CDRs of SEQ ID NO: 21 and a light chain variable region that includes the 3 CDRs of SEQ ID NO: 26;
d) a heavy chain variable region that includes the 3 CDRs of SEQ ID NO: 31 and a light chain variable region that includes the 3 CDRs of SEQ ID NO: 36;
e) a heavy chain variable region that includes the 3 CDRs of SEQ ID NO: 41 and a light chain variable region that includes the 3 CDRs of SEQ ID NO: 46;
f) a heavy chain variable region that includes the 3 CDRs of SEQ ID NO: 51 and a light chain variable region that includes the 3 CDRs of SEQ ID NO: 56;
g) a heavy chain variable region that includes the 3 CDRs of SEQ ID NO: 61 and a light chain variable region that includes the 3 CDRs of SEQ ID NO: 66; or
h) a heavy chain variable region that includes the 3 CDRs of SEQ ID NO: 71 and a light chain variable region that includes the 3 CDRs of SEQ ID NO: 76.

In some embodiments, the antibody, antibody fragment or antigen binding fragment thereof can include at least one of the following:

In some embodiments, the antibody, antibody fragment or antigen binding fragment thereof can include at least one of the following:
a) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 1 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 6;
b) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 11 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 16;
c) a heavy chain variable that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 21 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 26;
d) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 31 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 36;
e) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 41 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 46;
f) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 51 and a light chain that includes an amino acid at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 56;
i) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 61 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 66; or
j) a heavy chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 71 and a light chain that includes an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the amino acid sequence of SEQ ID NO: 76.

In some embodiments, the peptide, protein, antibody, antibody fragment, or antigen binding fragment thereof is humanized or chimeric.

In other embodiments, the peptide, protein, antibody, antibody fragment or antigen binding fragment thereof binds to PTPμ, PTPκ, PCP-2, PTPμ, or PTPρ.

In other embodiment, the peptide, protein, antibody, antibody fragment, or antigen binding fragment binds to amino acids 42-60 of PTPμ, amino acids 816-914 of PTPμ, amino acids, 958-1161 of PTPμ, or amino acids 915-958 of PTPμ.

In some embodiments, the antibody, antibody fragment or antigen binding fragment has an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, or SEQ ID NO: 111.

In other embodiments, the antibody, antibody fragment, or antigen binding fragment has is encoded by a nucleotide sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, or SEQ ID NO: 112.

In some embodiments, the peptide, protein, antibody, antibody fragment, or antigen binding fragment thereof can include a linker. The linker can be used as a linkage or connection between polypeptides or protein domains and/or associated non-protein moieties of the peptide, protein, antibody, antibody fragment or antigen binding fragment thereof. In some embodiments, a linker is a linkage or connection between at least two polypeptide constructs, e.g., such that the two polypeptide constructs are joined to each other in tandem series (e.g., an antibody or antigen binding fragment thereof linked to a second polypeptide or antibody). A linker can attach the N-terminus or C-terminus of one antibody construct to the N-terminus or C-terminus of a second polypeptide construct.

A linker can be a simple covalent bond, e.g., a peptide bond, a synthetic polymer, e.g., a polyethylene glycol (PEG) polymer, or any kind of bond created from a chemical reaction, e.g., chemical conjugation. In the case that a linker is a peptide bond, the carboxylic acid group at the C-terminus of one protein domain can react with the amino group at the N-terminus of another protein domain in a condensation reaction to form a peptide bond. Specifically, the peptide bond can be formed from synthetic means through a conventional organic chemistry reaction well-known in the art, or by natural production from a host cell, wherein a polynucleotide sequence encoding the DNA sequences of both proteins, e.g., two antibody constructs, in tandem series can be directly transcribed and translated into a contiguous polypeptide encoding both proteins by the necessary molecular machineries, e.g., DNA and RNA polymerase and ribosomes, in the host cell.

In the case that a linker is a synthetic polymer, e.g., a PEG polymer, the polymer can be functionalized with reactive chemical functional groups at each end to react with the terminal amino acids at the connecting ends of two proteins.

In the case that a linker (except peptide bond mentioned above) is made from a chemical reaction, chemical functional groups, e.g., amine, carboxylic acid, ester, azide, or other functional groups commonly used in the art, can be attached synthetically to the C-terminus of one protein and the N-terminus of another protein, respectively. The two functional groups can then react to through synthetic chemistry means to form a chemical bond, thus connecting the two proteins together. Such chemical conjugation procedures are routine for those skilled in the art.

A linker between two peptide constructs can be an amino acid linker including from 1-200 (e.g., 1-4, 1-10, 1-20, 1-30, 1-40, 2-10, 2-12, 2-16, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200) amino acids. Suitable peptide linkers are known in the art, and include, for example, peptide linkers containing flexible amino acid residues such as glycine and serine.

In some embodiments, the antibody, antibody fragment or antigen binding fragment thereof use a single domain antibody that is a heavy chain variable domain (VH, e.g., VHH) or a light chain domain (VL). Thus, one means of generating single domain antibodies specific for CAM is to amplify and express the VH and VL regions of the heavy chain and light chain gene sequences isolated, for example, from a hybridoma (e.g., a mouse hybridoma) that expresses CAM monoclonal antibody. The boundaries of VH and VL domains are set out, for example, by Kabat et al. (Sequences of Proteins of Immunological Interest (National Institutes of Health, Bethesda, Md., 1991). The information regarding the boundaries of the VH and VL domains of heavy and light chain genes is used to design PCR primers that amplify the V domain from a heavy or light chain coding sequence encoding an antibody known to bind a CAM, such as PTPμ.

The amplified V domains are inserted into a suitable expression vector, e.g., pHEN-1 (Hoogenboom, H. et al., Nucleic Acids Res., 19:4133-7, 1991) and expressed, for example, as a fusion of the VH and VL in a scFv or other suitable monovalent format. The resulting polypeptide can then be screened for high affinity monovalent binding to a CAM, such as PTPμ. Screening for binding can be performed by methods known in the art. Single domain antibodies can be generated using methods known in the art (WO2005118642; Ward, E. et al., Nature, 341:544-6, 1989; Holt, L. et al., Trends Biotechnol., 21:484-90, 2003). Each light chain domain may be either of the kappa or lambda subgroup. Methods for isolating VH and VL domains have been described in the art (EP0368684).

In one embodiment, the antibody, antibody fragment, or antigen binding fragment thereof can include a single domain antibody that is obtained from a human, humanized rodent, camelid or shark. Any such single domain antibody can be optionally humanized. Humanization of camelid single domain antibodies requires the introduction and mutagenesis of a limited number of amino acids in a single polypeptide chain. This is in contrast to humanization of scFv, Fab, (Fab′)2 and IgG, which requires the introduction of amino acid changes in two chains, the light and the heavy chain and the preservation of the assembly of both chains.

In some embodiments, the single domain antibody includes VHH domains. In some embodiments, the VHH domains correspond to the VHH domains of naturally occurring heavy chain antibodies directed against a CAM, such as PTPμ. Such VHH sequences can be generated, for example, by suitably immunizing a species of camelid with a CAM, such as PTPmu, (i.e., so as to raise an immune response and/or heavy chain antibodies directed against a CAM, such as PTPμ) by obtaining a suitable biological sample from said camelid (such as a blood sample, serum sample or sample of B-cells), and by generating VHH sequences directed against a CAM, such as PTPμ, starting from said sample, using any suitable technique known in the art (e.g., the gene encoding the single domain antibody may be cloned by PCR, or the B-cell(s) encoding the single domain antibody may be immortalized by EBV transformation, or by fusion to an immortal cell line).

Alternatively, such naturally occurring VHH domains against CAMs, such a PTPμ, can be obtained from naive libraries of camelid VHH sequences, for example by screening such a library using a CAM, such as PTPμ, or at least one part, fragment, antigenic determinant or epitope thereof using one or more screening techniques known in the art (WO 99/37681, WO 01/90190, WO 03/025020 and WO 03/035694). Alternatively, improved synthetic or semi-synthetic libraries derived from naive VHH libraries may be used, such as VHH libraries obtained from naive VHH libraries by techniques such as random mutagenesis and/or CDR shuffling (WO 00/43507). In a certain embodiment, a VHH library is constructed, transformed into a host cell, and expressed on phages after infection of the host cell with a helper phage. After several rounds of bio-panning, single domain antibodies against human CAM, such as PTPμ, can be isolated and efficiently expressed.

A library of fusion proteins including VHH or VHH fragments can be displayed on a phage, or suitable microorganism (such as yeast), to facilitate screening.

Suitable methods, techniques and host organisms for displaying and screening (a set, collection or library of) fusion proteins including VHH or VHH fragments are known in the art (WO 03/054016; Hoogenboom, H., Nat. Biotechnol., 23:1105-16, 2005).

In an additional embodiment, a method for generating fusion proteins including VHH or VHH fragment sequences includes at least the steps of: a) providing a collection or sample of cells derived from a species of camelid that express immunoglobulin sequences; b) screening the collection or sample of cells for (i) cells that express an immunoglobulin sequence that can bind to and/or have affinity for a CAM, such as PTPμ; and (ii) cells that express heavy chain antibodies, in which substeps (i) and (ii) can be performed essentially as a single screening step or in any suitable order as two separate screening steps, to provide at least one cell that expresses a heavy chain antibody that can bind to and/or has affinity for a CAM, such as PTPμ; and c) isolating from the cell the VHH sequence present in the heavy chain antibody that can bind to and/or has affinity for a CAM, such as PTPμ, followed by expressing the VHH domain.

The method for generating an amino acid sequence directed against CAM can include at least the steps of: a) providing a set, collection or library of nucleic acid sequences encoding heavy chain antibodies or VHH sequences; b) screening the set, collection or library of nucleic acid sequences for nucleic acid sequences that encode a heavy chain antibody or a fusion protein including the VHH sequence that can bind to and/or has affinity for CAM; and c) isolating the nucleic acid sequence, followed by expressing the VHH sequence present in the heavy chain antibody or by expressing the fusion protein including the VHH sequence, respectively.

Other suitable methods and techniques for obtaining the single domain antibodies and/or nucleic acids encoding the same, starting from naturally occurring VH sequences or VHH sequences may, for example, include combining one or more parts of one or more naturally occurring VH sequences (such as one or more framework region (FR) sequences and/or CDR sequences), one or more parts of one or more naturally occurring VHH sequences (such as one or more framework region sequences or CDR sequences), and/or one or more synthetic or semi-synthetic sequences, in a suitable manner, so as to provide a monovalent single domain antibody or a nucleotide sequence or nucleic acid encoding the same. Nucleotide sequences encoding framework sequences of VHH or single domain antibodies are known in the art and may alternatively be obtained by polymerase chain reaction (PCR) starting from the nucleotide sequences obtained using the methods described herein. Such compositions can be suitably combined with nucleotide sequences that encode the desired CDRs (for example, by PCR assembly using overlapping primers), to provide a single domain antibody, or antibody fragment fused with a regulator of the alternative complement pathway or fragment thereof.

Antibody or antigen binding fragments that recognize the same epitope as a parent antibody can be generated by known techniques. For example, antibody or antigen binding fragments can be prepared by proteolytic hydrolysis of an antibody or by expression in E. coli of the DNA coding for the fragment. The antibody or antigen binding fragments are antigen binding portions of an antibody, such as Fab, F(ab′)2, and scFV, and can be obtained by pepsin or papain digestion of whole antibodies by conventional methods or by genetic engineering techniques.

An antibody or antigen binding fragment can be produced by enzymatic cleavage of antibodies with pepsin to provide a 100 kDa fragment denoted F(ab′)2. This fragment can be further cleaved using a thiol reducing agent, and optionally a blocking group for the sulfhydryl groups resulting from cleavage of disulfide linkages, to produce 50 kDa Fab′ monovalent fragments. Alternatively, an enzymatic cleavage using papain produces two monovalent Fab fragments and an Fc fragment directly (U.S. Pat. Nos. 4,036,945 and 4,331,647; Nisonoff, A. et al., Arch. Biochem. Biophys., 89:230-44, 1960; Porter, R., Biochem. J., 73:119-26, 1959; Edelman et al., in Methods in Enzymology Vol. I, page 422 (Academic Press 1967), and Coligan et al., Current Protocols in Immunology, Vol. 1, pages 2.8.1-2.8.10 and 2.10.-2.10.4 (John Wiley & Sons 1991).

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

Another form of an antibody or antigen binding fragment is a peptide coding for a single complementarity-determining region (CDR). CDR peptides can be obtained by constructing genes encoding the CDR of an antibody of interest. Such genes are prepared, for example, by using reverse transcriptase followed by the polymerase chain reaction to synthesize the variable region from RNA of antibody producing cells (Larrick, J & Fry, K. METHODS—a companion to Methods in Enzymology Volume: New Techniques in Antibody Generation, 2:106-110, 1991); Courtenay-Luck, “Genetic Manipulation of Monoclonal Antibodies,” in Monoclonal Antibodies: Production, Engineering and Clinical Application, Ritter et al. (eds.), pages 166-179 (Cambridge University Press 1995); and Ward et al., “Genetic Manipulation and Expression of Antibodies,” in Monoclonal Antibodies: Principles And Applications, Birch et al., (eds.), pages 137-185 (Wiley-Liss, Inc. 1995)).

Other antibody or antigen binding fragments, for example single domain antibody fragments, are known in the art and may be used in the claimed constructs (Muyldermans, S. et al., Trends Biochem. Sci., 26:230-5, 2001; Yau, K. et al., J. Immunol. Methods, 281:161-75, 2003; Maass, D. et al., J. Immunol. Methods, 324:13-25, 2007). The VHH may have potent antigen binding capacity and can interact with novel epitopes that are inaccessible to conventional VH-VL pairs. Camelidae may be immunized with known antigens, such as a CAM, such as PTPμ, and VHHs can be isolated that bind to and neutralize the target antigen.

Still other embodiments relate to a chimeric antigen receptor (CAR) comprising extracellular antigen binding domain that includes antigen binding domains of an antibody or antigen binding fragment thereof described herein (e.g., single chain variable fragment (scFv)) linked to T-cell signaling domains via a transmembrane domain. Characteristics of CARs include their ability to redirect T-cell specificity and reactivity toward a selected target in a non-MHC-restricted manner, and exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

In some embodiments, the antigen binding domain can include an scFv having at least one of:
a)
a CDR-H1 comprising the amino acid sequence of GFTFTDYY (SEQ ID NO: 3),
a CDR-H2 comprising the amino acid sequence of IRNKANGYRT (SEQ ID NO: 4), and
a CDR-H3 comprising the amino acid sequence of ASLSAY (SEQ ID NO: 5);
b)
a CDR-L1 comprising the amino acid sequence of KSLLHSNGITY (SEQ ID NO: 8),
a CDR-L2 comprising the amino acid sequence of QMS (SEQ ID NO: 9), and
a CDR-L3 comprising the amino acid sequence of AQNLELWT (SEQ ID NO: 10);
c)
a CDR-H1 comprising the amino acid sequence of GASITSGH (SEQ ID NO: 13),
a CDR-H2 comprising the amino acid sequence of IRDSGTT (SEQ ID NO: 14), and
a CDR-H3 comprising the amino acid sequence of VTTPFVY (SEQ ID NO: 15);
d)
a CDR-L1 comprising the amino acid sequence of QDINSY (SEQ ID NO: 18),
a CDR-L2 comprising the amino acid sequence of RAN (SEQ ID NO: 19), and
a CDR-L3 comprising the amino acid sequence of LQYDEFPLT (SEQ ID NO: 20);
e)
a CDR-H1 comprising the amino acid sequence of GFSLFNYG (SEQ ID NO: 23),
a CDR-H2 comprising the amino acid sequence of IWADGTI (SEQ ID NO: 24), and
a CDR-H3 comprising the amino acid sequence of ARHFDY (SEQ ID NO: 25);
f)
a CDR-L1 comprising the amino acid sequence of QNIVHSNGNTY (SEQ ID NO: 28),
a CDR-L2 comprising the amino acid sequence of KVS (SEQ ID NO: 29), and
a CDR-L3 comprising FQGSRLPLT (SEQ ID NO: 30);
g)
a CDR-H1 comprising the amino acid sequence of GFIFSSYY (SEQ ID NO: 33),
a CDR-H2 comprising the amino acid sequence of ININGGST (SEQ ID NO: 34), and
a CDR-H3 comprising the amino acid sequence of GRHDHGYGWFAY (SEQ ID NO: 35);
h)
a CDR-L1 comprising the amino acid sequence of QNVGRN (SEQ ID NO: 38),
a CDR-L2 comprising the amino acid sequence of SAS (SEQ ID NO: 39), and
a CDR-L3 comprising the amino acid sequence of QQYNSYPWT (SEQ ID NO: 40);
i)
a CDR-H1 comprising the amino acid sequence of GYTFTTAG (SEQ ID NO: 43),
a CDR-H2 comprising the amino acid sequence of INTQSGVS (SEQ ID NO: 44), and
a CDR-H3 comprising the amino acid sequence of ARSNSGGFPY (SEQ ID NO: 45),
j)
a CDR-L1 comprising the amino acid sequence QSLFNSTTHKIY (SEQ ID NO: 48),
a CDR-L2 comprising the amino acid sequence FAS (SEQ ID NO: 49), and
a CDR-L3 comprising the amino acid sequence of QQHYTAPWT (SEQ ID NO: 50);
k)
a CDR-H1 comprising the amino acid sequence of GYTFTTVG (SEQ ID NO: 53),
a CDR-H2 comprising the amino acid sequence of INTHSGVS (SEQ ID NO: 54), and
a CDR-H3 comprising the amino acid sequence of ARGNSNYGFPY (SEQ ID NO: 55);
l)
a CDR-L1 comprising the amino acid sequence of EDIYNR (SEQ ID NO: 58),
a CDR-L2 comprising the amino acid sequence of GAT (SEQ ID NO: 59), and
a CDR-L3 comprising the amino acid sequence of QQYWSTPWT (SEQ ID NO: 60);
m)
a CDR-H1 comprising the amino acid sequence of GYSITSDYA (SEQ ID NO: 63),
a CDR-H2 comprising the amino acid sequence of ISYRSIT (SEQ ID NO: 64), and
a CDR-H3 comprising the amino acid sequence of ARFGYDGNY (SEQ ID NO: 65);
n)
a CDR-L1 comprising the amino acid sequence of QNIVHSNGDTY (SEQ ID NO: 68),
a CDR-L2 comprising the amino acid sequence of RVS (SEQ ID NO: 69), and
a CDR-L3 comprising the amino acid sequence of FQGSHVPLT (SEQ ID NO: 70);
o)
a CDR-H1 comprising the amino acid sequence of GYTFTDYN (SEQ ID NO: 73),
a CDR-H2 comprising the amino acid sequence of INPDNGAT (SEQ ID NO: 74), and
a CDR-H3 comprising the amino acid sequence of VRGFDY (SEQ ID NO: 75);
p)
a CDR-L1 comprising the amino acid sequence of QAIVHSNGNTY (SEQ ID NO: 78),
a CDR-L2 comprising the amino acid sequence of KVS (SEQ ID NO: 79), and
a CDR-L3 comprising the amino acid sequence of FQSSHIPWT (SEQ ID NO: 80); or
a heavy chain variable region and/or light chain variable region that competitively inhibits binding of a peptide, protein, isolated antibody, antibody fragment or antigen binding fragment thereof comprising at least one of a), b), c), d), e), f), g), h), i), j), k), l), m), n), o), or p) to a CAM, such as PTPμ.

In other embodiments, the antigen binding domain can include a PTPμ ligand. The PTPμ ligand can include an amino acid sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 113.

The PTPμ ligand can be encoded by a nucleotide sequence at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to SEQ ID NO: 114.

In other embodiments, the PTPμ ligand can include a PTPμ partial sequence. A “partial sequence” or “PTPμ partial sequence” refers to a portion PTPμ that maintains PTPμ activity similar to that of the whole sequence, and in particular, an extracellular portion of PTPμ that is responsible for binding with PTPμ. In one example of a partial sequence is a sequence comprising at least 95%, 90%, 85%, 80%, 75%, 70%, 65%, 60%, 55%, 50%, 45%, 40%, 35%, 30%, 25%, 20%, 15% or 10% of the naturally occurring PTPμ sequence. Also contemplated are sequences having at least 90%, 95%, 96%, 97%, 98%, 99% or 100% amino acid sequence identity across the partial sequence. In one example, the polypeptide can include a BAFF ligand that has, for example, an amino acid sequence at least about 70%, at least about 75%, at least about 80%, at least about 85%, at least about 90%, or at least about 95% identical to SEQ ID NO: 113.

The CAR can further include a hinge region, a transmembrane domain, at least one co-stimulatory domain, and a signaling domain.

The hinge region is a sequence positioned between for example, the antigen binding domain, and at least one of a transmembrane domain, co-stimulatory domain, or a signaling domain. The hinge sequence may be obtained, for example, from any suitable sequence from any genus, including human or a part thereof. Such hinge regions are known in the art. In one embodiment, the hinge region includes the hinge region of a human protein including CD-8 alpha, CD28, 4-1BB, OX40, CD3-zeta, T cell receptor α or β chain, a CD3 zeta chain, CD28, CD3c, CD45, CD4, CD5, CD8, CD9, CD16, CD22, CD33, CD37, CD64, CD80, CD86, CD134, CD137, ICOS, CD154, functional derivatives thereof, and combinations thereof.

In some embodiments, the hinge region includes can include an immunoglobulin selected from IgG1, IgG2, IgG3, IgG4, or IgD.

The transmembrane domain includes a hydrophobic polypeptide that spans the cellular membrane. In particular, the transmembrane domain spans from one side of a cell membrane (extracellular) through to the other side of the cell membrane (intracellular or cytoplasmic).

The transmembrane domain may be in the form of an alpha helix or a beta barrel, or combinations thereof. The transmembrane domain may include a polytopic protein, which has many transmembrane segments, each alpha-helical, beta sheets, or combinations thereof.

In one embodiment, the transmembrane domain that naturally is associated with one of the domains in the CAR is used. In another embodiment, the transmembrane domain can be selected or modified by amino acid substitution to avoid binding of such domains to the transmembrane domains of the same or different surface membrane proteins to minimize interactions with other members of the receptor complex.

For example, a transmembrane domain includes a transmembrane domain of a T-cell receptor α or β chain, a CD3 zeta chain, CD28, CD3ε, CD45, CD4, CD5, CD8, CD9, CD16, CD22, CD33, CD37, CD64, CD80, CD86, CD134, CD137, ICOS, CD154, functional derivatives thereof, and combinations thereof.

The artificially designed transmembrane domain is a polypeptide mainly comprising hydrophobic residues, such as leucine and valine. In one embodiment, a triplet of phenylalanine, tryptophan and valine is found at each end of the synthetic transmembrane domain.

In one embodiment, the transmembrane domain is the CD8 transmembrane domain. In another embodiment, the transmembrane domain is the CD28 transmembrane domain. Such transmembrane domains are known in the art.

The signaling domain and co-stimulatory domain include polypeptides that provide activation of an immune cell to stimulate or activate at least some aspect of the immune cell signaling pathway.

In an embodiment, the signaling domain includes the polypeptide of a functional signaling domain of CD3 zeta, common FcR gamma (FCER1G), Fc gamma R11a, FcR beta (Fc Epsilon Rib), CD3 gamma, CD3 delta, CD3 epsilon, CD79a, CD79b, DNAX-activating protein 10 (DAP10), DNAX-activating protein 12 (DAP12), active fragments thereof, functional derivatives thereof, and combinations thereof. Such signaling domains are known in the art.

In an embodiment, the CAR further includes one or more co-stimulatory domains. In an embodiment, the co-stimulatory domain is a functional signaling domain(s) selected from at least a protein including, but not limited to, IL-15 receptor alpha; IL-15 receptor alpha cytoplasmic domain; B7-1/CD80; CD28; 4-1BB, 4-1BBL, B7-2/CD86; CTLA-4; B7-H1/PD-L1; ICOS; B7-H2; PD-1; B7-H3; PD-L2; B7-H4; PDCD6; BTLA; 4-1BB/TNFRSF9/CD137; CD40 Ligand/TNFSFS; 4-1BB Ligand/TNFSF9; GITR/TNFRSF18; BAFF/BLyS/TNFSF13B; GITR Ligand/TNFSF18; BAFF R/TNFRSF13C; HVEM/TNFRSF14; CD27/TNFRSF7; LIGHT/TNFSF14; CD27 Ligand/TNFSF7; OX40/TNFRSF4; CD30/TNFRSF8; OX40 Ligand/TNFSF4; CD30 Ligand/TNFSF8; TACI/TNFRSF13B; CD40/TNFRSF5; 2B4/CD244/SLAMF4; CD84/SLAMFS; BLAME/SLAMF8; CD229/SLAMF3; CD2, CD27, CRACC/SLAMF7; CD2F-10/SLAMF9; NTB-A/SLAMF6; CD48/SLAMF2; SLAM/CD150; CD58/LFA-3; Ikaros; CD53; Integrin alpha 4/CD49d; CD82/Kai-1; Integrin alpha 4 beta 1; CD90/Thy1; Integrin alpha 4 beta 7/LPAM-1; CD96; LAG-3; CD160; LMIR1/CD300A; CRTAM; TCL1A; DAP12; TIM-1/KIM-1/HAVCR; Dectin-1/CLEC7A; TIM-4; DPPIV/CD26; TSLP; EphB6; TSLP R; and HLA-DR.

Other embodiments relate to a nucleic acid comprising a nucleotide sequence encoding a CAR described herein. The nucleic acid encoding the CAR is easily prepared from an amino acid sequence of the specified CAR by any conventional method. A base sequence encoding an amino acid sequence can be obtained from the aforementioned NCBI RefSeq IDs or accession numbers of GenBank for an amino acid sequence of each domain, and the nucleic acid of the present disclosure can be prepared using a standard molecular biological and/or chemical procedure. For example, based on the base sequence, a polynucleotide can be synthesized, and the polynucleotide of the present disclosure can be prepared by combining DNA fragments which are obtained from a cDNA library using a polymerase chain reaction (PCR).

The CAR encoding nucleotide sequence can be operably linked to a promoter and provided in an expression construct. The vector can be suitable for replication and integration into eukaryotes. Typical vectors contain transcription and translation terminators, initiation sequences, and promoters useful for regulation of the expression of the desired nucleic acid sequence.

In an embodiment, the vector is a viral vector. Viral vector technology is known in the art and is described, for example, in Sambrook et al., 2012, Molecular Cloning: A Laboratory Manual, volumes 1-4, Cold Spring Harbor Press, NY), and in other virology and molecular biology manuals. In an embodiment, viruses, which are useful as vectors are retroviruses, adenoviruses, adeno-associated viruses, herpes viruses, and lentiviruses. In an embodiment the vector is a lentivirus vector. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193)

Vectors derived from viruses, e.g., lentivirus, are suitable tools to achieve long-term gene transfer since they allow long-term, stable integration of a transgene and its propagation in daughter cells. Lentiviral vectors have the added advantage over vectors derived from retroviruses e.g., murine leukemia viruses, in that they can transduce non-proliferating cells, such as hepatocytes. They also have the added advantage of low immunogenicity.

A retroviral vector may also be, e.g., a gammaretroviral vector. A gammaretroviral vector may include, e.g., a promoter, a packaging signal (T), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom.

In some embodiments, the vector can express two or more genes, where each gene is expressed separately under the control of a different promoter region, e.g., by using bi or tri-cistronic promoters. Expression of two or more genes from the same vector can be achieved by using either a multiple promoter plasmid e.g., bi or tri-cistronic promoters. Examples of multiple promoter containing lentivirus vectors are known in the literature. For example the vector pLENTI-bi-cistronic drives the expression of two genes using the PKG promoter and the mini CMV promoter in opposite directions (Applied Biological Material Inc., Richmond, BC, Canada). Similarly, the tri-cistronic vector pLENTI-tri-cistronic drives expression of three genes. In this configuration one gene can be induced by the mini-CMV promoter while the second and third gene can be induced by the PGK promoter separating the two genes with a T2A peptide cleavage site.

In another embodiment, bi- or tri-cistronic vectors may also be constructed making use of internal ribosomal entry sites (IRES) such as for example the element from the encephalomyocarditis virus (EMCV) for translation of two or more open reading frames (ORFs). Such vectors are designed to drive transcription of the bi- or tri-cistronic message under control of a strong human promoter regulatory region e.g., CMV or EF1alpha. IRESs are relatively short DNA sequences that can initiate RNA translation in a 5′ cap-independent fashion. Whereas the first cistron is translated in a cap-dependent manner driven by a strong mammalian promoter, the subsequent ones utilize intercistronic regions of viral origin such as the internal ribosomal entry site of poliovirus or the cap-independent translation enhancer of encephalomyocarditis virus for enhanced translation.

Additional promoter elements, e.g., enhancers, can regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, the individual elements can function either cooperatively or independently to activate transcription.

Other examples of promoters include an SFFV promoter and a cytomegalovirus (CMV) promoter sequence. Other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter (EF1α), the hemoglobin promoter, and the creatine kinase promoter.

Further, embodiments are not limited to the use of constitutive promoters and can include, for example, inducible promoters. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

The vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g., SV40 origin and ColE1 or others known in the art) and/or elements to allow selection (e.g., puromycin resistant gene, ampicillin resistance gene and/or zeocin marker).

Sequences encoding various elements of a CAR can be disposed on the same nucleic acid molecule, e.g., the same plasmid or vector, e.g., viral vector, e.g., lentiviral vector. For example, both (i) sequence encoding extracellular antigen binding domain and (ii) sequence encoding an intracellular signaling member, can be present on the same nucleic acid, e.g., vector. Production of the corresponding proteins can be achieved, e.g., by the use of separate promoters, or by the use of a bicistronic transcription product (which can result in the production of two proteins by cleavage of a single translation product or by the translation of two separate protein products).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors; in other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York). A preferred method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyi phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyi phosphatidylglycerol (“DMPG”) and other lipids may be obtained from Avanti Polar Lipids, Inc. (Birmingham, Ala.). Stock solutions of lipids in chloroform or chloroform/methanol can be stored at about −20° C. Chloroform is used as the only solvent since it is more readily evaporated than methanol.

“Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 19 1 Glycobiology 5; 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous polynucleotides into a host cell or otherwise expose a cell to the polynucleotide of the present disclosure, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the disclosure.

In another embodiment, the disclosure provides an engineered cell expressing the chimeric antigen receptor polypeptide described above or polynucleotide encoding for the same, and described above.

An “engineered cell” means any cell of any organism that is modified, transformed, or manipulated by addition or modification of a gene, a DNA or RNA sequence, or protein or polypeptide. Isolated cells, host cells, and genetically engineered cells of the present disclosure include isolated immune cells, such as NK cells and T cells that contain the DNA or RNA sequences encoding a chimeric antigen receptor or chimeric antigen receptor complex and express the chimeric receptor on the cell surface. Isolated host cells and engineered cells may be used, for example, for enhancing an NK cell activity or a T lymphocyte activity, treatment of cancer, and treatment of infectious diseases.

Any cell capable of expressing and/or capable of integrating the chimeric antigen receptor polypeptide, as disclosed herein, into its membrane may be used.

In some embodiments, the CAR can be expressed in at least one immune cell. In certain embodiments, the immune cell is a T cell, e.g., a CD8+ T cell (e.g., a CD8+ naive T cell, central memory T cell, or effector memory T cell), a CD4+ T cell, a natural killer T cell (NKT cells), a regulatory T cell (Treg), a stem cell memory T cell, a lymphoid progenitor cell a hematopoietic stem cell, a natural killer cell (NK cell) or a dendritic cell. In certain embodiments, the cells are monocytes or granulocytes, e.g., myeloid cells, macrophages, neutrophils, dendritic cells, mast cells, eosinophils, and/or basophils.

In some embodiments, the immune cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen specificity, type of antigen receptor, presence in a particular organ or compartment, marker or cytokine secretion profile, and/or degree of differentiation. Among the sub-types and subpopulations of T cells and/or of CD4+ and/or of CD8+ T cells are naive T (TN) cells, effector T cells (TEFF), memory T cells and sub-types thereof, such as stem cell memory T (TSCM), central memory T (TCM), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as Th1 cells, Th2 cells, Th3 cells, Th17 cells, Th9 cells, Th22 cells, follicular helper T cells, α/β T cells, and δ/γ T cells. In certain embodiments, any number of T cell lines available in the art, may be used.

The engineered cells may be obtained from peripheral blood, cord blood, bone marrow, tumor infiltrating lymphocytes, lymph node tissue, or thymus tissue. The host cells may include placental cells, embryonic stem cells, induced pluripotent stem cells, or hematopoietic stem cells. The cells may be obtained from humans, monkeys, chimpanzees, dogs, cats, mice, rats, and transgenic species thereof. The cells may be obtained from established cell lines.

The above cells may be obtained by any known means. The cells may be autologous, syngeneic, allogeneic, or xenogeneic to the recipient of the engineered cells.

In certain embodiments, T and NK cells are derived from human peripheral blood mononuclear cells (PBMC), leukapheresis products (PBSC), human embryonic stem cells (hESCs), induced pluripotent stem cells (iPSCs), bone marrow, or umbilical cord blood.

The immunotherapies described herein, particularly, the peptides, proteins, antibodies, antibody fragments, antigen binding fragments, and CAR immune cells can be used in methods and compositions for treating cancer. In particular embodiments, the immunotherapies can be used as treatment of virtually all types of cancers and pre-cancers (e.g., Myelodysplastic syndrome), including but not limited to carcinomas, sarcomas, melanomas, lymphomas, and leukemias, and having places of origin including but not limited to colon, prostate, brain, breast, liver, lung, pancreatic, bone, ovarian, skin, pancreatic, blood and others. The methods disclosed herein are contemplated for treatment of both metastatic cancers as well as primary tumor sites.

The immunotherapies can be included in a composition, such as a pharmaceutical composition, for immunotherapy, adoptive immunotherapy, and/or treating cancer or an infectious disease. The pharmaceutical composition as described herein can optionally include a pharmaceutically acceptable carrier. The active ingredients of the pharmaceutical composition at a minimum comprise the immunotherapy, e.g., peptides, proteins, antibodies, antibody fragments, antigen-binding fragments, and CAR immune cells as described herein. In some embodiments, the active ingredients of the pharmaceutical composition consist essentially of the peptides, proteins, antibodies, antibody fragments, antigen-binding fragments, and CAR immune cells as described herein. In some embodiments, the active ingredients of the pharmaceutical composition consist of the peptides, proteins, antibodies, antibody fragments, antigen-binding fragments, and CAR immune cells as described herein. Pharmaceutically acceptable carriers for peptide, protein or cell-based therapeutic formulation include saline and aqueous buffer solutions, Ringer's solution, and serum component, such as serum albumin, HDL and LDL. The terms such as “excipient”, “carrier”, “pharmaceutically acceptable carrier” or the like are used interchangeably herein.

In some embodiments, the pharmaceutical composition comprising immunotherapies as described herein can be a parenteral dose form. Since administration of parenteral dosage forms typically bypasses the patient's natural defenses against contaminants, the components apart from the immunotherapies themselves are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. Any of these can be added to the immunotherapy preparation prior to administration.

Suitable vehicles that can be used to provide parenteral dosage forms of immunotherapies as disclosed within are well known to those skilled in the art. Examples include, without limitation: saline solution; glucose solution; aqueous vehicles including but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

In some embodiments, the immunotherapy described herein can administered as a monotherapy, i.e., another treatment for the condition is not concurrently administered to the subject.

Modes of administration can include, for example intravenous (i.v.) injection or infusion. The compositions described herein can be administered to a patient transarterially, intratumorally, intranodally, or intramedullary. In some embodiments, the compositions of immunotherapy may be injected directly into a tumor, lymph node, or site of infection. In one embodiment, the compositions described herein are administered into a body cavity or body fluid (e.g., ascites, pleural fluid, peritoneal fluid, or cerebrospinal fluid).

The dosage of the above treatments to be administered to a patient will vary with the precise nature of the condition being treated and the recipient of the treatment. The scaling of dosages for human administration can be performed according to art-accepted practices.

In some embodiments, a single treatment regimen is required. In others, administration of one or more subsequent doses or treatment regimens can be performed. For example, after treatment biweekly for three months, treatment can be repeated once per month, for six months or a year or longer. In some embodiments, no additional treatments are administered following the initial treatment.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to administer further cells, discontinue treatment, resume treatment, or make other alterations to the treatment regimen. The dosage should not be so large as to cause adverse side effects, such as cytokine release syndrome. Generally, the dosage will vary with the age, condition, and sex of the patient and can be determined by one of skill in the art. The dosage can also be adjusted by the individual physician in the event of any complication.

In some embodiments, the immunotherapies described herein can be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The immunotherapy described herein and the at least one additional therapeutic agent can be administered simultaneously, in the same or in separate compositions, or sequentially. For sequential administration, the immunotherapy described herein can be administered first, and the additional agent can be administered second, or the order of administration can be reversed. The immunotherapy and/or other therapeutic agents, procedures or modalities can be administered during periods of active disorder, or during a period of remission or less active disease. The immunotherapy can be administered before another treatment, concurrently with the treatment, post-treatment, or during remission of the disorder.

When administered in combination, the immunotherapy and the additional agent (e.g., second or third agent), or all, can be administered in an amount or dose that is higher, lower or the same as the amount or dosage of each agent used individually, e.g., as a monotherapy. In certain embodiments, the administered amount or dosage of the immunotherapy, the additional agent (e.g., second or third agent), or all, is lower (e.g., at least 20%, at least 30%, at least 40%, or at least 50%) than the amount or dosage of each agent used individually. In other embodiments, the amount or dosage of the immunotherapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent individually required to achieve the same therapeutic effect. In further embodiments, the immunotherapy described herein can be used in a treatment regimen in combination with surgery, chemotherapy, radiation, an mTOR pathway inhibitor, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxan, fludarabine, rapamycin, mycophenolic acid, steroids, FR901228, cytokines, or a peptide vaccine.

In one embodiment, the immunotherapy described herein can be used in combination with a checkpoint inhibitor. Exemplary checkpoint inhibitors include anti-PD-1 inhibitors (Nivolumab, MK-3475, Pembrolizumab, Pidilizumab, AMP-224, AMP-514), anti-CTLA4 inhibitors (Ipilimumab and Tremelimumab), anti-PDL1 inhibitors (Atezolizumab, Avelomab, MSB0010718C, MED14736, and MPDL3280A), and anti-TIM3 inhibitors.

In one embodiment, the immunotherapy described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, tositumomab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®). Exemplary alkylating agents include, without limitation, nitrogen mustards, ethylenimine derivatives, alkyl sulfonates, nitrosoureas and triazenes): uracil mustard (Aminouracil Mustard®, Chlorethaminacil®, Demethyldopan®, Desmethyldopan®, Haemanthamine®, Nordopan®, Uracil Nitrogen Mustard®, Uracillost®, Uracilmostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HC1 (Treanda®). Exemplary mTOR inhibitors include, e.g., temsirolimus; ridaforolimus (formally known as deferolimus, (IR,2R,45)-4-[(2R)-2[(1R,95,125,15R,16E,18R,19R,21R,235,24E,26E,28Z,305,325,35R)-1,18-dihydroxy-19,30-dimethoxy-15,17,21,23,29,35-hexamethyl-2,3,10,14,20-pentaoxo-11,36-dioxa-4-azatricyclo[30.3.1.04′9]hexatriaconta-16,24,26,28-tetraen-12-yl]propyl]-2-methoxycyclohexyl dimethylphosphinate, also known as AP23573 and MK8669, and described in PCT Publication No. WO 03/064383); everolimus (Afinitor® or RADOOI); rapamycin (AY22989, Sirolimus®); simapimod (CAS 164301-51-3); emsirolimus, (5-{2,4-Bis[(35)-3-methylmorpholin-4-yl]pyrido[2,3-(i]pyrimidin-7-yl}-2-methoxyphenyl)methanol (AZD8055); 2-Amino-8-[iraw5,-4-(2-hydroxyethoxy)cyclohexyl]-6-(6-methoxy-3-pyridinyl)-4-methyl-pyrido[2,3-JJpyrimidin-7(8H)-one (PF04691502, CAS 1013101-36-4); and N2-[1,4-dioxo-4-[[4-(4-oxo-8-phenyl-4H-1-benzopyran-2-yl)morpholinium-4-yl]methoxy]butyl]L-arginylglycyl-L-a-aspartyl-L-serine-, inner salt (SF1126, CAS 936487-67-1), and XL765. Exemplary immunomodulators include, e.g., afutuzumab (available from Roche®); pegfilgrastim (Neulasta®); lenalidomide (CC-5013, Revlimid®); thalidomide (Thalomid®), actimid (CC4047); and IRX-2 (mixture of human cytokines including interleukin 1, interleukin 2, and interferon γ, CAS 951209-71-5, available from IRX Therapeutics). Exemplary anthracyclines include, e.g., doxorubicin (Adriamycin® and Rubex®); bleomycin (Ienoxane®); daunorubicin (dauorubicin hydrochloride, daunomycin, and rubidomycin hydrochloride, Cerubidine®); daunorubicin liposomal (daunorubicin citrate liposome, DaunoXome®); mitoxantrone (DHAD, Novantrone®); epirubicin (Ellence™); idarubicin (Idamycin®, Idamycin PFS®); mitomycin C (Mutamycin®); geldanamycin; herbimycin; ravidomycin; and desacetylravidomycin. Exemplary vinca alkaloids include, e.g., vinorelbine tartrate (Navelbine®), Vincristine (Oncovin®), and Vindesine (Eldisine®)); vinblastine (also known as vinblastine sulfate, vincaleukoblastine and VLB, Alkaban-AQ® and Velban®); and vinorelbine (Navelbine®). Exemplary proteosome inhibitors include bortezomib (Velcade®); carfilzomib (PX-171-007, (5)-4-Methyl-N-((5)-I-(((5)-4-methyl-I-((R)-2-methyloxiran-2-yl)-I-oxopentan-2-yl)amino)-I-oxo-3-phenylpropan-2-yl)-2-((5)-2-(2-morpholinoacetamido)-4-phenylbutanamido)-pentanamide); marizomib (NPT0052); ixazomib citrate (MLN-9708); delanzomib (CEP-18770); and O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912).

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloffs Clinical Oncology, 2013 Elsevier; and Fischer D S (ed): The Cancer Chemotherapy Handbook, 4th ed. St. Louis, Mosby-Year Book, 2003).

The efficacy of immunotherapy in, e.g., the treatment of a condition described herein, or to induce a response as described herein (e.g., a reduction in cancer cells) can be determined by the skilled clinician. However, a treatment is considered “effective treatment,” as the term is used herein, if one or more of the signs or symptoms of a condition described herein is altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate. Treatment according to the methods described herein can reduce levels of a marker or symptom of a condition, e.g., by at least 10%, at least 15%, at least 20%, at least 25%, at least 30%, at least 40%, at least 50%, at least 60%, at least 70%, at least 80% or at least 90% or more.

Efficacy can also be measured by a failure of an individual to worsen as assessed by hospitalization, or need for medical interventions (i.e., progression of the disease is halted). Methods of measuring these indicators are known to those of skill in the art and/or are described herein.

### EXAMPLE

We studied the role of cell-cell adhesion molecules (CAMs), predominately the immunoglobulin (Ig) superfamily and found that proteolysis of CAMs is a common event in cancer and cleaved fragments of CAMs function as tumor biomarkers. Full length CAMs including the Ig superfamily receptor protein tyrosine phosphatase, PTPμ, function as tumor suppressors and normally contribute to cell-cell adhesion by binding homophilically to PTPμ on adjacent cells to link cells together. In the microenvironment of multiple tumor types, PTPμ proteolysis creates an extracellular (ECD) fragment that leads to the formation of unique epitopes (neoantigens) expressed exclusively on the surface of tumor cells. We developed peptides, proteins and antibodies that bind to this novel tumor biomarker. The antibodies include humanized versions and scFv antibody fragments. One of these agents is a novel type of reagent called the PTPμ-Fc chimera (described below), which we originally utilized for adhesion and neurite outgrowth studies. Conjugating these agents to tags allows recognition and tracking of tumors using imaging. These agents are capable of labeling both the primary tumor and their invasive and metastatic cells especially in brain tumors.

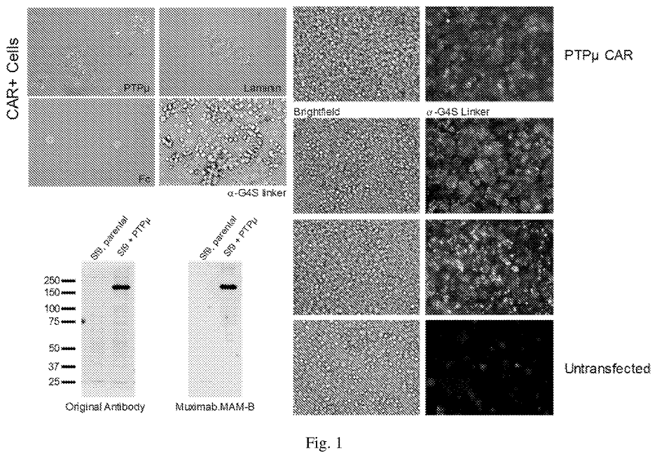

Fc domain chimeric proteins, in which the Fc domain of the human immunoglobulin heavy chain IgG1 is conjugated to a tumor specific protein, are a potential new class of immunotherapies. The Fc domain binds to Fcγ receptors (FcγR) on innate immune cells triggering antibody-dependent cellular cytotoxicity (ADCC) by natural killer (NK) cells and phagocytosis of tumor cells by macrophages in the periphery or microglia in the brain to recruit them to the tumor microenvironment to trigger an immune response. In addition, the FcγR on dendritic cells (DC) present tumor antigens to T cells. We explored two approaches to utilize these tumor specific targeting agents to target GBM. First, we utilized the PTPμ-Fc chimeric agent to stimulate the immune system to target and kill tumor cells. By combining the tumor targeting potential of PTPμ with the body's own immune recognition, we hypothesize that we will trigger ADCC as shown by our data with NK cells (FIG. 1). No therapeutic Fc fusions to CAMs have yet been developed and our studies of CAM cleavage as a mechanism to detect and target cancer are unique. The important innovation is that a “human” or “humanized” reagent is created because both the PTPμ and Fc sequences are derived from humans, which allows immediate translation to patients. Second, we utilized our existing human scFV and peptide sequences targeting PTPμ to generate novel immunotherapies.

We developed novel immunotherapies including Fc fusions to CAMs that have been developed for cell adhesion studies. In GBM, PTPμ proteolysis creates extracellular domain (ECD) fragments. Previously, we developed peptides and protein fragments that bind homophilically to this novel tumor biomarker and label both the primary mass and distant invasive glioma cells. By combining the GBM targeting potential of PTPμ with the body's own immune recognition of the Fc region, we hypothesized that the PTPμ-targeted agents will trigger ADCC. Exploiting PTPμ targeting can 1) home in on and identify tumor cells, 2) penetrate into the protected brain parenchyma 3) label both the main tumor and its invasive and metastatic cells equally well. We are developing these agents as tumor targeted immunotherapeutics. We hypothesize that the Fc or scFv component of the agent will add antibody functionality, yet have improved tumor tissue penetration, as it is substantially smaller than a typical antibody. The PTPμ targeting protein element will home in on and attach to the tumor-associated PTPμ ECD on tumor cells, thereby triggering: the innate immune system, including the complement response by Fc engaging with the complement receptor as well as antibody triggered cellular cytotoxicity and antibody triggered cellular phagocytosis by binding to FcγR on infiltrating immune cells; and the adaptive immune system, by triggering MHC class I antigen presentation on tumor cells and cytotoxic T lymphocyte attack of these tumor cells. These PTPμ agents have a strong potential to enhance NK cell activity through ADCC.

Sequences
Sequence: JM-F-scFv(H) Range: 1 to 345
(SEQ ID NO: 1)
EVKLVESGGGLVQPGGSLRLSCATSGFTFTDYYMSWVRQPPGKALEWLGFIRNKAN
GYRTEYSASVKGRFTISRDNSQSILYLQMNTLRAEDSATYYCASLSAYWGQGTLVTV
SA
(SEQ ID NO: 2)
GAGGTGAAGCTGGTGGAGTCTGGAGGAGGCTTGGTACAGCCTGGGGGTTCTCTG
AGACTCTCCTGTGCAACTTCTGGGTTCACCTTCACTGATTACTACATGAGCTGGG
TCCGCCAGCCTCCAGGAAAGGCACTTGAGTGGTTGGGTTTTATTAGAAACAAAG
CTAATGGTTACAGAACAGAGTACAGTGCATCTGTGAAGGGTCGGTTCACCATCTC
CAGAGATAATTCCCAAAGCATCCTCTATCTTCAAATGAACACCCTGAGAGCTGAG
GACAGTGCCACTTATTACTGTGCAAGCTTATCTGCTTACTGGGGCCAAGGGACTC
TGGTCACTGTCTCTGCA
(SEQ ID NO: 3)
GFTFTDYY
(SEQ ID NO: 4)
IRNKANGYRT
(SEQ ID NO: 5)
ASLSAY
Sequence: JM-F-scFv(H) Range: 1 to 333
(SEQ ID NO: 6)
DIVMTQAAFSNPVTLGTSASISCRSSKSLLHSNGITYLYWFLQKPGQSPQLLIYQMSN
LASGVPDRESSSGSGTDFTLRISRVEADDVGVYYCAQNLELWTFGGGTKLEIK
(SEQ ID NO: 7)
GATATTGTGATGACGCAGGCTGCATTCTCCAATCCAGTCACTCTTGGAACATCAG
CTTCCATCTCCTGCAGGTCTAGTAAGAGTCTCCTACATAGTAATGGCATCACTTA
TTTGTATTGGTTTCTGCAGAAGCCAGGCCAGTCTCCTCAG
CTCCTGATTTATCAGATGTCCAACCTTGCCTCAGGAGTCCCAGACAGGTTCAGTA
GCAGTGGGTCAGGAACTGATTTCACACTGAGAATCAGCAGAGTGGAGGCTGACG
ATGTGGGTGTTTATTACTGTGCTCAAAATCTAGAACTGTGGACGTTCGGTGGAGG
CACCAAGCTGGAAATCAAA
(SEQ ID NO: 8)
KSLLHSNGITY
(SEQ ID NO: 9)
QMS
(SEQ ID NO: 10)
AQNLELWT
Sequence: JM-G-scFv(H) Range: 1 to 339
(SEQ ID NO: 11)
EVQLQESGPSLVKPSQTLSLTCSVTGASITSGHWNWIRKFPGNKLEYMGYIRDSGTT
YYNPSLKGRLSITRDTSKNQYYLQLSSVTAEDTATYYCVTTPFVYWGQGTLVTVSA
(SEQ ID NO: 12)
GAGGTGCAGCTTCAGGAGTCAGGACCTAGCCTCGTGAAACCTTCTCAGACTCTGT
CCCTCACCTGTTCTGTCACTGGCGCCTCCATCACCAGTGGTCACTGGAACTGGAT
CCGGAAATTCCCTGGAAATAAACTTGAATATATGGGGTACATAAGGGACAGTGG
TACCACTTACTACAACCCATCTCTCAAAGGTCGACTCTCCATCACTCGAGACACA
TCCAAGAACCAGTACTACCTGCAGTTGAGTTCTGTGACTGCTGAGGACACAGCCA
CATATTATTGTGTGACTACGCCGTTTGTTTACTGGGGCCAAGGGACTCTGGTCAC
TGTCTCTGCA
(SEQ ID NO: 13)
GASITSGH
(SEQ ID NO: 14)
IRDSGTT
(SEQ ID NO: 15)
VTTPFVY
Sequence: JM-G-scFv(L) Range: 1 to 321
(SEQ ID NO: 16)
DIKMTQSPSSMFASLGERVTITCKASQDINSYLSWFQQKPGKSPKTLFYRANRLIDGV
PSRFSGSGSGQDYSLTISSLENEDMGIYYCLQYDEFPLTFGAGTKLELK
(SEQ ID NO: 17)
GACATCAAGATGACCCAGTCTCCATCTTCCATGTTTGCATCTCTAGGAGAGAGAG
TCACTATCACTTGCAAGGCGAGTCAGGACATTAATAGTTATTTAAGTTGGTTCCA
GCAGAAACCAGGGAAATCTCCTAAGACCCTATTCTATCGTGCAAATAGATTAAT
AGATGGGGTCCCATCAAGGTTCAGTGGCAGTGGATCTGGGCAAGATTATTCTCTC
ACCATCAGCAGCCTGGAGAATGAAGATATGGGAATTTATTACTGTCTACAGTATG
ATGAGTTTCCCCTCACGTTCGGTGCTGGGACCAAGCTGGAGCTGAAA
(SEQ ID NO: 18)
QDINSY
(SEQ ID NO: 19)
RAN
(SEQ ID NO: 20)
LQYDEFPLT
Sequence: MAM-B-scFv(H) Range: 1 to 336
(SEQ ID NO: 21)
QVHLKESGPGLVAPSQSLSITCTVSGFSLFNYGVHWVRQPPGKGLEWLGVIWADGTI
NYNSALMSRLTITKDNIKSQVFLKMDSLKTDDTAIYYCARHFDYWGQGTTLTVSS
(SEQ ID NO: 22)
CAGGTGCACCTGAAGGAGTCAGGACCTGGCCTGGTGGCGCCCTCACAGAGCCTG
TCCATCACTTGCACTGTCTCTGGCTTTTCATTATTCAACTATGGTGTACACTGGGT
TCGCCAGCCTCCAGGAAAGGGTCTGGAGTGGCTGGGAGTTATTTGGGCTGACGG
AACCATAAATTATAATTCGGCTCTCATGTCCAGACTGACCATCACCAAAGACAAC
ATCAAGAGCCAGGTTTTCTTAAAAATGGACAGTCTGAAAACTGACGACACAGCC
ATATACTACTGTGCCAGACACTTTGACTACTGGGGCCAAGGCACCACTCTCACAG
TCTCCTCA
(SEQ ID NO: 23)
GFSLFNYG
(SEQ ID NO: 24)
IWADGTI
(SEQ ID NO: 25)
ARHFDY
Sequence: MAM-B-scFv(L) Range: 1 to 336
DVLLTQIPLSLPVSLGDQASISCRASQNIVHSNGNTYLDWYLQKPGQSPKLLIYKVSN
(SEQ ID NO: 26)
RFSGVPDRFSGSGSGTDFTLKISRVEAEDLGVYYCFQGSRLPLTFGVGTKLELK
(SEQ ID NO: 27)
GATGTTTTGCTGACCCAAATTCCGCTCTCCCTGCCTGTCAGTCTTGGAGATCAAG
CCTCCATCTCCTGCAGAGCTAGTCAGAACATTGTACATAGTAATGGAAACACTTA
TTTAGATTGGTACCTGCAGAAACCAGGCCAGTCTCCAAAGCTCCTGATCTACAAA
GTTTCCAATCGATTTTCTGGGGTCCCAGACAGGTTCAGTGGCAGTGGATCAGGGA
CAGATTTCACACTCAAGATCAGCAGAGTGGAGGCTGAGGATCTGGGAGTTTATT
ATTGTTTTCAAGGTTCACGTCTTCCGCTCACGTTCGGTGTTGGGACCAAGCTGGA
GCTGAAA
(SEQ ID NO: 28)
QNIVHSNGNTY
(SEQ ID NO: 29)
KVS
(SEQ ID NO: 30)
FQGSRLPLT
Sequence: MAM-D-scFv(H) Range: 1 to 357
(SEQ ID NO: 31)
DVKLVESGGGLVKLGGSLKLSCAASGFIFSSYYMSWVRQTPEKKLELVAAININGGS
TYYPDTVKGRFTISRDNAKNTLYLQMSSLKSEDTALYYCGRHDHGYGWFAYWGQG
TLVTVSA
(SEQ ID NO: 32)
GACGTGAAGCTCGTGGAGTCGGGGGGAGGCTTAGTGAAACTTGGAGGGTCCCTG
AAACTCTCCTGTGCAGCCTCTGGATTCATTTTCAGTAGCTATTACATGTCTTGGGT
TCGCCAGACTCCGGAGAAGAAGCTGGAGTTGGTCGCAGCCATTAATATTAATGG
TGGTAGCACCTACTATCCAGACACTGTGAAGGGCCGATTCACCATCTCCAGAGAC
AATGCCAAGAACACCCTGTACCTGCAAATGAGCAGTCTGAAGTCTGAGGACACA
GCCTTATATTACTGTGGAAGACATGACCATGGGTACGGCTGGTTTGCTTACTGGG
GCCAAGGGACTCTGGTCACTGTCTCTGCA
(SEQ ID NO: 33)
GFIFSSYY
(SEQ ID NO: 34)
ININGGST
(SEQ ID NO: 35)
GRHDHGYGWFAY
Sequence: MAM-D-scFv(L) Range: 1 to 321
(SEQ ID NO: 36)
DIVMTQSQKVMSTSVGDRVSVTCKASQNVGRNIAWYQQKPGQSPKTLIYSASSRYS
GVPDRFTGSGFGTYFILTINNVQSEDLAAYFCQQYNSYPWTFGGGTKLEIK
GACATTGTGATGACCCAGTCTCAAAAAGTCATGTCCACATCAGTAGGA
(SEQ ID NO: 37)
GACAGGGTCAGCGTCACCTGCAAGGCCAGTCAGAATGTGGGTAGGAATATAGCC
TGGTATCAACAGAAACCAGGGCAATCTCCTAAAACACTGATATATTCGGCATCCT
CCCGGTACAGTGGAGTCCCTGATCGCTTCACAGGCAGTGGATTTGGGACATATTT
CATTCTCACCATCAACAACGTGCAGTCTGAAGACTTGGCAGCATATTTCTGTCAG
CAATATAATAGTTATCCGTGGACGTTCGGTGGAGGCACCAAGCTGGAAATCAAA
(SEQ ID NO: 38)
QNVGRN
(SEQ ID NO: 39)
SAS
(SEQ ID NO: 40)
QQYNSYPWT
Sequence: MAM-G-scFv(H) Range: 1 to 351
(SEQ ID NO:41)
QIQLVQSGPELKNPGETVRISCKASGYTFTTAGIQWVQKMPGKGLKWIGWINTQSGV
SKCAEDFKGRFALSMETYASTAYLQMSNLKKEDTATYFCARSNSGGFPYWGQGTL
VTVSA
(SEQ ID NO: 42)
CAAATCCAGTTGGTGCAGTCAGGACCTGAGCTGAAGAACCCTGGAGAGACAGTC
AGGATCTCCTGCAAGGCTTCTGGGTATACCTTCACAACTGCTGGAATCCAGTGGG
TGCAAAAGATGCCAGGAAAGGGTTTGAAGTGGATTGGCTGGATAAACACCCAGT
CTGGAGTGTCAAAATGTGCAGAAGACTTCAAGGGTCGATTTGCCTTGTCCATGGA
AACCTACGCCAGCACTGCATATTTACAGATGAGCAACCTCAAAAAAGAGGACAC
GGCTACGTATTTCTGTGCGAGATCGAACTCCGGCGGTTTTCCTTACTGGGGCCAA
GGGACTCTGGTCACTGTCTCTGCA
(SEQ ID NO: 43)
GYTFTTAG
(SEQ ID NO: 44)
INTQSGVS
(SEQ ID NO: 45)
ARSNSGGFPY
Sequence: MAM-G-scFv(L) Range: 1 to 339
(SEQ ID NO: 46)
DIVMTQSPSSLAMSIGQKVTMSCKSSQSLFNSTTHKIYLAWYQQKPGQSPKLLVYFA
STRQSGVPDRFIGSGSWTDFTLTINSVQAEDLADYFCQQHYTAPWTFGGGTKLEIR
(SEQ ID NO: 47)
GACATTGTGATGACACAGTCTCCATCCTCCCTGGCTATGTCAATTGGACAGAAGG
TCACTATGAGCTGCAAGTCCAGTCAGAGCCTTTTTAATAGTACCACTCACAAGAT
CTATTTGGCCTGGTACCAGCAGAAACCAGGACAGTCTCCTAAACTTCTGGTATAC
TTTGCATCCACTAGACAATCTGGGGTCCCTGATCGTTTCATCGGCAGTGGATCTT
GGACAGATTTCACTCTTACCATCAACAGTGTGCAGGCTGAAGACCTGGCAGATTA
CTTCTGTCAGCAACATTATACCGCTCCGTGGACGTTCGGTGGAGGCACC
AAGCTGGAAATCAGA
(SEQ ID NO: 48)
QSLFNSTTHKIY
(SEQ ID NO: 49)
FAS
(SEQ ID NO: 50)
QQHYTAPWT
Sequence: MAM-I-scFv(H) Range: 1 to 354
(SEQ ID NO: 51)
QIQLVQSGPELKKPGETVRISCKASGYTFTTVGMQWVQKMPGKGLRWIGWINTHSG
VSKYAEDFKGRFAFSLETSASTAYLEISNLKNEDTATYFCARGNSNYGFPYWGQGTL
VTVSA
(SEQ ID NO: 52)
CAGATCCAGTTGGTGCAGTCTGGACCTGAGCTGAAGAAGCCTGGAGAGACAGTC
AGGATCTCCTGCAAGGCTTCTGGGTATACCTTCACAACTGTTGGAATGCAGTGGG
TGCAAAAGATGCCAGGAAAGGGTTTGAGGTGGATTGGCTGGATAAACACCCACT
CTGGAGTGTCAAAATATGCAGAAGACTTCAAGGGACGGTTTGCCTTCTCTTTGGA
AACCTCTGCCAGCACTGCATATTTAGAGATAAGCAACCTCAAAAATGAGGACAC
GGCTACGTATTTCTGTGCGAGGGGAAATAGTAACTACGGGTTTCCTTACTGGGGC
CAAGGGACTCTGGTCACTGTCTCTGCA
(SEQ ID NO: 53)
GYTFTTVG
(SEQ ID NO: 54)
INTHSGVS
(SEQ ID NO: 55)
ARGNSNYGFPY
Sequence: MAM-I-scFv(L) Range: 1 to 321
(SEQ ID NO: 56)
DIQMTQSSSSFSVSLGDRVTITCKASEDIYNRLAWYQQRPGNAPRLLISGATTLETGV
PSRFSGSGSGKDYTLSITSLQTEDVATYYCQQYWSTPWTFGGGTKLEIK
(SEQ ID NO: 57)
GACATCCAGATGACACAATCTTCATCCTCCTTTTCTGTATCTCTAGGAGACAGAG
TCACCATTACTTGCAAGGCAAGTGAAGACATATATAATCGGTTAGCCTGGTATCA
GCAGAGACCAGGAAATGCTCCTAGGCTCTTAATATCTGGTGCAACCACTTTGGAA
ACTGGGGTTCCTTCAAGATTCAGTGGCAGTGGATCTGGAAAGGATTACACTCTCA
GCATTACCAGTCTTCAGACTGAAGATGTTGCTACTTATTACTGTCAACAATATTG
GAGTACTCCGTGGACGTTCGGTGGAGGCACCAAGCTGGAAATCAAA
(SEQ ID NO: 58)
EDIYNR
(SEQ ID NO: 59)
GAT
(SEQ ID NO: 60)
QQYWSTPWT
Sequence: PTPD1-O-scFv(H) Range: 1 to 348
(SEQ ID NO: 61)
DVQLQESGPGLVKPSQSLSLTCTVTGYSITSDYAWTWIRQFPGNKLEWMGYISYRSIT
GYNPSLKSRISINRDTSKNQFFLQLNSVTTEDTATYYCARFGYDGNYWGQGTTLTVS
S
(SEQ ID NO: 62)
GATGTGCAGCTTCAGGAGTCGGGACCTGGCCTGGTGAAACCTTCTCAGTCTCTGT
CCCTCACCTGCACTGTCACTGGCTACTCAATCACCAGTGATTATGCCTGGACCTG
GATCCGGCAGTTTCCCGGAAACAAACTGGAGTGGATGGGCTACATAAGCTACAG
AAGTATCACTGGCTACAACCCATCTCTCAAAAGTCGAATTTCTATCAATCGAGAC
ACATCCAAGAACCAGTTCTTCCTGCAGTTGAATTCTGTGACTACTGAGGACACAG
CCACATATTACTGTGCAAGATTTGGTTATGACGGGAACTACTGGGGCCAAGGCA
CCACTCTCACAGTCTCCTCA
(SEQ ID NO: 63)
GYSITSDYA
(SEQ ID NO: 64)
ISYRSIT
(SEQ ID NO: 65)
ARFGYDGNY
Sequence: PTPD1-O-scFv(L) Range: 1 to 336
(SEQ ID NO: 66)
DVLMTQTPLSLPVSLGDQASISCRSSQNIVHSNGDTYLEWYLQKPGQSPKLLIYRVSN
RFSGVPDRFSGSGSGTDFTLKISRVEAEDLGVYYCFQGSHVPLTFGAGTKLELK
(SEQ ID NO: 67)
GATGTTTTGATGACCCAAACTCCACTCTCCCTGCCTGTCAGTCTTGGAGATCAAG
CCTCCATCTCTTGCAGATCTAGTCAGAACATTGTACATAGTAATGGAGACACCTA
TTTAGAATGGTACCTGCAGAAACCAGGCCAGTCTCCAAAGCTCCTGATCTACAGA
GTTTCCAACCGATTTTCTGGGGTCCCAGACAGGTTCAGTGGCAGTGGATCAGGGA
CAGATTTCACACTCAAGATCAGCAGAGTGGAGGCTGAGGATCTGGGAGTTTATT
ACTGCTTTCAAGGTTCACATGTTCCGCTCACGTTCGGTGCTGGGACCAAGCTG
GAGCTGAAA
(SEQ ID NO: 68)
QNIVHSNGDTY
(SEQ ID NO: 69)
RVS
(SEQ ID NO: 70)
FQGSHVPLT
Sequence: PTPD1-R-scFv(H) Range: 1 to 339
(SEQ ID NO: 71)
EVLLQQSGPELVKPGTSVKIPCKASGYTFTDYNMDWVKQSHGKSLEWIGDINPDNG
ATVYNQKFKGKATLTVDKSSSTAYMELRSLTSEDTAVYYCVRGFDYWGQGTSVTV
SS
(SEQ ID NO: 72)
GAGGTCCTGCTGCAACAGTCTGGACCTGAGCTGGTGAAGCCTGGGACTTCAGTG
AAGATACCCTGCAAGGCTTCTGGATACACATTCACTGACTACAACATGGACTGG
GTGAAGCAGAGCCATGGAAAGAGCCTTGAGTGGATTGGAGATATTAATCCTGAC
AATGGTGCTACTGTCTACAACCAGAAGTTCAAGGGCAAGGCCACATTGACTGTT
GACAAGTCCTCCAGCACAGCCTACATGGAGCTCCGCAGCCTGACATCTGAGGAC
ACTGCAGTCTATTACTGTGTAAGAGGGTTCGACTACTGGGGTCAAGGAACCTCAG
TCACCGTCTCCTCA
(SEQ ID NO: 73)
GYTFTDYN
(SEQ ID NO: 74)
INPDNGAT
(SEQ ID NO: 75)
VRGFDY
Sequence: PTPD1-R-scFv(L) Range: 1 to 336
(SEQ ID NO: 76)
DVLMTQTPLSLPVSLGDQASISCRSSQAIVHSNGNTYLEWFLQKPGQSPKLLVYKVS
NRFSGVPDRFSGSGSGTDFTLRISRVEAEDLGVYYCFQSSHIPWTFGGGTKLEIK
(SEQ ID NO: 77)
GATGTTTTGATGACCCAGACTCCACTCTCCCTGCCTGTCAGTCTTGGAGATCAAG
CCTCCATCTCGTGCAGATCTAGTCAGGCCATTGTACATAGTAATGGAAACACCTA
TTTAGAATGGTTCCTGCAGAAACCAGGCCAGTCTCCAAAGCTCCTGGTCTACAAA
GTTTCCAACCGATTTTCTGGGGTCCCAGACAGGTTCAGTGGCAGTGGATCAGGGA
CAGATTTCACACTCAGGATCAGCAGAGTGGAGGCTGAGGATCTGGGAGTTTATT
ACTGCTTTCAGAGTTCTCATATTCCGTGGACGTTCGGTGGAGGCACCAAGCTGGA
AATCAAA
(SEQ ID NO: 78)
QAIVHSNGNTY
(SEQ ID NO: 79)
KVS
(SEQ ID NO: 80)
FQSSHIPWT
Sequence: Muximab-JM-F-Ckappa Range: 1 to 723
(SEQ ID NO: 81)
MDMRVPAQLLGLLLLWLRGARCDIVMTQAAFSNPVTLGTSASISCRSSKSLLHSNGI
TYLYWFLQKPGQSPQLLIYQMSNLASGVPDRESSSGSGTDFTLRISRVEADDVGVYY
CAQNLELWTFGGGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEVTHQGLS
SPVTKSFNRGEC
(SEQ ID NO: 82)
ATGGACATGAGAGTGCCCGCCCAGCTGCTGGGACTGCTGCTGCTGTGGCTGAGA
GGAGCGCGCTGCGATATTGTGATGACGCAGGCTGCATTCTCCAATCCAGTCACTC
TTGGAACATCAGCTTCCATCTCCTGCAGGTCTAGTAAGAGTCTCCTACATAGTAA
TGGCATCACTTATTTGTATTGGTTTCTGCAGAAGCCAGGCCAGTCTCCTCAGCTCC
TGATTTATCAGATGTCCAACCTTGCCTCAGGAGTCCCAGACAGGTTCAGTAGCAG
TGGGTCAGGAACTGATTTCACACTGAGAATCAGCAGAGTGGAGGCTGACGATGT
GGGTGTTTATTACTGTGCTCAAAATCTAGAACTGTGGACGTTCGGTGGAGGCACC
AAGCTGGAAATCAAACGTACGGTGGCCGCCCCCTCCGTGTTTATCTTCCCTCCCA
GCGACGAGCAGCTGAAGAGCGGCACAGCCAGCGTGGTGTGTCTGCTGAACAACT
TCTACCCTAGAGAGGCCAAGGTGCAGTGGAAAGTGGACAACGCTCTGCAGTCCG
GCAACAGCCAAGAGAGCGTGACCGAGCAAGACAGCAAGGACAGCACCTACTCT
CTGAGCAGCACACTGACACTGTCCAAGGCCGACTACGAGAAGCACAAGGTGTAC
GCTTGCGAGGTGACCCACCAAGGACTGAGCAGCCCCGTGACCAAGAGCTTCAAT
AGGGGCGAGTGC TGA
Sequence: Muximab-JM-G-Ckappa Range: 1 to 711
(SEQ ID NO: 83)
MDMRVPAQLLGLLLLWLRGARCDIKMTQSPSSMFASLGERVTITCKASQDINSYLS
WFQQKPGKSPKTLFYRANRLIDGVPSRFSGSGSGQDYSLTISSLENEDMGIYYCLQYD
EFPLTFGAGTKLELKRTVAAPSVFIFPPSDEQLKSGTASVVCLLNNFYPREAKVQWK
VDNALQSGNSQESVTEQDSKDSTYSLSSTLTLSKADYEKHKVYACEVTHQGLSSPVT
KSFNRGEC
(SEQ ID NO: 84)
ATGGACATGAGAGTGCCCGCCCAGCTGCTGGGACTGCTGCTGCTGTGGCTGAGA
GGAGCGCGCTGCGACATCAAGATGACCCAGTCTCCATCTTCCATGTTTGCATCTC
TAGGAGAGAGAGTCACTATCACTTGCAAGGCGAGTCAGGACATTAATAGTTATTT
AAGTTGGTTCCAGCAGAAACCAGGGAAATCTCCTAAGACCCTATTCTATCGTGCA
AATAGATTAATAGATGGGGTCCCATCAAGGTTCAGTGGCAGTGGATCTGGGCAA
GATTATTCTCTCACCATCAGCAGCCTGGAGAATGAAGATATGGGAATTTATTACT
GTCTACAGTATGATGAGTTTCCCCTCACGTTCGGTGCTGGGACCAAGCTGGAGCT
GAAACGTACGGTGGCCGCCCCCTCCGTGTTTATCTTCCCTCCCAGCGACGAGCAG
CTGAAGAGCGGCACAGCCAGCGTGGTGTGTCTGCTGAACAACTTCTACCCTAGA
GAGGCCAAGGTGCAGTGGAAAGTGGACAACGCTCTGCAGTCCGGCAACAGCCAA
GAGAGCGTGACCGAGCAAGACAGCAAGGACAGCACCTACTCTCTGAGCAGCACA
CTGACACTGTCCAAGGCCGACTACGAGAAGCACAAGGTGTACGCTTGCGAGGTG
ACCCACCAAGGACTGAGCAGCCCCGTGACCAAGAGCTTCAATAGGGGCGAGTGC
TGA
Sequence: Muximab-MAM-B-Ckappa Range: 1 to 726
(SEQ ID NO: 85)
MDMRVPAQLLGLLLLWLRGARCDVLLTQIPLSLPVSLGDQASISCRASQNIVHSNGN
TYLDWYLQKPGQSPKLLIYKVSNRFSGVPDRFSGSGSGTDFTLKISRVEAEDLGVYY
CFQGSRLPLTFGVGTKLELKRTVAAPSVFIFPPSDEQLKSGTASVVCLLNNFYPREAK
VQWKVDNALQSGNSQESVTEQDSKDSTYSLSSTLTLSKADYEKHKVYACEVTHQGL
SSPVTKSFNRGEC
(SEQ ID NO: 86)
ATGGACATGAGAGTGCCCGCCCAGCTGCTGGGACTGCTGCTGCTGTGGCTGAGA
GGAGCGCGCTGCGATGTTTTGCTGACCCAAATTCCGCTCTCCCTGCCTGTCAGTC
TTGGAGATCAAGCCTCCATCTCCTGCAGAGCTAGTCAGAACATTGTACATAGTAA
TGGAAACACTTATTTAGATTGGTACCTGCAGAAACCAGGCCAGTCTCCAAAGCTC
CTGATCTACAAAGTTTCCAATCGATTTTCTGGGGTCCCAGACAGGTTCAGTGGCA
GTGGATCAGGGACAGATTTCACACTCAAGATCAGCAGAGTGGAGGCTGAGGATC
TGGGAGTTTATTATTGTTTTCAAGGTTCACGTCTTCCGCTCACGTTCGGTGTTGGG
ACCAAGCTGGAGCTGAAACGTACGGTGGCCGCCCCCTCCGTGTTTATCTTCCCTC
CCAGCGACGAGCAGCTGAAGAGCGGCACAGCCAGCGTGGTGTGTCTGCTGAACA
ACTTCTACCCTAGAGAGGCCAAGGTGCAGTGGAAAGTGGACAACGCTCTGCAGT
CCGGCAACAGCCAAGAGAGCGTGACCGAGCAAGACAGCAAGGACAGCACCTAC
TCTCTGAGCAGCACACTGACACTGTCCAAGGCCGACTACGAGAAGCACAAGGTG
TACGCTTGCGAGGTGACCCACCAAGGACTGAGCAGCCCCGTGACCAAGAGCTTC
AATAGGGGCGAG TGCTGA
Sequence: Muximab-MAM-D-Ckappa Range: 1 to 711
(SEQ ID NO: 87)
MDMRVPAQLLGLLLLWLRGARCDIVMTQSQKVMSTSVGDRVSVTCKASQNVGRNI
AWYQQKPGQSPKTLIYSASSRYSGVPDRFTGSGFGTYFILTINNVQSEDLAAYFCQQY
NSYPWTFGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPV
TKSFNRGEC
(SEQ ID NO: 88)
ATGGACATGAGAGTGCCCGCCCAGCTGCTGGGACTGCTGCTGCTGTGGCTGAGA
GGAGCGCGCTGCGACATTGTGATGACCCAGTCTCAAAAAGTCATGTCCACATCA
GTAGGAGACAGGGTCAGCGTCACCTGCAAGGCCAGTCAGAATGTGGGTAGGAAT
ATAGCCTGGTATCAACAGAAACCAGGGCAATCTCCTAAAACACTGATATATTCG
GCATCCTCCCGGTACAGTGGAGTCCCTGATCGCTTCACAGGCAGTGGATTTGGGA
CATATTTCATTCTCACCATCAACAACGTGCAGTCTGAAGACTTGGCAGCATATTT
CTGTCAGCAATATAATAGTTATCCGTGGACGTTCGGTGGAGGCACCAAGCTGGA
AATCAAACGTACGGTGGCCGCCCCCTCCGTGTTTATCTTCCCTCCCAGCGACGAG
CAGCTGAAGAGCGGCACAGCCAGCGTGGTGTGTCTGCTGAACAACTTCTACCCT
AGAGAGGCCAAGGTGCAGTGGAAAGTGGACAACGCTCTGCAGTCCGGCAACAG
CCAAGAGAGCGTGACCGAGCAAGACAGCAAGGACAGCACCTACTCTCTGAGCAG
CACACTGACACTGTCCAAGGCCGACTACGAGAAGCACAAGGTGTACGCTTGCGA
GGTGACCCACCAAGGACTGAGCAGCCCCGTGACCAAGAGCTTCAATAGGGGCGA
GTGCTGA
Sequence: Muximab-MAM-G-Ckappa Range: 1 to 729
(SEQ ID NO: 89)
MDMRVPAQLLGLLLLWLRGARCDIVMTQSPSSLAMSIGQKVTMSCKSSQSLFNSTT
HKIYLAWYQQKPGQSPKLLVYFASTRQSGVPDRFIGSGSWTDFTLTINSVQAEDLAD
YFCQQHYTAPWTFGGGTKLEIRRTVAAPSVFIFPPSDEQLKSGTASVVCLLNNFYPRE
AKVQWKVDNALQSGNSQESVTEQDSKDSTYSLSSTLTLSKADYEKHKVYACEVTH
QGLSSPVTKSFNRGEC
(SEQ ID NO: 90)
ATGGACATGAGAGTGCCCGCCCAGCTGCTGGGACTGCTGCTGCTGTGGCTGAGA
GGAGCGCGCTGCGACATTGTGATGACACAGTCTCCATCCTCCCTGGCTATGTCAA
TTGGACAGAAGGTCACTATGAGCTGCAAGTCCAGTCAGAGCCTTTTTAATAGTAC
CACTCACAAGATCTATTTGGCCTGGTACCAGCAGAAACCAGGACAGTCTCCTAA
ACTTCTGGTATACTTTGCATCCACTAGACAATCTGGGGTCCCTGATCGTTTCATCG
GCAGTGGATCTTGGACAGATTTCACTCTTACCATCAACAGTGTGCAGGCTGAAGA
CCTGGCAGATTACTTCTGTCAGCAACATTATACCGCTCCGTGGACGTTCGGTGGA
GGCACCAAGCTGGAAATCAGACGTACGGTGGCCGCCCCCTCCGTGTTTATCTTCC
CTCCCAGCGACGAGCAGCTGAAGAGCGGCACAGCCAGCGTGGTGTGTCTGCTGA
ACAACTTCTACCCTAGAGAGGCCAAGGTGCAGTGGAAAGTGGACAACGCTCTGC
AGTCCGGCAACAGCCAAGAGAGCGTGACCGAGCAAGACAGCAAGGACAGCACC
TACTCTCTGAGCAGCACACTGACACTGTCCAAGGCCGACTACGAGAAGCACAAG
GTGTACGCTTGCGAGGTGACCCACCAAGGACTGAGCAGCCCCGTGACCAAGAGC
TTCAATAGGGGC GAGTGCTGA
Sequence: Muximab-MAM-I-Ckappa Range: 1 to 711
(SEQ ID NO: 91)
MDMRVPAQLLGLLLLWLRGARCDIQMTQSSSSFSVSLGDRVTITCKASEDIYNRLA
WYQQRPGNAPRLLISGATTLETGVPSRFSGSGSGKDYTLSITSLQTEDVATYYCQQY
WSTPWTFGGGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPV
TKSFNRGEC
(SEQ ID NO:92)
ATGGACATGAGAGTGCCCGCCCAGCTGCTGGGACTGCTGCTGCTGTGGCTGAGA
GGAGCGCGCTGCGACATCCAGATGACACAATCTTCATCCTCCTTTTCTGTATCTCT
AGGAGACAGAGTCACCATTACTTGCAAGGCAAGTGAAGACATATATAATCGGTT
AGCCTGGTATCAGCAGAGACCAGGAAATGCTCCTAGGCTCTTAATATCTGGTGCA
ACCACTTTGGAAACTGGGGTTCCTTCAAGATTCAGTGGCAGTGGATCTGGAAAG
GATTACACTCTCAGCATTACCAGTCTTCAGACTGAAGATGTTGCTACTTATTACT
GTCAACAATATTGGAGTACTCCGTGGACGTTCGGTGGAGGCACCAAGCTGGAAA
TCAAACGTACGGTGGCCGCCCCCTCCGTGTTTATCTTCCCTCCCAGCGACGAGCA
GCTGAAGAGCGGCACAGCCAGCGTGGTGTGTCTGCTGAACAACTTCTACCCTAG
AGAGGCCAAGGTGCAGTGGAAAGTGGACAACGCTCTGCAGTCCGGCAACAGCCA
AGAGAGCGTGACCGAGCAAGACAGCAAGGACAGCACCTACTCTCTGAGCAGCAC
ACTGACACTGTCCAAGGCCGACTACGAGAAGCACAAGGTGTACGCTTGCGAGGT
GACCCACCAAGGACTGAGCAGCCCCGTGACCAAGAGCTTCAATAGGGGCGAGTG
CTGA
Sequence: Muximab-PTPD1-O-Ckappa Range: 1 to 726
(SEQ ID NO: 93)
MDMRVPAQLLGLLLLWLRGARCDVLMTQTPLSLPVSLGDQASISCRSSQNIVHSNG
DTYLEWYLQKPGQSPKLLIYRVSNRFSGVPDRFSGSGSGTDFTLKISRVEAEDLGVY
YCFQGSHVPLTFGAGTKLELKRTVAAPSVFIFPPSDEQLKSGTASVVCLLNNFYPREA
KVQWKVDNALQSGNSQESVTEQDSKDSTYSLSSTLTLSKADYEKHKVYACEVTHQ
GLSSPVTKSFNRGEC
(SEQ ID NO: 94)
ATGGACATGAGAGTGCCCGCCCAGCTGCTGGGACTGCTGCTGCTGTGGCTGAGA
GGAGCGCGCTGCGATGTTTTGATGACCCAAACTCCACTCTCCCTGCCTGTCAGTC
TTGGAGATCAAGCCTCCATCTCTTGCAGATCTAGTCAGAACATTGTACATAGTAA
TGGAGACACCTATTTAGAATGGTACCTGCAGAAACCAGGCCAGTCTCCAAAGCT
CCTGATCTACAGAGTTTCCAACCGATTTTCTGGGGTCCCAGACAGGTTCAGTGGC
AGTGGATCAGGGACAGATTTCACACTCAAGATCAGCAGAGTGGAGGCTGAGGAT
CTGGGAGTTTATTACTGCTTTCAAGGTTCACATGTTCCGCTCACGTTCGGTGCTGG
GACCAAGCTGGAGCTGAAACGTACGGTGGCCGCCCCCTCCGTGTTTATCTTCCCT
CCCAGCGACGAGCAGCTGAAGAGCGGCACAGCCAGCGTGGTGTGTCTGCTGAAC
AACTTCTACCCTAGAGAGGCCAAGGTGCAGTGGAAAGTGGACAACGCTCTGCAG
TCCGGCAACAGCCAAGAGAGCGTGACCGAGCAAGACAGCAAGGACAGCACCTA
CTCTCTGAGCAGCACACTGACACTGTCCAAGGCCGACTACGAGAAGCACAAGGT
GTACGCTTGCGAGGTGACCCACCAAGGACTGAGCAGCCCCGTGACCAAGAGCTT
CAATAGGGGCGAG TGCTGA
Sequence: Muximab-PTPD1-R-Ckappa Range: 1 to 726
(SEQ ID NO: 95)
MDMRVPAQLLGLLLLWLRGARCDVLMTQTPLSLPVSLGDQASISCRSSQAIVHSNG
NTYLEWFLQKPGQSPKLLVYKVSNRFSGVPDRFSGSGSGTDFTLRISRVEAEDLGVY
YCFQSSHIPWTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACEVTHQGL
SSPVTKSFNRGEC
(SEQ ID NO:96)
ATGGACATGAGAGTGCCCGCCCAGCTGCTGGGACTGCTGCTGCTGTGGCTGAGA
GGAGCGCGCTGCGATGTTTTGATGACCCAGACTCCACTCTCCCTGCCTGTCAGTC
TTGGAGATCAAGCCTCCATCTCGTGCAGATCTAGTCAGGCCATTGTACATAGTAA
TGGAAACACCTATTTAGAATGGTTCCTGCAGAAACCAGGCCAGTCTCCAAAGCTC
CTGGTCTACAAAGTTTCCAACCGATTTTCTGGGGTCCCAGACAGGTTCAGTGGCA
GTGGATCAGGGACAGATTTCACACTCAGGATCAGCAGAGTGGAGGCTGAGGATC
TGGGAGTTTATTACTGCTTTCAGAGTTCTCATATTCCGTGGACGTTCGGTGGAGG
CACCAAGCTGGAAATCAAACGTACGGTGGCCGCCCCCTCCGTGTTTATCTTCCCT
CCCAGCGACGAGCAGCTGAAGAGCGGCACAGCCAGCGTGGTGTGTCTGCTGAAC
AACTTCTACCCTAGAGAGGCCAAGGTGCAGTGGAAAGTGGACAACGCTCTGCAG
TCCGGCAACAGCCAAGAGAGCGTGACCGAGCAAGACAGCAAGGACAGCACCTA
CTCTCTGAGCAGCACACTGACACTGTCCAAGGCCGACTACGAGAAGCACAAGGT
GTACGCTTGCGAGGTGACCCACCAAGGACTGAGCAGCCCCGTGACCAAGAGCTT
CAATAGGGGCGAG TGCTGA
Sequence: Muximab-JM-F-IGG1 Range: 1 to 1395
(SEQ ID NO: 97)
MEFGLSWVFLVAILKGVQCEVKLVESGGGLVQPGGSLRLSCATSGFTFTDYYMSWV
RQPPGKALEWLGFIRNKANGYRTEYSASVKGRFTISRDNSQSILYLQMNTLRAEDSA
TYYCASLSAYWGQGTLVTVSAASTKGPSVFPLAPSSKSTSGGTAALGCLVKDYFPEP
VTVSWNSGALTSGVHTFPAVLQSSGLYSLSSVVTVPSSSLGTQTYICNVNHKPSNTK
VDKKVEPKSCDKTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKEYKCK
VSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNH
YTQKSLSLSPGK
(SEQ ID NO: 98)
ATGGAGTTCGGACTGAGCTGGGTGTTTCTGGTGGCCATTCTGAAGGGTGTACAGT
GCGAGGTGAAGCTGGTGGAGTCTGGAGGAGGCTTGGTACAGCCTGGGGGTTCTC
TGAGACTCTCCTGTGCAACTTCTGGGTTCACCTTCACTGATTACTACATGAGCTG
GGTCCGCCAGCCTCCAGGAAAGGCACTTGAGTGGTTGGGTTTTATTAGAAACAA
AGCTAATGGTTACAGAACAGAGTACAGTGCATCTGTGAAGGGTCGGTTCACCAT
CTCCAGAGATAATTCCCAAAGCATCCTCTATCTTCAAATGAACACCCTGAGAGCT
GAGGACAGTGCCACTTATTACTGTGCAAGCTTATCTGCTTACTGGGGCCAAGGGA
CTCTGGTCACTGTCTCTGCAGCTAGCACCAAGGGCCCTAGCGTGTTTCCTCTGGC
CCCCTCCAGCAAGAGCACAAGCGGAGGAACCGCTGCTCTGGGATGTCTGGTGAA
GGATTACTTCCCCGAGCCCGTGACAGTGAGCTGGAACAGCGGAGCTCTGACCTC
CGGCGTGCATACATTTCCCGCCGTGCTGCAGAGCAGCGGCCTCTACTCTCTGTCC
AGCGTGGTGACCGTGCCTAGCTCCTCTCTGGGAACACAGACCTACATCTGCAACG
TGAACCACAAGCCCTCCAACACCAAGGTCGATAAGAAGGTGGAGCCCAAAAGCT
GCGACAAGACACACACATGTCCCCCTTGCCCCGCTCCCGAGCTGCTGGGAGGAC
CCTCCGTGTTTCTGTTTCCCCCCAAGCCCAAGGACACACTGATGATCAGCAGAAC
ACCCGAGGTGACATGCGTGGTGGTGGATGTCAGCCACGAGGACCCCGAGGTCAA
GTTCAATTGGTACGTGGACGGCGTGGAGGTGCACAATGCCAAGACCAAGCCCAG
AGAGGAGCAGTACAACTCCACATATAGAGTGGTGAGCGTGCTGACCGTGCTGCA
CCAAGACTGGCTGAACGGCAAGGAATACAAGTGCAAGGTCTCCAACAAGGCTCT
GCCCGCCCCCATCGAGAAGACAATCTCCAAGGCCAAAGGCCAGCCCAGAGAGCC
CCAAGTCTACACACTGCCTCCCTCTAGGGATGAGCTGACCAAGAACCAAGTGAG
CCTCACATGTCTGGTGAAGGGCTTCTATCCCAGCGACATCGCTGTGGAGTGGGAG
AGCAACGGCCAGCCCGAGAACAACTACAAGACCACACCCCCCGTGCTGGACAGC
GATGGCTCCTTCTTTCTGTACTCCAAGCTGACCGTGGACAAATCTAGGTGGCAAC
AAGGCAACGTGTTTAGCTGCTCCGTGATGCACGAGGCCCTCCACAACCACTATAC
CCAGAAGTCTCTGTCTCTGTCCCCCGGCAAGTGA
Sequence: Muximab-JM-G-IGG1 Range: 1 to 1389
(SEQ ID NO: 99)
MEFGLSWVFLVAILKGVQCEVQLQESGPSLVKPSQTLSLTCSVTGASITSGHWNWIR
KFPGNKLEYMGYIRDSGTTYYNPSLKGRLSITRDTSKNQYYLQLSSVTAEDTATYYC
VTTPFVYWGQGTLVTVSAASTKGPSVFPLAPSSKSTSGGTAALGCLVKDYFPEPVTV
SWNSGALTSGVHTFPAVLQSSGLYSLSSVVTVPSSSLGTQTYICNVNHKPSNTKVDK
KVEPKSCDKTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKCKVSN
KALPAPIEKTISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEALHNHYTQ
KSLSLSPGK
(SEQ ID NO: 100)
ATGGAGTTCGGACTGAGCTGGGTGTTTCTGGTGGCCATTCTGAAGGGTGTACAGT
GCGAGGTGCAGCTTCAGGAGTCAGGACCTAGCCTCGTGAAACCTTCTCAGACTCT
GTCCCTCACCTGTTCTGTCACTGGCGCCTCCATCACCAGTGGTCACTGGAACTGG
ATCCGGAAATTCCCTGGAAATAAACTTGAATATATGGGGTACATAAGGGACAGT
GGTACCACTTACTACAACCCATCTCTCAAAGGTCGACTCTCCATCACTCGAGACA
CATCCAAGAACCAGTACTACCTGCAGTTGAGTTCTGTGACTGCTGAGGACACAGC
CACATATTATTGTGTGACTACGCCGTTTGTTTACTGGGGCCAAGGGACTCTGGTC
ACTGTCTCTGCAGCTAGCACCAAGGGCCCTAGCGTGTTTCCTCTGGCCCCCTCCA
GCAAGAGCACAAGCGGAGGAACCGCTGCTCTGGGATGTCTGGTGAAGGATTACT
TCCCCGAGCCCGTGACAGTGAGCTGGAACAGCGGAGCTCTGACCTCCGGCGTGC
ATACATTTCCCGCCGTGCTGCAGAGCAGCGGCCTCTACTCTCTGTCCAGCGTGGT
GACCGTGCCTAGCTCCTCTCTGGGAACACAGACCTACATCTGCAACGTGAACCAC
AAGCCCTCCAACACCAAGGTCGATAAGAAGGTGGAGCCCAAAAGCTGCGACAA
GACACACACATGTCCCCCTTGCCCCGCTCCCGAGCTGCTGGGAGGACCCTCCGTG
TTTCTGTTTCCCCCCAAGCCCAAGGACACACTGATGATCAGCAGAACACCCGAGG
TGACATGCGTGGTGGTGGATGTCAGCCACGAGGACCCCGAGGTCAAGTTCAATT
GGTACGTGGACGGCGTGGAGGTGCACAATGCCAAGACCAAGCCCAGAGAGGAG
CAGTACAACTCCACATATAGAGTGGTGAGCGTGCTGACCGTGCTGCACCAAGAC
TGGCTGAACGGCAAGGAATACAAGTGCAAGGTCTCCAACAAGGCTCTGCCCGCC
CCCATCGAGAAGACAATCTCCAAGGCCAAAGGCCAGCCCAGAGAGCCCCAAGTC
TACACACTGCCTCCCTCTAGGGATGAGCTGACCAAGAACCAAGTGAGCCTCACA
TGTCTGGTGAAGGGCTTCTATCCCAGCGACATCGCTGTGGAGTGGGAGAGCAAC
GGCCAGCCCGAGAACAACTACAAGACCACACCCCCCGTGCTGGACAGCGATGGC
TCCTTCTTTCTGTACTCCAAGCTGACCGTGGACAAATCTAGGTGGCAACAAGGCA
ACGTGTTTAGCTGCTCCGTGATGCACGAGGCCCTCCACAACCACTATACCCAGAA
GTCTCTGTCTCTGTCCCCCGGCAAGTGA
Sequence: Muximab-MAM-B-IGG1 Range: 1 to 1386
(SEQ ID NO: 101)
MEFGLSWVFLVAILKGVQCQVHLKESGPGLVAPSQSLSITCTVSGFSLFNYGVHWVR
QPPGKGLEWLGVIWADGTINYNSALMSRLTITKDNIKSQVFLKMDSLKTDDTAIYYC
ARHFDYWGQGTTLTVSSASTKGPSVFPLAPSSKSTSGGTAALGCLVKDYFPEPVTVS
WNSGALTSGVHTFPAVLQSSGLYSLSSVVTVPSSSLGTQTYICNVNHKPSNTKVDKK
VEPKSCDKTHTCPPCPAPELLGGPSVFLFPPKPKDTLMISRTPEVTCVVVDVSHEDPE
VKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIA VEWESN
GQPENNYKTTPPVLDSDGSFFLYSKLTVDKSRWQQGNVFSCSVMHEALHNHYTQKS
LSLSPGK
(SEQ ID NO: 102)
ATGGAGTTCGGACTGAGCTGGGTGTTTCTGGTGGCCATTCTGAAGGGTGTACAGT
GCCAGGTGCACCTGAAGGAGTCAGGACCTGGCCTGGTGGCGCCCTCACAGAGCC
TGTCCATCACTTGCACTGTCTCTGGCTTTTCATTATTCAACTATGGTGTACACTGG
GTTCGCCAGCCTCCAGGAAAGGGTCTGGAGTGGCTGGGAGTTATTTGGGCTGAC
GGAACCATAAATTATAATTCGGCTCTCATGTCCAGACTGACCATCACCAAAGACA
ACATCAAGAGCCAGGTTTTCTTAAAAATGGACAGTCTGAAAACTGACGACACAG
CCATATACTACTGTGCCAGACACTTTGACTACTGGGGCCAAGGCACCACTCTCAC
AGTCTCCTCAGCTAGCACCAAGGGCCCTAGCGTGTTTCCTCTGGCCCCCTCCAGC
AAGAGCACAAGCGGAGGAACCGCTGCTCTGGGATGTCTGGTGAAGGATTACTTC
CCCGAGCCCGTGACAGTGAGCTGGAACAGCGGAGCTCTGACCTCCGGCGTGCAT
ACATTTCCCGCCGTGCTGCAGAGCAGCGGCCTCTACTCTCTGTCCAGCGTGGTGA
CCGTGCCTAGCTCCTCTCTGGGAACACAGACCTACATCTGCAACGTGAACCACAA
GCCCTCCAACACCAAGGTCGATAAGAAGGTGGAGCCCAAAAGCTGCGACAAGAC
ACACACATGTCCCCCTTGCCCCGCTCCCGAGCTGCTGGGAGGACCCTCCGTGTTT
CTGTTTCCCCCCAAGCCCAAGGACACACTGATGATCAGCAGAACACCCGAGGTG
ACATGCGTGGTGGTGGATGTCAGCCACGAGGACCCCGAGGTCAAGTTCAATTGG
TACGTGGACGGCGTGGAGGTGCACAATGCCAAGACCAAGCCCAGAGAGGAGCA
GTACAACTCCACATATAGAGTGGTGAGCGTGCTGACCGTGCTGCACCAAGACTG
GCTGAACGGCAAGGAATACAAGTGCAAGGTCTCCAACAAGGCTCTGCCCGCCCC
CATCGAGAAGACAATCTCCAAGGCCAAAGGCCAGCCCAGAGAGCCCCAAGTCTA
CACACTGCCTCCCTCTAGGGATGAGCTGACCAAGAACCAAGTGAGCCTCACATG
TCTGGTGAAGGGCTTCTATCCCAGCGACATCGCTGTGGAGTGGGAGAGCAACGG
CCAGCCCGAGAACAACTACAAGACCACACCCCCCGTGCTGGACAGCGATGGCTC
CTTCTTTCTGTACTCCAAGCTGACCGTGGACAAATCTAGGTGGCAACAAGGCAAC
GTGTTTAGCTGCTCCGTGATGCACGAGGCCCTCCACAACCACTATACCCAGAAGT
CTCTGTCTCTGTCCCCCGGCAAGTGA
Sequence: Muximab-MAM-D-IGG1 Range: 1 to 1407
(SEQ ID NO: 103)
MEFGLSWVFLVAILKGVQCDVKLVESGGGLVKLGGSLKLSCAASGFIFSSYYMSWV
RQTPEKKLELVAAININGGSTYYPDTVKGRFTISRDNAKNTLYLQMSSLKSEDTALY
YCGRHDHGYGWFAYWGQGTLVTVSAASTKGPSVFPLAPSSKSTSGGTAALGCLVK
DYFPEPVTVSWNSGALTSGVHTFPAVLQSSGLYSLSSVVTVPSSSLGTQTYICNVNHK
PSNTKVDKKVEPKSCDKTHTCPPCPAPELLGGPSVFLFPPKPKDTLMISRTPEVTCVV
VDVSHEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTCLVKGFYPS
DIAVEWESNGQPENNYKTTPPVLDSDGSFFLYSKLTVDKSRWQQGNVFSCSVMHEA
LHNHYTQKSLSLSPGK
(SEQ ID NO: 104)
ATGGAGTTCGGACTGAGCTGGGTGTTTCTGGTGGCCATTCTGAAGGGTGTACAGT
GCGACGTGAAGCTCGTGGAGTCGGGGGGAGGCTTAGTGAAACTTGGAGGGTCCC
TGAAACTCTCCTGTGCAGCCTCTGGATTCATTTTCAGTAGCTATTACATGTCTTGG
GTTCGCCAGACTCCGGAGAAGAAGCTGGAGTTGGTCGCAGCCATTAATATTAAT
GGTGGTAGCACCTACTATCCAGACACTGTGAAGGGCCGATTCACCATCTCCAGA
GACAATGCCAAGAACACCCTGTACCTGCAAATGAGCAGTCTGAAGTCTGAGGAC
ACAGCCTTATATTACTGTGGAAGACATGACCATGGGTACGGCTGGTTTGCTTACT
GGGGCCAAGGGACTCTGGTCACTGTCTCTGCAGCTAGCACCAAGGGCCCTAGCG
TGTTTCCTCTGGCCCCCTCCAGCAAGAGCACAAGCGGAGGAACCGCTGCTCTGGG
ATGTCTGGTGAAGGATTACTTCCCCGAGCCCGTGACAGTGAGCTGGAACAGCGG
AGCTCTGACCTCCGGCGTGCATACATTTCCCGCCGTGCTGCAGAGCAGCGGCCTC
TACTCTCTGTCCAGCGTGGTGACCGTGCCTAGCTCCTCTCTGGGAACACAGACCT
ACATCTGCAACGTGAACCACAAGCCCTCCAACACCAAGGTCGATAAGAAGGTGG
AGCCCAAAAGCTGCGACAAGACACACACATGTCCCCCTTGCCCCGCTCCCGAGC
TGCTGGGAGGACCCTCCGTGTTTCTGTTTCCCCCCAAGCCCAAGGACACACTGAT
GATCAGCAGAACACCCGAGGTGACATGCGTGGTGGTGGATGTCAGCCACGAGGA
CCCCGAGGTCAAGTTCAATTGGTACGTGGACGGCGTGGAGGTGCACAATGCCAA
GACCAAGCCCAGAGAGGAGCAGTACAACTCCACATATAGAGTGGTGAGCGTGCT
GACCGTGCTGCACCAAGACTGGCTGAACGGCAAGGAATACAAGTGCAAGGTCTC
CAACAAGGCTCTGCCCGCCCCCATCGAGAAGACAATCTCCAAGGCCAAAGGCCA
GCCCAGAGAGCCCCAAGTCTACACACTGCCTCCCTCTAGGGATGAGCTGACCAA
GAACCAAGTGAGCCTCACATGTCTGGTGAAGGGCTTCTATCCCAGCGACATCGCT
GTGGAGTGGGAGAGCAACGGCCAGCCCGAGAACAACTACAAGACCACACCCCC
CGTGCTGGACAGCGATGGCTCCTTCTTTCTGTACTCCAAGCTGACCGTGGACAAA
TCTAGGTGGCAACAAGGCAACGTGTTTAGCTGCTCCGTGATGCACGAGGCCCTCC
ACAACCACTATACCCAGAAGTCTCTGTCTCTGTCCCCCGGCAAGTGA
Sequence: Muximab-MAM-G-IGG1 Range: 1 to 1401
(SEQ ID NO: 105)
MEFGLSWVFLVAILKGVQCQIQLVQSGPELKNPGETVRISCKASGYTFTTAGIQWVQ
KMPGKGLKWIGWINTQSGVSKCAEDFKGRFALSMETYASTAYLQMSNLKKEDTAT
YFCARSNSGGFPYWGQGTLVTVSAASTKGPSVFPLAPSSKSTSGGTAALGCLVKDYF
PEPVTVSWNSGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHN
HYTQKSLSLSPGK
(SEQ ID NO: 106)
ATGGAGTTCGGACTGAGCTGGGTGTTTCTGGTGGCCATTCTGAAGGGTGTACAGT
GCCAAATCCAGTTGGTGCAGTCAGGACCTGAGCTGAAGAACCCTGGAGAGACAG
TCAGGATCTCCTGCAAGGCTTCTGGGTATACCTTCACAACTGCTGGAATCCAGTG
GGTGCAAAAGATGCCAGGAAAGGGTTTGAAGTGGATTGGCTGGATAAACACCCA
GTCTGGAGTGTCAAAATGTGCAGAAGACTTCAAGGGTCGATTTGCCTTGTCCATG
GAAACCTACGCCAGCACTGCATATTTACAGATGAGCAACCTCAAAAAAGAGGAC
ACGGCTACGTATTTCTGTGCGAGATCGAACTCCGGCGGTTTTCCTTACTGGGGCC
AAGGGACTCTGGTCACTGTCTCTGCAGCTAGCACCAAGGGCCCTAGCGTGTTTCC
TCTGGCCCCCTCCAGCAAGAGCACAAGCGGAGGAACCGCTGCTCTGGGATGTCT
GGTGAAGGATTACTTCCCCGAGCCCGTGACAGTGAGCTGGAACAGCGGAGCTCT
GACCTCCGGCGTGCATACATTTCCCGCCGTGCTGCAGAGCAGCGGCCTCTACTCT
CTGTCCAGCGTGGTGACCGTGCCTAGCTCCTCTCTGGGAACACAGACCTACATCT
GCAACGTGAACCACAAGCCCTCCAACACCAAGGTCGATAAGAAGGTGGAGCCCA
AAAGCTGCGACAAGACACACACATGTCCCCCTTGCCCCGCTCCCGAGCTGCTGG
GAGGACCCTCCGTGTTTCTGTTTCCCCCCAAGCCCAAGGACACACTGATGATCAG
CAGAACACCCGAGGTGACATGCGTGGTGGTGGATGTCAGCCACGAGGACCCCGA
GGTCAAGTTCAATTGGTACGTGGACGGCGTGGAGGTGCACAATGCCAAGACCAA
GCCCAGAGAGGAGCAGTACAACTCCACATATAGAGTGGTGAGCGTGCTGACCGT
GCTGCACCAAGACTGGCTGAACGGCAAGGAATACAAGTGCAAGGTCTCCAACAA
GGCTCTGCCCGCCCCCATCGAGAAGACAATCTCCAAGGCCAAAGGCCAGCCCAG
AGAGCCCCAAGTCTACACACTGCCTCCCTCTAGGGATGAGCTGACCAAGAACCA
AGTGAGCCTCACATGTCTGGTGAAGGGCTTCTATCCCAGCGACATCGCTGTGGAG
TGGGAGAGCAACGGCCAGCCCGAGAACAACTACAAGACCACACCCCCCGTGCTG
GACAGCGATGGCTCCTTCTTTCTGTACTCCAAGCTGACCGTGGACAAATCTAGGT
GGCAACAAGGCAACGTGTTTAGCTGCTCCGTGATGCACGAGGCCCTCCACAACC
ACTATACCCAGAAGTCTCTGTCTCTGTCCCCCGGCAAGTGA
Sequence: Muximab-MAM-I-IGG1 Range: 1 to 1404
(SEQ ID NO: 107)
MEFGLSWVFLVAILKGVQCQIQLVQSGPELKKPGETVRISCKASGYTFTTVGMQWV
QKMPGKGLRWIGWINTHSGVSKYAEDFKGRFAFSLETSASTAYLEISNLKNEDTATY
FCARGNSNYGFPYWGQGTLVTVSAASTKGPSVFPLAPSSKSTSGGTAALGCLVKDYF
PEPVTVSWNSGALTSGVHTFPAVLQSSGLYSLSSVVTVPSSSLGTQTYICNVNHKPSN
TKVDKKVEPKSCDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVKGFYPSDIA
VEWESNGQPENNYKTTPPVLDSDGSFFLYSKLTVDKSRWQQGNVFSCSVMHEALHN
HYTQKSLSLSPGK
(SEQ ID NO: 108)
ATGGAGTTCGGACTGAGCTGGGTGTTTCTGGTGGCCATTCTGAAGGGTGTACAGT
GCCAGATCCAGTTGGTGCAGTCTGGACCTGAGCTGAAGAAGCCTGGAGAGACAG
TCAGGATCTCCTGCAAGGCTTCTGGGTATACCTTCACAACTGTTGGAATGCAGTG
GGTGCAAAAGATGCCAGGAAAGGGTTTGAGGTGGATTGGCTGGATAAACACCCA
CTCTGGAGTGTCAAAATATGCAGAAGACTTCAAGGGACGGTTTGCCTTCTCTTTG
GAAACCTCTGCCAGCACTGCATATTTAGAGATAAGCAACCTCAAAAATGAGGAC
ACGGCTACGTATTTCTGTGCGAGGGGAAATAGTAACTACGGGTTTCCTTACTGGG
GCCAAGGGACTCTGGTCACTGTCTCTGCAGCTAGCACCAAGGGCCCTAGCGTGTT
TCCTCTGGCCCCCTCCAGCAAGAGCACAAGCGGAGGAACCGCTGCTCTGGGATG
TCTGGTGAAGGATTACTTCCCCGAGCCCGTGACAGTGAGCTGGAACAGCGGAGC
TCTGACCTCCGGCGTGCATACATTTCCCGCCGTGCTGCAGAGCAGCGGCCTCTAC
TCTCTGTCCAGCGTGGTGACCGTGCCTAGCTCCTCTCTGGGAACACAGACCTACA
TCTGCAACGTGAACCACAAGCCCTCCAACACCAAGGTCGATAAGAAGGTGGAGC
CCAAAAGCTGCGACAAGACACACACATGTCCCCCTTGCCCCGCTCCCGAGCTGCT
GGGAGGACCCTCCGTGTTTCTGTTTCCCCCCAAGCCCAAGGACACACTGATGATC
AGCAGAACACCCGAGGTGACATGCGTGGTGGTGGATGTCAGCCACGAGGACCCC
GAGGTCAAGTTCAATTGGTACGTGGACGGCGTGGAGGTGCACAATGCCAAGACC
AAGCCCAGAGAGGAGCAGTACAACTCCACATATAGAGTGGTGAGCGTGCTGACC
GTGCTGCACCAAGACTGGCTGAACGGCAAGGAATACAAGTGCAAGGTCTCCAAC
AAGGCTCTGCCCGCCCCCATCGAGAAGACAATCTCCAAGGCCAAAGGCCAGCCC
AGAGAGCCCCAAGTCTACACACTGCCTCCCTCTAGGGATGAGCTGACCAAGAAC
CAAGTGAGCCTCACATGTCTGGTGAAGGGCTTCTATCCCAGCGACATCGCTGTGG
AGTGGGAGAGCAACGGCCAGCCCGAGAACAACTACAAGACCACACCCCCCGTGC
TGGACAGCGATGGCTCCTTCTTTCTGTACTCCAAGCTGACCGTGGACAAATCTAG
GTGGCAACAAGGCAACGTGTTTAGCTGCTCCGTGATGCACGAGGCCCTCCACAA
CCACTATACCCAGAAGTCTCTGTCTCTGTCCCCCGGCAAGTGA
Sequence: Muximab-PTPD1-O-IGG1 Range: 1 to 1398
(SEQ ID NO: 109)
MEFGLSWVFLVAILKGVQCDVQLQESGPGLVKPSQSLSLTCTVTGYSITSDYAWTWI
RQFPGNKLEWMGYISYRSITGYNPSLKSRISINRDTSKNQFFLQLNSVTTEDTATYYC
ARFGYDGNYWGQGTTLTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYT
QKSLSLSPGK
(SEQ ID NO: 110)
ATGGAGTTCGGACTGAGCTGGGTGTTTCTGGTGGCCATTCTGAAGGGTGTACAGT
GCGATGTGCAGCTTCAGGAGTCGGGACCTGGCCTGGTGAAACCTTCTCAGTCTCT
GTCCCTCACCTGCACTGTCACTGGCTACTCAATCACCAGTGATTATGCCTGGACC
TGGATCCGGCAGTTTCCCGGAAACAAACTGGAGTGGATGGGCTACATAAGCTAC
AGAAGTATCACTGGCTACAACCCATCTCTCAAAAGTCGAATTTCTATCAATCGAG
ACACATCCAAGAACCAGTTCTTCCTGCAGTTGAATTCTGTGACTACTGAGGACAC
AGCCACATATTACTGTGCAAGATTTGGTTATGACGGGAACTACTGGGGCCAAGG
CACCACTCTCACAGTCTCCTCAGCTAGCACCAAGGGCCCTAGCGTGTTTCCTCTG
GCCCCCTCCAGCAAGAGCACAAGCGGAGGAACCGCTGCTCTGGGATGTCTGGTG
AAGGATTACTTCCCCGAGCCCGTGACAGTGAGCTGGAACAGCGGAGCTCTGACC
TCCGGCGTGCATACATTTCCCGCCGTGCTGCAGAGCAGCGGCCTCTACTCTCTGT
CCAGCGTGGTGACCGTGCCTAGCTCCTCTCTGGGAACACAGACCTACATCTGCAA
CGTGAACCACAAGCCCTCCAACACCAAGGTCGATAAGAAGGTGGAGCCCAAAAG
CTGCGACAAGACACACACATGTCCCCCTTGCCCCGCTCCCGAGCTGCTGGGAGG
ACCCTCCGTGTTTCTGTTTCCCCCCAAGCCCAAGGACACACTGATGATCAGCAGA
ACACCCGAGGTGACATGCGTGGTGGTGGATGTCAGCCACGAGGACCCCGAGGTC
AAGTTCAATTGGTACGTGGACGGCGTGGAGGTGCACAATGCCAAGACCAAGCCC
AGAGAGGAGCAGTACAACTCCACATATAGAGTGGTGAGCGTGCTGACCGTGCTG
CACCAAGACTGGCTGAACGGCAAGGAATACAAGTGCAAGGTCTCCAACAAGGCT
CTGCCCGCCCCCATCGAGAAGACAATCTCCAAGGCCAAAGGCCAGCCCAGAGAG
CCCCAAGTCTACACACTGCCTCCCTCTAGGGATGAGCTGACCAAGAACCAAGTG
AGCCTCACATGTCTGGTGAAGGGCTTCTATCCCAGCGACATCGCTGTGGAGTGGG
AGAGCAACGGCCAGCCCGAGAACAACTACAAGACCACACCCCCCGTGCTGGACA
GCGATGGCTCCTTCTTTCTGTACTCCAAGCTGACCGTGGACAAATCTAGGTGGCA
ACAAGGCAACGTGTTTAGCTGCTCCGTGATGCACGAGGCCCTCCACAACCACTAT
ACCCAGAAGTCTCTGTCTCTGTCCCCCGGCAAGTGA
Sequence: Muximab-PTPD1-R-IGG1 Range: 1 to 1389
(SEQ ID NO: 111)
MEFGLSWVFLVAILKGVQCEVLLQQSGPELVKPGTSVKIPCKASGYTFTDYNMDWV
KQSHGKSLEWIGDINPDNGATVYNQKFKGKATLTVDKSSSTAYMELRSLTSEDTAV
YYCVRGFDYWGQGTSVTVSSASTKGPSVFPLAPSSKSTSGGTAALGCLVKDYFPEPV
TVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVEW
ESNGQPENNYKTTPPVLDSDGSFFLYSKLTVDKSRWQQGNVFSCSVMHEALHNHYT
QKSLSLSPGK
(SEQ ID NO: 112)
ATGGAGTTCGGACTGAGCTGGGTGTTTCTGGTGGCCATTCTGAAGGGTGTACAGT
GCGAGGTCCTGCTGCAACAGTCTGGACCTGAGCTGGTGAAGCCTGGGACTTCAG
TGAAGATACCCTGCAAGGCTTCTGGATACACATTCACTGACTACAACATGGACTG
GGTGAAGCAGAGCCATGGAAAGAGCCTTGAGTGGATTGGAGATATTAATCCTGA
CAATGGTGCTACTGTCTACAACCAGAAGTTCAAGGGCAAGGCCACATTGACTGTT
GACAAGTCCTCCAGCACAGCCTACATGGAGCTCCGCAGCCTGACATCTGAGGAC
ACTGCAGTCTATTACTGTGTAAGAGGGTTCGACTACTGGGGTCAAGGAACCTCAG
TCACCGTCTCCTCAGCTAGCACCAAGGGCCCTAGCGTGTTTCCTCTGGCCCCCTC
CAGCAAGAGCACAAGCGGAGGAACCGCTGCTCTGGGATGTCTGGTGAAGGATTA
CTTCCCCGAGCCCGTGACAGTGAGCTGGAACAGCGGAGCTCTGACCTCCGGCGT
GCATACATTTCCCGCCGTGCTGCAGAGCAGCGGCCTCTACTCTCTGTCCAGCGTG
GTGACCGTGCCTAGCTCCTCTCTGGGAACACAGACCTACATCTGCAACGTGAACC
ACAAGCCCTCCAACACCAAGGTCGATAAGAAGGTGGAGCCCAAAAGCTGCGACA
AGACACACACATGTCCCCCTTGCCCCGCTCCCGAGCTGCTGGGAGGACCCTCCGT
GTTTCTGTTTCCCCCCAAGCCCAAGGACACACTGATGATCAGCAGAACACCCGAG
GTGACATGCGTGGTGGTGGATGTCAGCCACGAGGACCCCGAGGTCAAGTTCAAT
TGGTACGTGGACGGCGTGGAGGTGCACAATGCCAAGACCAAGCCCAGAGAGGA
GCAGTACAACTCCACATATAGAGTGGTGAGCGTGCTGACCGTGCTGCACCAAGA
CTGGCTGAACGGCAAGGAATACAAGTGCAAGGTCTCCAACAAGGCTCTGCCCGC
CCCCATCGAGAAGACAATCTCCAAGGCCAAAGGCCAGCCCAGAGAGCCCCAAGT
CTACACACTGCCTCCCTCTAGGGATGAGCTGACCAAGAACCAAGTGAGCCTCAC
ATGTCTGGTGAAGGGCTTCTATCCCAGCGACATCGCTGTGGAGTGGGAGAGCAA
CGGCCAGCCCGAGAACAACTACAAGACCACACCCCCCGTGCTGGACAGCGATGG
CTCCTTCTTTCTGTACTCCAAGCTGACCGTGGACAAATCTAGGTGGCAACAAGGC
AACGTGTTTAGCTGCTCCGTGATGCACGAGGCCCTCCACAACCAC
TATACCCAGAAGTCTCTGTCTCTGTCCCCCGGCAAGTGA
Sequence: ptprm AAs 21-621 Range: 61 to 1863
(SEQ ID NO: 113)
ETFSGGCLFDEPYSTCGYSQSEGDDFNWEQVNTLTKPTSDPWMPSGSFMLVNASGR
PEGQRAHLLLPQLKENDTHCIDFHYFVSSKSNSPPGLLNVYVKVNNGPLGNPIWNISG
DPTRTWNRAELAISTFWPNFYQVIFEVITSGHQGYLAIDEVKVLGHPCTRTPHFLRIQ
NVEVNAGQFATFQCSAIGRTVAGDRLWLQGIDVRDAPLKEIKVTSSRRFIASFNVVN
TTKRDAGKYRCMIRTEGGVGISNYAELVVKEPPVPIAPPQLASVGATYLWIQLNANSI
NGDGPIVAREVEYCTASGSWNDRQPVDSTSYKIGHLDPDTEYEISVLLTRPGEGGTG
SPGPALRTRTKCADPMRGPRKLEVVEVKSRQITIRWEPFGYNVTRCHSYNLTVHYCY
QVGGQEQVREEVSWDTENSHPQHTITNLSPYTNVSVKLILMNPEGRKESQELIVQTD
EDLPGAVPTESIQGSTFEEKIFLQWREPTQTYGVITLYEITYKAVSSFDPEIDLSNQSGR
VSKLGNETHFLFFGLYPGTTYSFTIRASTAKGFGPPATNQFTTKISAPSMPAYELETPL
NQTDNTVTVMLKPAHSRGAPVSVY
(SEQ ID NO: 114)
GAGACGTTCTCAGGTGGCTGCCTCTTTGATGAGCCGTATAGCACATGTGGATATA
GTCAATCTGAAGGTGATGACTTCAATTGGGAGCAAGTGAACACCTTGACTAAAC
CGACTTCTGATCCATGGATGCCATCAGGTTCTTTCATGCTGGTGAATGCCTCTGG
GAGACCTGAGGGGCAGAGAGCCCACCTGCTCTTACCCCAACTTAAAGAAAATGA
CACCCACTGCATCGATTTTCACTATTTTGTGTCCAGCAAGAGTAATTCTCCTCCGG
GGTTACTCAATGTCTACGTGAAGGTCAATAACGGGCCACTGGGGAATCCTATCTG
GAATATATCTGGAGACCCAACACGTACATGGAACAGGGCAGAACTGGCCATTAG
TACTTTCTGGCCTAACTTTTATCAGGTGATTTTTGAAGTGATAACTTCTGGACATC
AAGGCTATCTCGCTATCGATGAGGTGAAGGTGTTAGGACATCCATGTACCAGGA
CTCCTCACTTCCTGCGGATTCAGAATGTGGAAGTTAATGCTGGCCAGTTTGCTAC
CTTCCAGTGCAGTGCCATCGGCAGGACCGTGGCAGGAGACAGGCTCTGGTTACA
GGGCATTGATGTGCGAGATGCTCCTCTGAAGGAAATCAAGGTGACCAGCTCCCG
ACGCTTCATTGCTTCATTTAATGTTGTGAATACCACCAAACGAGATGCTGGAAAG
TACCGCTGCATGATTCGCACTGAAGGAGGTGTTGGAATATCAAACTATGCAGAG
TTGGTAGTTAAAGAACCACCCGTTCCTATTGCCCCACCTCAGCTCGCCTCTGTAG
GAGCCACCTACCTGTGGATACAGCTCAACGCCAACTCCATCAATGGGGATGGGC
CCATTGTGGCCCGAGAGGTGGAGTACTGCACGGCCAGTGGGAGCTGGAATGACC
GGCAGCCAGTCGATTCCACGAGCTATAAAATTGGACACCTTGACCCAGATACAG
AATATGAGATTAGTGTGCTCCTGACCAGGCCAGGGGAGGGTGGCACTGGCTCTC
CTGGTCCAGCTCTCAGGACAAGAACAAAGTGTGCTGATCCCATGCGAGGCCCAA
GAAAACTAGAAGTAGTGGAGGTCAAATCTCGGCAAATCACTATCCGCTGGGAGC
CATTTGGATATAATGTAACTCGTTGCCACAGTTATAATCTCACTGTCCACTACTGT
TACCAAGTTGGAGGACAAGAACAAGTGCGAGAAGAAGTAAGCTGGGATACAGA
AAATTCACACCCTCAACACACGATCACTAACCTGTCACCATACACCAATGTCAGT
GTGAAACTGATCCTCATGAACCCAGAGGGCCGGAAGGAAAGCCAAGAACTCATA
GTGCAGACAGATGAAGACCTCCCAGGTGCTGTTCCCACTGAATCCATACAAGGA
AGTACCTTTGAAGAGAAGATATTTCTTCAGTGGAGAGAACCAACTCAAACATAT
GGTGTAATCACTTTATATGAGATCACCTACAAAGCAGTCAGTTCCTTTGACCCAG
AAATAGATTTATCCAATCAGAGTGGAAGAGTTTCAAAGCTGGGAAATGAAACCC
ATTTTCTGTTTTTTGGACTGTATCCGGGGACCACATACTCCTTTACCATCCGAGCT
AGCACAGCTAAGGGTTTTGGGCCTCCAGCAACAAACCAGTTCACCACCAAAATA
TCAGCACCCTCTATGCCAGCTTATGAACTTGAGACACCTTTGAATCAAACTGACA
ATACCGTGACAGTCATGCTGAAACCTGCCCACAGCAGAGGAGCACCTGTCAGTG
TCTAT

| Name of scFv | AA Binding site | Subfamily Recognition |
|---|---|---|
| MAM-B | 42-60 | PTPmu |
| MAM-D | 42-60 | PTPmu |
| MAM-G | 42-60 | PTPmu |
| MAM-I | 42-60 | PTPmu |
| JM-F | 816-914 | PTPmu |
| JM-G | 816-914 | PTPmu, PTPkappa, PCP-2 |
| PTPD1-O | 958-1161 | PTPmu, PTPrho, PCP-2 |
| PTPD1-R | 915-958 | PTPmu and PTPrho |

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1: An antibody, antibody fragment or antigen binding fragment thereof comprising at least one of:
a)
a CDR-H1 comprising the amino acid sequence of GFTFTDYY (SEQ ID NO: 3),
a CDR-H2 comprising the amino acid sequence of IRNKANGYRT (SEQ ID NO: 4), and
a CDR-H3 comprising the amino acid sequence of ASLSAY (SEQ ID NO: 5);
b)
a CDR-L1 comprising the amino acid sequence of KSLLHSNGITY (SEQ ID NO: 8),
a CDR-L2 comprising the amino acid sequence of QMS (SEQ ID NO: 9), and
a CDR-L3 comprising the amino acid sequence of AQNLELWT (SEQ ID NO: 10);
c)
a CDR-H1 comprising the amino acid sequence of GASITSGH (SEQ ID NO: 13),
a CDR-H2 comprising the amino acid sequence of IRDSGTT (SEQ ID NO: 14), and
a CDR-H3 comprising the amino acid sequence of VTTPFVY (SEQ ID NO: 15);
d)
a CDR-L1 comprising the amino acid sequence of QDINSY (SEQ ID NO: 18),
a CDR-L2 comprising the amino acid sequence of RAN (SEQ ID NO: 19), and
a CDR-L3 comprising the amino acid sequence of LQYDEFPLT (SEQ ID NO: 20);
e)
a CDR-H1 comprising the amino acid sequence of GFSLFNYG (SEQ ID NO: 23),
a CDR-H2 comprising the amino acid sequence of IWADGTI (SEQ ID NO: 24), and
a CDR-H3 comprising the amino acid sequence of ARHFDY (SEQ ID NO: 25);
f)
a CDR-L1 comprising the amino acid sequence of QNIVHSNGNTY (SEQ ID NO: 28),
a CDR-L2 comprising the amino acid sequence of KVS (SEQ ID NO: 29), and
a CDR-L3 comprising FQGSRLPLT (SEQ ID NO: 30);
g)
a CDR-H1 comprising the amino acid sequence of GFIFSSYY (SEQ ID NO: 33),
a CDR-H2 comprising the amino acid sequence of ININGGST (SEQ ID NO: 34), and
a CDR-H3 comprising the amino acid sequence of GRHDHGYGWFAY (SEQ ID NO: 35);
h)
a CDR-L1 comprising the amino acid sequence of QNVGRN (SEQ ID NO: 38),
a CDR-L2 comprising the amino acid sequence of SAS (SEQ ID NO: 39), and
a CDR-L3 comprising the amino acid sequence of QQYNSYPWT (SEQ ID NO: 40);
i)
a CDR-H1 comprising the amino acid sequence of GYTFTTAG (SEQ ID NO: 43),
a CDR-H2 comprising the amino acid sequence of INTQSGVS (SEQ ID NO: 44), and
a CDR-H3 comprising the amino acid sequence of ARSNSGGFPY (SEQ ID NO: 45);
j)
a CDR-L1 comprising the amino acid sequence QSLFNSTTHKIY (SEQ ID NO: 48),
a CDR-L2 comprising the amino acid sequence FAS (SEQ ID NO: 49), and
a CDR-L3 comprising the amino acid sequence of QQHYTAPWT (SEQ ID NO: 50);
k)
a CDR-H1 comprising the amino acid sequence of GYTFTTVG (SEQ ID NO: 53),
a CDR-H2 comprising the amino acid sequence of INTHSGVS (SEQ ID NO: 54), and
a CDR-H3 comprising the amino acid sequence of ARGNSNYGFPY (SEQ ID NO: 55);
l)
a CDR-L1 comprising the amino acid sequence of EDIYNR (SEQ ID NO: 58),
a CDR-L2 comprising the amino acid sequence of GAT (SEQ ID NO: 59), and
a CDR-L3 comprising the amino acid sequence of QQYWSTPWT (SEQ ID NO: 60);
m)
a CDR-H1 comprising the amino acid sequence of GYSITSDYA (SEQ ID NO: 63),
a CDR-H2 comprising the amino acid sequence of ISYRSIT (SEQ ID NO: 64), and
a CDR-H3 comprising the amino acid sequence of ARFGYDGNY (SEQ ID NO: 65);
n)
a CDR-L1 comprising the amino acid sequence of QNIVHSNGDTY (SEQ ID NO: 68),
a CDR-L2 comprising the amino acid sequence of RVS (SEQ ID NO: 69), and
a CDR-L3 comprising the amino acid sequence of FQGSHVPLT (SEQ ID NO: 70);
o)
a CDR-H1 comprising the amino acid sequence of GYTFTDYN (SEQ ID NO: 73),
a CDR-H2 comprising the amino acid sequence of INPDNGAT (SEQ ID NO: 74), and
a CDR-H3 comprising the amino acid sequence of VRGFDY (SEQ ID NO: 75);
p)
a CDR-L1 comprising the amino acid sequence of QAIVHSNGNTY (SEQ ID NO: 78),
a CDR-L2 comprising the amino acid sequence of KVS (SEQ ID NO: 79), and
a CDR-L3 comprising the amino acid sequence of FQSSHIPWT (SEQ ID NO: 80); or
a heavy chain variable region and/or light chain variable region that competitively inhibits binding of a peptide, protein, isolated antibody, antibody fragment, or antigen binding fragment thereof comprising at least one of a), b), c), d), e), f), g), h), i), j), k), l), m), n), o), or p) to a CAM.

2: The antibody, antibody fragment, or antigen binding fragment thereof of claim 1, comprising: a heavy chain variable region that includes the 3 CDRs of one of SEQ ID NO: 1, SEQ ID NO: 11, SEQ ID NO:21, SEQ ID NO: 31, SEQ ID NO: 41, SEQ ID NO: 51, SEQ ID NO: 61, or SEQ ID NO: 71; or a heavy chain variable region that competitively inhibits binding of a peptide, protein, isolated antibody, antibody fragment or antigen binding fragment comprising at least one of a heavy chain variable region that includes the 3 CDRs of one of SEQ ID NO: 1, SEQ ID NO: 11, SEQ ID NO: 21, SEQ ID NO: 31, SEQ ID NO: 41, SEQ ID NO: 51, SEQ ID NO: 61, or SEQ ID NO: 71.

3: The antibody, antibody fragment, or antigen binding fragment thereof of claim 1, comprising a heavy chain variable region that includes an amino acid sequence at least about 70% identical to the amino acid sequence of one of SEQ ID NO: 1, SEQ ID NO: 11, SEQ ID NO: 21, SEQ ID NO: 31, SEQ ID NO: 41, SEQ ID NO: 51, SEQ ID NO: 61, or SEQ ID NO: 71.

4: The antibody, antibody fragment, or antigen binding fragment thereof of claim 1, comprising a light chain variable region that includes the 3 CDRs of one of SEQ ID NO: 6, SEQ ID NO: 16, SEQ ID NO:26, SEQ ID NO: 36, SEQ ID NO: 46, SEQ ID NO: 56, SEQ ID NO: 66, or SEQ ID NO: 76; or a light chain variable region that competitively inhibits binding of a peptide, protein, isolated antibody, antibody fragment or antigen binding fragment comprising at least one of a light chain variable region that includes the 3 CDRs of one of SEQ ID NO: 6, SEQ ID NO: 16, SEQ ID NO: 26, SEQ ID NO: 36, SEQ ID NO:46, SEQ ID NO: 56, SEQ ID NO: 66, or SEQ ID NO: 76.

5: The antibody, antibody fragment, or antigen binding fragment thereof of claim 1, comprising a light chain variable region that includes an amino acid sequence at least about 70% identical to the amino acid sequence of one of SEQ ID NO: 6, SEQ ID NO: 16, SEQ ID NO: 26, SEQ ID NO: 36, SEQ ID NO: 46, SEQ ID NO: 56, SEQ ID NO: 66, or SEQ ID NO: 76.

6: The antibody, antibody fragment, or antigen binding fragment thereof of claim 1, comprising:
at least one of the following:
a) a heavy chain variable region that includes the 3 CDRs of SEQ ID NO: 1 and a light chain variable region that includes the 3 CDRs of SEQ ID NO: 6;
b) a heavy chain variable region that includes the 3 CDRs of SEQ ID NO: 11 and a light chain variable region that includes the 3 CDRs of SEQ ID NO: 16;
c) a heavy chain variable region that includes the 3 CDRs of SEQ ID NO: 21 and a light chain variable region that includes the 3 CDRs of SEQ ID NO: 26;
d) a heavy chain variable region that includes the 3 CDRs of SEQ ID NO: 31 and a light chain variable region that includes the 3 CDRs of SEQ ID NO: 36;
e) a heavy chain variable region that includes the 3 CDRs of SEQ ID NO: 41 and a light chain variable region that includes the 3 CDRs of SEQ ID NO: 46;
f) a heavy chain variable region that includes the 3 CDRs of SEQ ID NO: 51 and a light chain variable region that includes the 3 CDRs of SEQ ID NO: 56;
g) a heavy chain variable region that includes the 3 CDRs of SEQ ID NO: 61 and a light chain variable region that includes the 3 CDRs of SEQ ID NO: 66; or
h) a heavy chain variable region that includes the 3 CDRs of SEQ ID NO: 71 and a light chain variable region that includes the 3 CDRs of SEQ ID NO: 76.

7: The antibody, antibody fragment, or antigen binding fragment thereof of claim 1, comprising:
at least one of the following:
i) a heavy chain that includes an amino acid sequence at least about 70% identical to the amino acid sequence of SEQ ID NO: 1 and a light chain that includes an amino acid sequence at least about 70% identical to the amino acid sequence of SEQ ID NO: 6;
j) a heavy chain that includes an amino acid sequence at least about 70% identical to the amino acid sequence of SEQ ID NO: 11 and a light chain that includes an amino acid sequence at least about 70% identical to the amino acid sequence of SEQ ID NO: 16;
k) a heavy chain that includes an amino acid sequence at least about 70% identical to the amino acid sequence of SEQ ID NO: 21 and a light chain that includes an amino acid sequence at least about 70% identical to the amino acid sequence of SEQ ID NO: 26;
l) a heavy chain that includes an amino acid sequence at least about 70% identical to the amino acid sequence of SEQ ID NO: 31 and a light chain that includes an amino acid sequence at least about 70% identical to the amino acid sequence of SEQ ID NO: 36;
m) a heavy chain that includes an amino acid sequence at least about 70% identical to the amino acid sequency SEQ ID NO: 41 and a light chain that includes an amino acid sequence of at least 70% identical to the amino acid sequence of SEQ ID NO: 45;
n) a heavy chain that includes an amino acid sequence at least about 70% identical to the amino acid sequence of SEQ ID NO: 51
and a light chain that includes an amino acid sequence at least about 70% identical to the amino acid sequence of SEQ ID NO: 56;
o) a heavy chain that includes an amino acid sequence at least about 70% identical to the amino acid sequence of SEQ ID NO: 61 and a light chain that includes an amino acid sequence at least about 70% identical to the amino acid sequence of SEQ ID NO: 66; or
p) a heavy chain that includes an amino acid sequence at least about 70% identical to the amino acid sequence of SEQ ID NO: 71 and a light chain that includes an amino acid sequence at least about 70% identical to the amino acid sequence of SEQ ID NO: 76.

8: The antibody, antibody fragment, or antigen binding fragment thereof of claim 1 further comprising a peptide linker.

9: The antibody, antibody fragment, or antigen binding fragment thereof of claim 1, is humanized or chimeric.

10: The antibody, antibody fragment, or antigen binding fragment thereof of claim 1, binds to PTPμ, PTPκ, PCP-2, PTPμ, or PTPρ.

11. (canceled)

12: The antibody, antibody fragment, or antigen binding fragment of claim 1, including an amino acid sequence at least about 70% identical to SEQ ID NO: 81, SEQ ID NO: 83, SEQ ID NO: 85, SEQ ID NO: 87, SEQ ID NO: 89, SEQ ID NO: 91, SEQ ID NO: 93, SEQ ID NO: 95, SEQ ID NO: 97, SEQ ID NO: 99, SEQ ID NO: 101, SEQ ID NO: 103, SEQ ID NO: 105, SEQ ID NO: 107, SEQ ID NO: 109, or SEQ ID NO: 111.

13: The antibody, antibody fragment, or antigen binding fragment of claim 1 including an amino acid sequence encoded by a nucleotide sequence at least about 70% identical to SEQ ID NO: 82, SEQ ID NO: 84, SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 102, SEQ ID NO: 104, SEQ ID NO: 106, SEQ ID NO: 108, SEQ ID NO: 110, or SEQ ID NO: 112.

14: A chimeric antigen receptor (CAR) comprising extracellular antigen binding domain that includes an scFv having at least one of:
a)
a CDR-H1 comprising the amino acid sequence of GFTFTDYY (SEQ ID NO: 3),
a CDR-H2 comprising the amino acid sequence of IRNKANGYRT (SEQ ID NO: 4), and
a CDR-H3 comprising the amino acid sequence of ASLSAY (SEQ ID NO: 5);
b)
a CDR-L1 comprising the amino acid sequence of KSLLHSNGITY (SEQ ID NO: 8),
a CDR-L2 comprising the amino acid sequence of QMS (SEQ ID NO: 9), and
a CDR-L3 comprising the amino acid sequence of AQNLELWT (SEQ ID NO: 10);
c)
a CDR-H1 comprising the amino acid sequence of GASITSGH (SEQ ID NO: 13),
a CDR-H2 comprising the amino acid sequence of IRDSGTT (SEQ ID NO: 14), and
a CDR-H3 comprising the amino acid sequence of VTTPFVY (SEQ ID NO: 15);
d)
a CDR-L1 comprising the amino acid sequence of QDINSY (SEQ ID NO: 18),
a CDR-L2 comprising the amino acid sequence of RAN (SEQ ID NO: 19), and
a CDR-L3 comprising the amino acid sequence of LQYDEFPLT (SEQ ID NO: 20);
e)
a CDR-H1 comprising the amino acid sequence of GFSLFNYG (SEQ ID NO: 23),
a CDR-H2 comprising the amino acid sequence of IWADGTI (SEQ ID NO: 24), and
a CDR-H3 comprising the amino acid sequence of ARHFDY (SEQ ID NO: 25);
f)
a CDR-L1 comprising the amino acid sequence of QNIVHSNGNTY (SEQ ID NO: 28),
a CDR-L2 comprising the amino acid sequence of KVS (SEQ ID NO: 29), and
a CDR-L3 comprising FQGSRLPLT (SEQ ID NO: 30);
g)
a CDR-H1 comprising the amino acid sequence of GFIFSSYY (SEQ ID NO: 33),
a CDR-H2 comprising the amino acid sequence of ININGGST (SEQ ID NO: 34), and
a CDR-H3 comprising the amino acid sequence of GRHDHGYGWFAY (SEQ ID NO: 35);
h)
a CDR-L1 comprising the amino acid sequence of QNVGRN (SEQ ID NO: 38),
a CDR-L2 comprising the amino acid sequence of SAS (SEQ ID NO: 39), and
a CDR-L3 comprising the amino acid sequence of QQYNSYPWT (SEQ ID NO: 40);
i)
a CDR-H1 comprising the amino acid sequence of GYTFTTAG (SEQ ID NO: 43),
a CDR-H2 comprising the amino acid sequence of INTQSGVS (SEQ ID NO: 44), and
a CDR-H3 comprising the amino acid sequence of ARSNSGGFPY (SEQ ID NO: 45);
j)
a CDR-L1 comprising the amino acid sequence QSLFNSTTHKIY (SEQ ID NO: 48),
a CDR-L2 comprising the amino acid sequence FAS (SEQ ID NO: 49), and
a CDR-L3 comprising the amino acid sequence of QQHYTAPWT (SEQ ID NO: 50);
k)
a CDR-H1 comprising the amino acid sequence of GYTFTTVG (SEQ ID NO: 53),
a CDR-H2 comprising the amino acid sequence of INTHSGVS (SEQ ID NO: 54), and
a CDR-H3 comprising the amino acid sequence of ARGNSNYGFPY (SEQ ID NO: 55);
l)
a CDR-L1 comprising the amino acid sequence of EDIYNR (SEQ ID NO: 58),
a CDR-L2 comprising the amino acid sequence of GAT (SEQ ID NO: 59), and
a CDR-L3 comprising the amino acid sequence of QQYWSTPWT (SEQ ID NO: 60);
m)
a CDR-H1 comprising the amino acid sequence of GYSITSDYA (SEQ ID NO: 63),
a CDR-H2 comprising the amino acid sequence of ISYRSIT (SEQ ID NO: 64), and
a CDR-H3 comprising the amino acid sequence of ARFGYDGNY (SEQ ID NO: 65);
n)
a CDR-L1 comprising the amino acid sequence of QNIVHSNGDTY (SEQ ID NO: 68),
a CDR-L2 comprising the amino acid sequence of RVS (SEQ ID NO: 69), and
a CDR-L3 comprising the amino acid sequence of FQGSHVPLT (SEQ ID NO: 70);
o)
a CDR-H1 comprising the amino acid sequence of GYTFTDYN (SEQ ID NO: 73),
a CDR-H2 comprising the amino acid sequence of INPDNGAT (SEQ ID NO: 74), and
a CDR-H3 comprising the amino acid sequence of VRGFDY (SEQ ID NO: 75);
q)
a CDR-L1 comprising the amino acid sequence of QAIVHSNGNTY (SEQ ID NO: 78),
a CDR-L2 comprising the amino acid sequence of KVS (SEQ ID NO: 79), and
a CDR-L3 comprising the amino acid sequence of FQSSHIPWT (SEQ ID NO: 80); or
a heavy chain variable region and/or light chain variable region that competitively inhibits binding of a peptide, protein, isolated antibody, antibody fragment, or antigen binding fragment thereof comprising at least one of a), b), c), d), e), f), g), h), i), j), k), l), m), n), o), p), or q) to a CAM.

15: The CAR of claim 14, wherein the extracellular antigen binding domain binds to a PTPμ ligand having an amino acid sequence at least about 70% identical to SEQ ID NO: 113.

16: A nucleic acid comprising a nucleotide sequence encoding the CAR of claim 14.

17: The nucleic acid of claim 16, wherein the nucleotide is operably linked to a promoter.

18: An expression construct comprising the nucleic acid of claim 16.

19: The expression construct of claim 18, comprising a vector.

20: The expression construct of claim 19, wherein the vector is a retroviral vector, a lentiviral vector, or an AAV vector.

21: A therapeutic agent or diagnostic agent comprising the antibody, antibody fragment, or antigen binding fragment thereof of claim 1.

22: A therapeutic agent or diagnostic agent comprising the CAR of claim 14.
